(19) United States
(12) Patent Application Publication
(10) Pub. No.: US 2021/0285000 A1
(43) Pub. Date: Sep. 16, 2021

(54) COMBINATION THERAPY FOR TREATING HEPATITIS B VIRUS INFECTION

(71) Applicant: Janssen Pharmaceuticals, Inc., Titusville, NJ (US)

(72) Inventors: Ronald Cornelis Marie KALMEIJER, Titusville, NJ (US); Michael BIERMER, Berlin (DE); Oliver LENZ, Sint-Katelijne-Waver (BE); Heather Lynn DAVIS, Schilde (BE); Cristiana MAYER, Pennington, NJ (US)

(21) Appl. No.: 17/191,870

(22) Filed: Mar. 4, 2021

Related U.S. Application Data
(60) Provisional application No. 62/985,588, filed on Mar. 5, 2020.
(60) Provisional application No. 63/092,692, filed on Oct. 16, 2020.

(51) Int. Cl. C12N 15/113; A61K 31/713; A61K 31/40; A61K 31/4025; A61K 38/21; A61P 31/20

(52) U.S. Cl. CPC C12N 15/113; A61K 31/713; A61K 31/40; A61K 31/4025; C12N 2310/351; A61P 31/20; C12N 2310/14; C12N 2320/31; A61K 38/212

(57) ABSTRACT

Described are RNA interference (RNAi) agents for inhibiting the expression of Hepatitis B Virus (HBV) used in combination with a capsid assembly modulator (CAM) and an interferon, and methods of administering same. The HBV RNAi agents, CAMs and interferon are administered to effectively inhibit HBV gene expression and to treat diseases and conditions associated with HBV infection, particularly in immune tolerant subjects.

### CROSS REFERENCE TO RELATED APPLICATION

This application claims priority to U.S. Provisional Application No. 63/092,692 filed on Oct. 16, 2020 and U.S. Provisional Application No. 62/985,588 filed on Mar. 5, 2020, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “Sequence_Listing_065814_11286” and a creation date of Feb. 15, 2021 and having a size of 4.15 kb. The sequence listing submitted via EFS-Web is part of the specification and is herein incorporated by reference in its entirety.

### FIELD OF INVENTION

The present disclosure relates generally to compositions and kits comprising an RNA interference (RNAi) component, a capsid assembly modulator and an interferon, their uses for treating hepatitis B virus infection or inhibiting the expression of at least one hepatitis B virus gene.

### BACKGROUND

The hepatitis B virus (HBV) is a strict hepatotrophic, double-stranded DNA containing virus. Although DNA is the genetic material, the replication cycle involves a reverse transcription step to copy a pregenomic RNA into DNA. Hepatitis B virus is classified as one member of the Hepadnaviruses and belongs to the family of Hepadnaviridae. The primary infection of adult humans with hepatitis B virus causes an acute hepatitis with symptoms of organ inflammation, fever, jaundice and increased liver transaminases in blood. Those patients that are not able to overcome the virus infection suffer a chronic disease progression over many years with increased risk of developing cirrhotic liver or liver cancer. Perinatal transmission from hepatitis B virus-infected mothers to newborns also leads to chronic hepatitis.

Upon uptake by hepatocytes, the nucleocapsid is transferred to the nucleus and DNA is released. There, the DNA strand synthesis is completed and gaps repaired to give the covalently closed circular (ccc) supercoiled DNA of 3.2 kb. The cccDNA serves as a template for transcription of five major viral mRNAs, which are 3.5, 3.5, 2.4, 2.1 and 0.7 kb long. All mRNAs are 5′-capped and polyadenylated at the 3′-end. There is sequence overlap at the 3′-end between all five mRNAs.

One 3.5 kb mRNA serves as template for core protein and polymerase production. In addition, the same transcript serves as a pre-genomic replication intermediate and allows the viral polymerase to initiate the reverse transcription into DNA. Core protein is needed for nucleocapsid formation. The other 3.5 kb mRNA encodes pre-core, the secretable e-antigen (HBeAg). In the absence of replication inhibitors, the abundance of e-antigen in blood correlates with Hepatitis B Virus replication in liver and serves as an important diagnostic marker for monitoring the disease progression.

The 2.4 and 2.1 kb mRNAs carry the open reading frames (“ORF”) pre-S1, pre-S2 and S for expression of viral large, medium and small surface antigen. The s-antigen is associated with infectious, complete particles. In addition, blood of infected patients also contains non-infectious particles derived from s-antigen alone, free of genomic DNA or polymerase. The function of these particles is not fully understood. The complete and lasting depletion of detectable s-antigen in blood is considered as a reliable indicator for hepatitis B virus clearance.

The 0.7 kb mRNA encodes the X protein. This gene product is important for efficient transcription of viral genes and also acts as a transactivator on host gene expression. The latter activity seems to be important for hepatocyte transformation during development of liver cancer.

Patients with detectable s-antigen (HBsAg), e-antigen (HBeAg), and/or viral DNA in the blood for more than 6 months are considered chronically infected. Chronic HBV infection can be classified into five phases: (I) HBeAg-positive chronic infection, (II) HBeAg-positive chronic hepatitis, (III) HBeAg-negative chronic infection, (IV) HBeAg-negative chronic hepatitis and (V) HBsAg-negative phase. All patients with chronic HBV infection are at increased risk of progression to cirrhosis and hepatocellular carcinoma (HCC), depending on host and viral factors (Lampertico et al., J Hepatol., 2017, 67(2):370-398). At the present, a complete sterilizing cure, i.e., viral eradication from the host, is unlikely to be feasible (Lok et al., J Hepatol., 2017, 67(4):847-861). The main goal of therapy is to improve survival and quality of life by preventing disease progression, and consequently HCC development. The induction of long-term suppression of HBV replication represents the main endpoint of current treatment strategies, while HBsAg loss is an optimal endpoint.

Nucleoside analogs as inhibitors of reverse transcriptase activity are typically the first treatment option for many patients. Long term administration of lamivudine, tenofovir, and/or entecavir has been shown to suppress hepatitis B virus replication, sometimes to undetectable levels, with improvement of liver function and reduction of liver inflammation typically seen as the most important benefits. However, only few patients achieve complete and lasting remission after the end of treatment. Furthermore, the hepatitis B virus develops drug resistance with increasing duration of treatment. This is especially difficult for patients co-infected with hepatitis B and human immunodeficiency virus (HIV). Both viruses are susceptible to nucleoside analogue drugs and may co-develop resistance.

Pegylated interferon-alpha (IFN) has been used to treat mild to moderate chronic hepatitis B patients. However, current treatment of chronic hepatitis B has limited efficacy (Erha et al., Gut. 2005 July; 54(7): 1009-1013). For example, the Asian genotype B gives very poor response rates. Co-infection with hepatitis D virus (HDV) or human immunodeficiency virus has been shown to render interferon-alpha therapy completely ineffective. Patients with strong liver damage and heavy fibrotic conditions are not qualified for interferon-alpha therapy.

Certain hepatitis B virus-specific RNA interference (RNAi) agents have been previously shown to inhibit expression of HBV gene expression. For example, U.S. Patent Application Publication No. 2013/0005793, to Chin et al., which is incorporated herein by reference in its entirety, discloses certain double-stranded ribonucleic acid (dsRNA) molecules for inhibiting the expression of hepatitis B virus gene.

Additionally, HBV inhibitors, such as capsid assembly modulator (CAM), can bind to hepatitis B core protein and interferes with the viral capsid assembly process, thereby preventing the polymerase-bound pgRNA encapsidation. This results in the formation of HBV capsids, devoid of HBV DNA or RNA (non-functional capsids), and ultimately in the inhibition of HBV replication. See e.g., WO2014184350A1. The reference WO2014184350A1 is incorporated herein in its entirety, particularly the descriptions of the capsid assembly modulator compounds and the method of preparing them.

Several combinations have been tried in the treatment of HBV in various settings (Paul, Curr Hepat Rep. 2011 June; 10(2): 98-105). However, various combination therapies for HBeAg-positive chronic HBV or HBeAg-negative hepatitis B have not established a benefit over monotherapy. Id.

There is a need for improved HBV therapy that can overcome at least one of the disadvantages of existing treatment options, such as toxicity, mutagenicity, lack of selectivity, poor efficacy, poor bioavailability, and difficulty of synthesis, while providing additional benefits such as increased potency or an increased safety window.

The disclosures of all publications, patents, patent applications and published patent applications referred to herein are hereby incorporated herein by reference in their entirety.

### BRIEF SUMMARY

Provided herein is a method of inhibiting the expression of a hepatitis B virus gene, treating a hepatitis B virus infection or a disease or disorder associated with an infection caused by hepatitis B virus in a subject in need thereof, wherein the method comprises administering to the subject:

(1) an effective amount of a pharmaceutical composition comprising an RNAi component having:
(i) a first RNAi agent comprising: an antisense strand comprising a nucleotide sequence of any one of the following: SEQ ID NOT, SEQ ID NO:2, SEQ ID NOT, SEQ ID NO:4, SEQ ID NOT, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NOT and SEQ ID NO:9, and a sense strand comprising a nucleotide sequence of any one of the following: SEQ ID NO: 16, SEQ ID NO: 17, SEQ ID NO: 18, and SEQ ID NO: 19;

(2) an effective amount of a pharmaceutical composition comprising an effective amount of a compound of Formula (I):

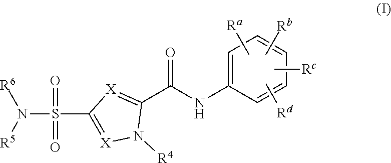

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and

(3) an effective amount of a pharmaceutical composition comprising an effective amount of an interferon, such as an interferon alpha or lambda, preferably a pegylated interferon, more preferably a pegylated interferon alpha-2a or pegylated interferon lambda-1a.

The application also relates to a method of treating a hepatitis B virus infection or a disease or disorder associated with an infection caused by hepatitis B virus in a subject receiving a capsid assembly modulator therapy, wherein the method further comprises administering to the subject:
(1) a pharmaceutical composition comprising an effective amount of an RNAi component, wherein the RNAi component comprises
   (i) a first RNAi agent comprising: an antisense strand comprising a nucleotide sequence of any one of the following: SEQ ID NOT, SEQ ID NO:2, SEQ ID NO:3, SEQ ID NO:4, SEQ ID NO:5, SEQ ID NO:6, and SEQ ID NOT and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
   (ii) a second RNAi agent comprising: an antisense strand comprising a nucleotide sequence of any one of the following: SEQ ID NO: 8 and SEQ ID NO:9, and a sense strand comprising a nucleotide sequence of any one of the following: SEQ ID NO: 16, SEQ ID NO: 17, SEQ ID NO: 18, and SEQ ID NO: 19, and
(2) a pharmaceutical composition comprising an effective amount of a third pharmaceutical composition comprising an effective amount of an interferon, such as an interferon alpha or lambda, preferably a pegylated interferon, more preferably a pegylated interferon alpha-2a or pegylated interferon lambda-1a.

The application also relates to a method of treating a hepatitis B virus infection or a disease or disorder associated with an infection caused by hepatitis B virus in a subject receiving an RNAi therapy, wherein the method further comprises administering to the subject:

(1) an effective amount of a pharmaceutical composition comprising an effective amount of a compound of Formula (I):

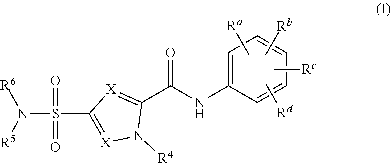

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰; R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and

(3) an effective amount of a pharmaceutical composition comprising an effective amount of an interferon, such as an interferon alpha or lambda, preferably a pegylated interferon, more preferably a pegylated interferon alpha-2a or pegylated interferon lambda-1a.

The application also relates to a method of treating a hepatitis B virus infection or a disease or disorder associated with an infection caused by hepatitis B virus in a subject receiving an interferon therapy, wherein the method further comprises administering to the subject:

(1) an effective amount of a pharmaceutical composition comprising an RNAi component having:
(i) a first RNAi agent comprising: an antisense strand comprising a nucleotide sequence of any one of the following: SEQ ID NOT, SEQ ID NO:2, SEQ ID NOT, SEQ ID NO:4, SEQ ID NOT, SEQ ID NO:6, and SEQ ID NOT and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NOT and SEQ ID NO:9, and a sense strand comprising a nucleotide sequence of any one of the following: SEQ ID NO: 16, SEQ ID NO: 17, SEQ ID NO: 18, and SEQ ID NO: 19; and

(2) an effective amount of a pharmaceutical composition comprising an effective amount of a compound of Formula (I):

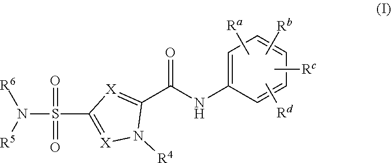

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments, the method further comprises administering to the subject another agent for treating infection caused by hepatitis B virus HBV. The other agent can be a nucleoside analog. In some embodiments, the nucleoside analog is entecavir, tenofovir disoproxil fumarate or tenofovir alafenamide.

In some embodiment, the subject has chronic HBV infection. Preferably, the subject is immune tolerant.

Another general aspect of the application relates to a combination or a kit comprising:

(1) a pharmaceutical composition comprising an RNAi component having:
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NO: 8 and SEQ ID NO:9, and a sense strand comprising a nucleotide sequence of any one of the following: SEQ ID NO: 16, SEQ ID NO: 17, SEQ ID NO: 18, and SEQ ID NO: 19;

(2) a pharmaceutical composition comprising an effective amount of a compound of Formula (I):

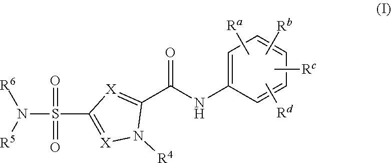

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and

(3) a pharmaceutical composition comprising an effective amount of an interferon, such as an interferon alpha or lambda, preferably a pegylated interferon, more preferably a pegylated interferon alpha-2a or pegylated interferon lambda-1a. The pharmaceutical compositions of (1), (2) and (3) advantageously are for simultaneous, sequential or separate use.

In some embodiments, the combination or kit further comprises another agent for treating infection caused by hepatitis B virus HBV. The other agent can be a nucleoside analog. In some embodiments, the nucleoside analog is entecavir, tenofovir disoproxil fumarate or tenofovir alafenamide.

Also provided herein is a pharmaceutical composition for use in a treatment of HBV infection or a disease or disorder associated with an HBV infection in a subject in need thereof, wherein the pharmaceutical composition comprises a RNAi component comprising:
(i) a first RNAi agent comprising: an antisense strand comprising a nucleotide sequence of any one of the following: SEQ ID NOT, SEQ ID NO:2, SEQ ID NOT, SEQ ID NOT, SEQ ID NOT, SEQ ID NO:6, and SEQ ID NOT and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NOT and SEQ ID NOV, and a sense strand comprising a nucleotide sequence of any one of the following: SEQ ID NO: 16, SEQ ID NO: 17, SEQ ID NO: 18, and SEQ ID NO: 19; and
   wherein the treatment comprises:
(a) the administration of the pharmaceutical composition,
(b) the administration of a compound of formula (I):

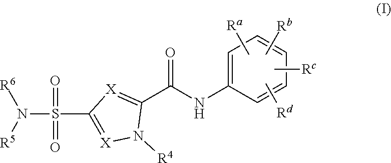

or a pharmaceutically acceptable salt thereof, wherein:
   each X is independently CR⁷;
   R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
   R^{d} is hydrogen or fluoro;
   R⁴ is hydrogen, C₁-C₃ alkyl or C₃-C₄ cycloalkyl;
   R⁵ is hydrogen;
   R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
   R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
   R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
   R⁹ is C₁-C₄ alkyloxy, —SO₂-methyl, —C(═O)—OR¹¹ or —C(═O)—N(R¹¹)₂;
   R¹⁰ is —CN, —OH, fluoro, —CHF₂, —CH₂F or —CF₃; and
   R¹¹ is hydrogen or C₁-C₃ alkyl; and
(c) the administration of interferon, such as an interferon alpha or lambda, preferably a pegylated interferon, more preferably a pegylated interferon alpha-2a or pegylated interferon lambda-1a.

Also provided herein is a pharmaceutical composition comprising a compound of Formula (I) or a pharmaceutically acceptable salt thereof for use in a treatment of an HBV infection or a disease or disorder associated with the HBV infection in a subject in need thereof, wherein the compound of Formula (I) is:

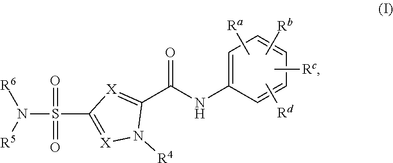

wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and
   wherein the treatment comprises:
(a) the administration of the pharmaceutical composition,
(b) the administration of a RNAi component, which comprises:
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO: 16, SEQ ID NO: 17, SEQ ID NO: 18, and SEQ ID NO: 19; and
(c) the administration of an interferon, such as an interferon alpha or lambda, preferably a pegylated interferon, more preferably a pegylated interferon alpha-2a or pegylated interferon lambda-1a.

Also provided herein is a pharmaceutical composition comprising an interferon for use in a treatment of an HBV infection or a disease or disorder associated with the HBV infection in a subject in need thereof, wherein the interferon is, for example, an interferon alpha or lambda, preferably a pegylated interferon, more preferably a pegylated interferon alpha-2a or pegylated interferon lambda-1a and the treatment comprises:
(a) the administration of the pharmaceutical composition,
(b) the administration of a RNAi component, which comprises:
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO: 16, SEQ ID NO: 17, SEQ ID NO: 18, and SEQ ID NO: 19; and
(c) the administration of a compound of Formula (I):

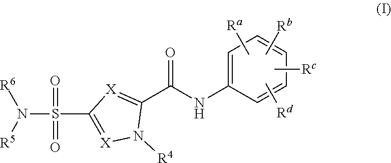

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments of the pharmaceutical composition for use described above, the treatment further comprises the administration of a nucleotide analog or a nucleoside analog, the nucleotide analog or nucleoside analog is preferably selected from entecavir, tenofovir disoproxil fumarate, tenofovir disoproxil maleate, or tenofovir alafenamide.

Also provided herein is an RNAi component, a compound of Formula (I) or a pharmaceutically acceptable salt thereof and an interferon, optionally a nucleoside analog, in the manufacture of a medicament or a combination of medicaments for treating an HBV infection or a disease or disorder associated with the HBV infection in a subject in need thereof, wherein:
(a) the RNAi component comprises:
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
   (ii) a second RNAi agent comprising: an antisense strand comprising a nucleotide sequence of any one of the following: SEQ ID NO: 8 and SEQ ID NOV, and a sense strand comprising a nucleotide sequence of any one of the following: SEQ ID NO: 16, SEQ ID NO: 17, SEQ ID NO: 18, and SEQ ID NO: 19; and
(b) the compound of Formula (I) is

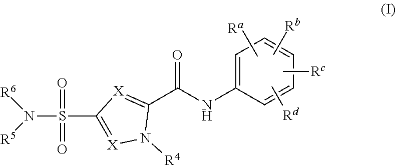

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl, and
(c) the interferon is preferably an interferon alpha or lambda, preferably a pegylated interferon, more preferably a pegylated interferon alpha-2a or pegylated interferon lambda-1a; and
(d) the nucleoside analog is preferably entecavir, tenofovir disoproxil fumarate or tenofovir alafenamide.

In preferred embodiments, the compound of Formula (I) is a compound of

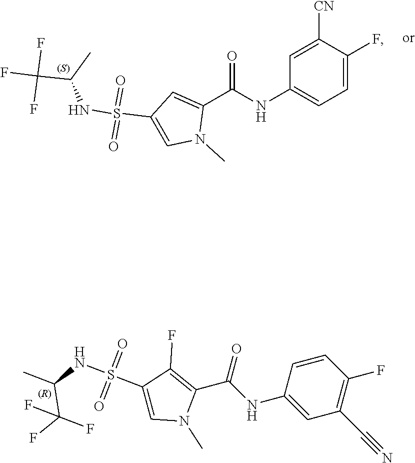

or a pharmaceutically acceptable salt thereof.

In any of the methods or other disclosures herein, in one variation the first or the second RNAi agent comprises at least one modified nucleotide or at least one modified internucleoside linkage. In another variation, substantially all of the nucleotides in the first and the second RNAi agents are modified nucleotides. In a further variation, the first or the second RNAi agent further comprises a targeting ligand that is conjugated to the first or the second RNAi agent. In one aspect, the targeting ligand comprises N-acetyl-galactosamine. In a particular aspect, the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s. In one variation, the targeting ligand is (NAG25), (NAG25)s, (NAG31), (NAG31)s, (NAG37), or (NAG37)s. In another variation, the targeting ligand is conjugated to the sense strand of the first or the second RNAi agent. In another variation, the targeting ligand is conjugated to the 5′ terminus of the sense stand of the first or the second RNAi agent. In still another variation, the first and the second RNAi agents independently comprise a duplex selected from the group consisting of: an antisense strand comprising SEQ ID NO: 1 and a sense strand comprising SEQ ID NO: 10; an antisense strand comprising SEQ ID NO:2 and a sense strand comprising SEQ ID NO: 11; an antisense strand comprising SEQ ID NO: 3 and a sense strand comprising SEQ ID NO: 11; an antisense strand comprising SEQ ID NO: 4 and a sense strand comprising SEQ ID NO: 12; an antisense strand comprising SEQ ID NO: 8 and a sense strand comprising SEQ ID NO: 16; an antisense strand comprising SEQ ID NO: 8 and a sense strand comprising SEQ ID NO: 17; an antisense strand comprising SEQ ID NO:2 and a sense strand comprising SEQ ID NO: 13; and an antisense strand comprising SEQ ID NO: 8 and a sense strand comprising SEQ ID NO: 18. In a particular variation, the first and the second RNAi agents are each independently conjugated to a targeting ligand comprising N-acetyl-galactosamine, and the first and the second RNAi agents independently comprise a duplex selected from the group consisting of: an antisense strand comprising SEQ ID NO:2 and a sense strand comprising SEQ ID NO: 11; an antisense strand comprising SEQ ID NO: 4 and a sense strand comprising SEQ ID NO: 12; an antisense strand comprising SEQ ID NO: 8 and a sense strand comprising SEQ ID NO: 16; an antisense strand comprising SEQ ID NO:2 and a sense strand comprising SEQ ID NO: 13; and an antisense strand comprising SEQ ID NO: 8 and a sense strand comprising SEQ ID NO: 18. In still another variation, the ratio of the first RNAi agent to the second RNAi agent by weight is in the range of about 1:2 to about 5:1. In another variation, the ratio of the first RNAi agent to the second RNAi agent by weight is about 2:1. In certain aspects, the first and the second RNAi agents are each independently conjugated to (NAG37)s, the first RNAi agent comprises an antisense strand comprising SEQ ID NO: 2 and a sense strand comprising SEQ ID NO: 11, and the second RNAi agent comprises an antisense strand comprising SEQ ID NO: 8 and a sense strand comprising SEQ ID NO: 16.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 shows a schematic overview of the study of example 1.

FIG. 2 shows a schematic overview of the study of example 2.

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged and caged ring groups (e.g., norbomane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via anon-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

As used herein, the term “3-7 membered mono or polycyclic saturated ring” means saturated cyclic hydrocarbon with 3, 4, 5, 6 or 7 carbon atoms and is generic to cyclopropyl, cyclobutyl, cyclopentyl, cyclohexyl and cycloheptyl (monocyclic) and fused or spiro ring systems with 2 or more saturated rings with at most 7 carbon atoms (polycyclic). Such saturated ring optionally contains one or more heteroatoms, such that at least one carbon atom is replaced by a heteroatom selected from N, O and S, in particular from N and O. Examples include oxetane, tetrahydro-2H-pyranyl, piperidinyl, tetrahydro-furanyl, morpholinyl, thiolane 1,1-dioxide and pyrrolidinyl. Preferred are saturated cyclic hydrocarbon with 3 or 4 carbon atoms and 1 oxygen atom. Examples include oxetane, and tetrahydrofuranyl.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl, oxo, and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethyl amine, diethylamine, tri ethyl amine, tripropylamine, and ethanolamine. In some embodiments, the pharmaceutically acceptable base addition salt is selected from ammonium, potassium, sodium, calcium, and magnesium salts.

The terms “patient” and “subject” refer to an animal, such as a mammal, bird, or fish. In some embodiments, the patient or subject is a mammal. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human. In some embodiments, the patient or subject is a human, for example a human that has been or will be the object of treatment, observation or experiment. The compounds, compositions and methods described herein can be useful in both human therapy and veterinary applications.

Chronic HBV infection can be classified into four phases, which typically, but not always, progress from one to the next: (I) HBeAg-positive chronic infection (previously known as immune tolerant), (II) HBeAg-positive chronic hepatitis (previously known as immune active), (III) HBeAg-negative chronic infection (previously known as inactive carrier), and (IV) HBeAg-negative chronic hepatitis (previously known as reactivation. The different phases of chronic HBV infection can also be characterized by differences in viral load, liver enzyme levels (necroinflammatory activity), HBeAg, or HBsAg load or presence of antibodies to these antigens. cccDNA levels in untreated subjects stay relatively constant at approximately 10 to 50 copies per cell, but can be as low as 1 to 2 copies per cell when suppressed by nucleos(t)ide analogue therapy, even though viremia can vary considerably. The persistence of the cccDNA species leads to chronicity. In some embodiment, a chronic HBV infection can be characterized by the laboratory criteria published by the Centers for Disease Control and Prevention (CDC), such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

As used herein, an “immune tolerant subject” or “immune tolerant patient” refers to a subject who is HBeAg positive with the HBV viral DNA levels at or above 10,000 IU/mL and has a normal alanine aminotransferase (ALT) level. In some embodiments, an “immune tolerant subject” or “immune tolerant patient” has a serum HBV DNA level at or above 10,000 IU/mL, more particularly at or above 20,000 IU/mL, more particularly at or above 10,000,000 IU/mL, and the subject has a normal serum ALT level, more particularly for male subjects or patients: a serum ALT level at or below 43 U/L, more particularly at or below 40 U/L, more particularly at or below 30 U/L, for female subjects or patients: a serum ALT level at or below 43 U/L, more particularly at or below 36 U/L, more particularly at or below 30 U/L. An “immune tolerant subject” or “immune tolerant patient” usually is HBeAg-positive.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “therapeutically effective amount” or “effective amount” refers to that amount of a compound disclosed and/or described herein that is sufficient to affect treatment, as defined herein, when administered to a subject in need of such treatment. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount can be ascertained experimentally, for example by assaying blood concentration of the compound, or theoretically, by calculating bioavailability by one of ordinary skill in the art in view of the present disclosure.

In particular embodiments of the application, a therapeutically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

A therapeutically effective amount can also be an amount of the compound sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

As used herein, the terms and phrases “in combination,” “in combination with,” “co-delivery,” and “administered together with” in the context of the administration of two or more therapies or components to a subject refers to simultaneous administration, overlapping administration or subsequent administration of two or more therapies or components. “Simultaneous administration” or “simultaneously administered” refers to administration of the two or more therapies or components within the same treatment period. When two components are administered “within the same treatment period,” they can be administered in separate compositions according to their own administration schedules, as long as the periods of administration for the two components end around the same day or within a short time period, such as within 1 day, 1 week, or 1 month. “Overlapping administration” refers to administration of the two or more therapies or components not within the same overall treatment period, but with at least one overlapping treatment period. “Subsequent administration” refers to administration of the two or more therapies or components during different treatment periods, one after the other. The use of the term “in combination with” does not restrict the order in which therapies or components are administered to a subject. For example, a first therapy or component can be administered prior to, concomitantly with or simultaneously with, or subsequent to the administration of a second therapy or component. In some embodiments, a first therapy or component (e.g. an RNAi component), a second therapy or component (e.g., a compound of Formula (I)), and a third therapy or component (e.g., an interferon) are administered in the same composition. In other embodiments, a first therapy or component (e.g. an RNAi component), a second therapy or component (e.g., a compound of Formula (I)), and a third therapy or component (e.g., an interferon) are administered in separate compositions, such as two or three separate compositions.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of RNAi component described herein may contain particular components arranged in a particular order, those having ordinary skill in the art will appreciate that the concepts disclosed herein may equally apply to other components arranged in other orders that can be used in RNAi of the application. The application contemplates use of any of the applicable components in any combination that can be used the application, whether or not a particular combination is expressly described. The invention generally relates to a therapeutic combination comprising one or more HBV RNAi, a compound of formula (I) and an interferon.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyl-tartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

Positions indicated on phenyl (e.g. ortho, meta and/or para) are indicated relative to the bond connecting the phenyl to the main structure. An example with regard to the position of para R², location is indicated relative to the nitrogen (*) connected to the main structure:

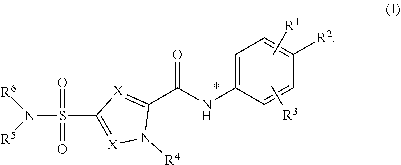

When any variable (e.g. halogen or C₁₋₄alkyl) occurs more than one time in any constituent, each definition is independent.

### Combination

Provided herein is a combination of an effective amount of an RNAi component, an effective amount of a capsid assembly modulator (CAM) of formula (I) or a pharmaceutically acceptable salt thereof, and an effective amount of an interferon.

### CAM Compound

In some embodiments, the CAM is a compound of Formula (I):

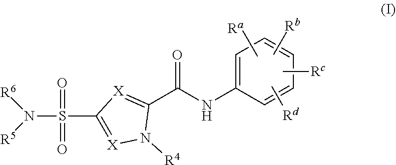

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments of compounds of Formula (I), R^{a}, R^{b} and R^{c} are independently hydrogen, fluoro, bromo, chloro, or CN. In some embodiments of compounds of Formula (I), R⁴ is C₁-C₃ alkyl. In some embodiments of compounds of Formula (I), R⁴ is methyl. In some embodiments of compounds of Formula (I), R⁶ is C₂-C₆ alkyl optionally substituted with one or more of—OH, fluoro, or C₁-C₄ alkyl optionally substituted with R¹⁰. In some embodiments of compounds of Formula (I), R⁶ is C₂-C₆ alkyl substituted with one or more fluoro. In some embodiments of compounds of Formula (I), each R⁷ is independent hydrogen, halogen or methyl. In some embodiments of compounds of Formula (I), at least one R⁷ is hydrogen.

In a further aspect, the COM compound is a compound of Formula (IA)

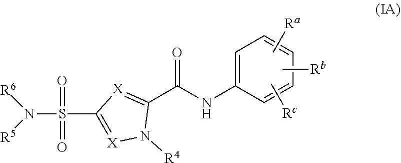

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, C₁-C₃alkyl and C₃-C₄ cycloalkyl;
R⁴ is Hydrogen, C₁-C₃alkyl or C₃-C₄cycloalkyl;
R⁵ is Hydrogen;
R⁶ is selected from the group consisting of C₂-C₆ alkyl, C₁-C₄ alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, —CN, Fluoro, Chloro, Bromo, —CHF₂, —CF₂-methyl, —CH₂F, —CF₃, C₁-C₃alkyl or C₃-C₄cycloalkyl;
R⁸ represents 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In one embodiment, the CAM compound is a compound Formula (IC)

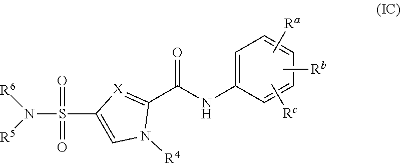

or a stereoisomer or tautomeric form thereof, wherein:
X is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, C₁-C₃alkyl and C₃-C₄cycloalkyl;
R⁴ is Hydrogen, C₁-C₃alkyl or C₃-C₄ cycloalkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄ alkyl optionally substituted with R¹⁰;
R⁷ is hydrogen, —CN, Fluoro, Chloro, Bromo, —CHF₂, —CF₂-methyl, —CH₂F, —CF₃, C₁-C₃alkyl or C₃-C₄cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃; R¹¹ is hydrogen or C₁-C₃alkyl;
or a pharmaceutically acceptable salt or a solvate thereof.

In some embodiments of compounds of Formula (IA) or (IC), R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, and C₁-C₃alkyl. In some embodiments of compounds of Formula (IA) or (IC), R⁴ is Hydrogen, or methyl and R⁵ is Hydrogen. In some embodiments of compounds of Formula (IA) or (IC), R⁶ is selected from the group consisting of C₂-C₆alkyl and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰. In some embodiments of compounds of Formula (IA) or (IC), R⁷ is hydrogen, —CN, Fluoro, Chloro, Bromo, —CHF₂, —CF₂-methyl, —CH₂F, —CF₃ or methyl. In some embodiments of compounds of Formula (IA) or (IC), R⁹ is, C₁-C₄alkyloxy, —SO₂-methyl, —C(═O)—OR¹¹ or —C(═O)—N(R¹¹)₂. In some embodiments of compounds of Formula (IA) or (IC), R¹⁰ is —CN, —OH, Fluoro, —CHF₂, —CH₂F or —CF₃ and R¹¹ is hydrogen or C₁-C₃alkyl.

In some embodiments of compounds of Formula (IA) or (IC), R^{a}, R^{b} and R^{c} are independently hydrogen, fluoro, bromo, chloro, or CN. In some embodiments of compounds of Formula (IA) or (IC), R⁴ is C₁-C₃ alkyl. In some embodiments of compounds of Formula (IA) or (IC), R⁴ is methyl. In some embodiments of compounds of Formula (IA) or (IC), R⁶ is C₂-C₆ alkyl optionally substituted with one or more of—OH, fluoro, or C₁-C₄ alkyl optionally substituted with R¹⁰. In some embodiments of compounds of Formula (IA) or (IC), R⁶ is C₂-C₆ alkyl substituted with one or more fluoro. In some embodiments of compounds of Formula (IA) or (IC), each R⁷ is independent hydrogen, halogen or methyl. In some embodiments of compounds of Formula (IA) or (IC), at least one R⁷ is hydrogen.

Another embodiment of the present invention relates to those compounds of Formula (I), (IA), (IC) or any subgroup thereof as mentioned in any of the other embodiments wherein one or more of the following restrictions apply:
(a) R⁴ is C₁-C₃alkyl, preferably methyl; R⁶ is selected from the group consisting of G-C₆alkyl optionally being substituted with one or more Fluoro; and R⁷ is hydrogen Fluoro, Chloro or C₁-C₃alkyl, preferably hydrogen Fluoro, Chloro or methyl.
(b) R^{b} is Hydrogen or Fluoro.
(c) R^{a} and R^{c} are independently selected from the group consisting of Hydrogen, Fluoro, Chloro —CN and methyl.
(d) R^{b} is Hydrogen or Fluoro and R^{a} and R^{c} are independently selected from the group consisting of Hydrogen, Fluoro, Chloro and —CN.
(e) R⁶ contains a 3-7 membered saturated ring optionally containing one oxygen, more specifically R⁶ is a 4 or 5 membered saturated ring containing one oxygen, such 4 or 5 membered saturated ring optionally substituted with C₁-C₄alkyl optionally substituted with R¹⁰.
(f) R⁶ comprises a branched C₃-C₆alkyl optionally substituted with one or more Fluoro, or wherein R⁶ comprises a C₃-C₆cycloalkyl wherein such C₃-C₆cycloalkyl is substituted with one or more Fluoro or substituted with C₁-C₄alkyl substituted with one or more Fluoro, or wherein R⁶ comprises a C₃-C₆cycloalkyl optionally substituted with one or more Fluoro and/or substituted with C₁-C₄alkyl optionally substituted with one or more Fluoro.
(g) R⁶ comprises a branched C₃-C₆alkyl optionally substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. More specifically, R⁶ is a branched C₃-C₆alkyl substituted with one or more Fluoro.
(h) R⁴ is C₁-C₃alkyl, preferably methyl; R⁶ is selected from the group consisting of C₂-C₆alkyl optionally being substituted with one or more Fluoro; and R⁷ is hydrogen, Fluoro, Chloro or C₁-C₃alkyl, preferably hydrogen Fluoro, Chloro or methyl.

In some embodiments, the compound of Formula (IA) is:

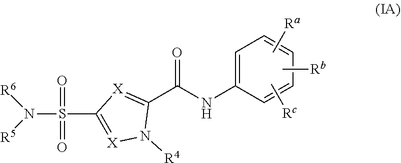

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, CN, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments, the compound of Formula (IA) is:

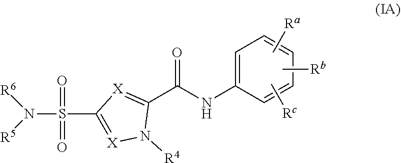

or a stereoisomer or tautomeric form thereof, wherein:

Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In a further aspect, the CAM compound is a compound of Formula (I) is:

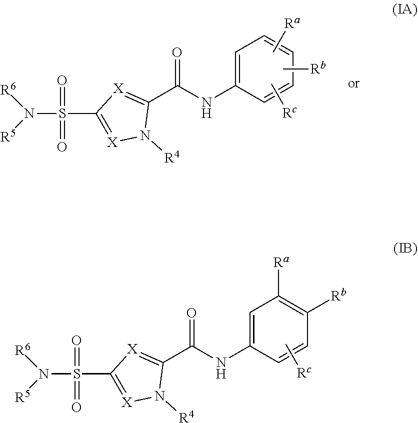

or a stereoisomer or tautomeric form thereof, wherein:

Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments of compounds of Formula (I), (IA), (IB) or (IC), R⁴ is methyl. In some embodiments of compounds of Formula (I), (IA), (IB) or (IC), R^{b} is Hydrogen or Fluoro. In some embodiments of compounds of Formula (I), (IA), (IB) or (IC), R^{a} and R^{c} are independently selected from the group consisting of Hydrogen, Fluoro, Chloro —CN and methyl. Preferably, R^{b} is Hydrogen or Fluoro and R^{a} and R^{c} are independently selected from the group consisting of Hydrogen, Fluoro, Chloro and —CN.

In some embodiments of compounds of Formula (I), (IA), (IB) or (IC), R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰. In some embodiments of compounds of Formula (I), (IA), (IB) or (IC), R⁶ contains a 3-7 membered saturated ring optionally containing one oxygen, more specifically R⁶ is a 5 membered saturated ring containing one oxygen, such 5 membered saturated ring optionally substituted with C₁-C₄alkyl optionally substituted with R¹⁰.

In some embodiments of compounds of Formula (I), (IA), (IB) or (IC), R⁶ comprises a branched C₃-C₆alkyl optionally substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. In some embodiments of compounds of Formula (I), (IA), (IB) or (IC), R⁶ is a branched C₃-C₆alkyl substituted with one or more Fluoro.

In some embodiments of compounds of Formula (I), (IA), (IB) or (IC), R^{a}, R^{b} and R^{c} are independently hydrogen, fluoro, bromo, chloro, or CN. In some embodiments of compounds of Formula (I), (IA), (IB) or (IC), R⁴ is C₁-C₃ alkyl. In some embodiments of compounds of Formula (I), (IA), (IB) or (IC), R⁴ is methyl. In some embodiments of compounds of Formula (I), (IA), (IB) or (IC), R⁶ is C₂-C₆ alkyl optionally substituted with one or more of—OH, fluoro, or C₁-C₄ alkyl optionally substituted with R¹⁰. In some embodiments of compounds of Formula (I), (IA), (IB) or (IC), R⁶ is C₂-C₆ alkyl substituted with one or more fluoro. In some embodiments of compounds of Formula (I), (IA), (IB) or (IC), each R⁷ is independent hydrogen, halogen or methyl. In some embodiments of compounds of Formula (IA) or (IC), at least one R⁷ is hydrogen.

In a further aspect, the CAM compound is a compound of Formula (ID)

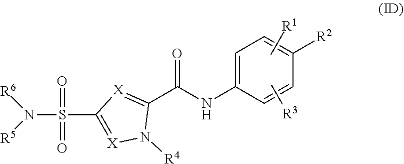

or a stereoisomer or tautomeric form thereof, wherein:

Each X independently is CR⁷;
R² is Hydrogen, CN, Chloro or Fluoro;
R¹ and R³ are independently selected from the group consisting of Hydrogen, Fluoro, Bromo, Chloro, —CHF₂, —CH₂F, —CF₃, —OCF₃, —CN and methyl, wherein at maximum one of R¹ R² and R³ is Hydrogen if one of R¹ and R³ is Chloro or —OCF₃;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments, the compound of Formula (ID) is:

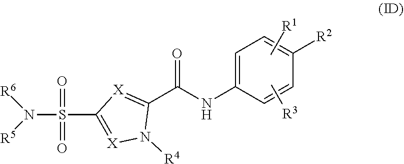

or a stereoisomer or tautomeric form thereof, wherein:

Each X independently is CR⁷;
R² is Hydrogen, CN, or Fluoro;
R¹ and R³ are independently selected from the group consisting of Hydrogen, Fluoro, Bromo, Chloro, —CHF₂, —CH₂F, —CF₃, —CN and methyl, wherein at maximum one of
R¹, R² and R³ is Hydrogen if one of R¹ and R³ is Chloro;
R⁴ is Hydrogen or C₁-C₃alkyl;
R⁵ is Hydrogen;
R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is —C(═O)—OR¹¹ or —C(═O)—N(R¹¹)₂;
R¹⁰ is —CN, —OH, Fluoro, —CHF₂, —CH₂F or —CF₃; and
R¹¹ is hydrogen or C₁-C₃alkyl.

In some embodiments, the compound of Formula (ID) is:

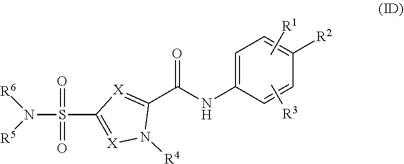

or a stereoisomer or tautomeric form thereof, wherein:

Each X independently is CR⁷;
R² is Hydrogen or Fluoro;
R¹ and R³ are independently selected from the group consisting of Hydrogen, Fluoro, Bromo, Chloro, CHF₂, CH₂F, CF₃ and methyl, wherein at maximum one of R¹, R² and R³ is Hydrogen;
R⁴ is Hydrogen or methyl;
R⁵ is Hydrogen;
R⁶ is selected from the group consisting of C₂-C₆alkyl, C₁-C₃alkyl-R⁸ and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, OH, Fluoro, and C₁-C₄alkyl;
R⁷ is Hydrogen, methyl, Fluoro or Chloro;
R⁸ is 3-7 membered saturated ring optionally containing one or more heteroatoms each independently selected from the group consisting of O, S and N.

In some embodiments of compounds of Formula (ID), at least one X is CH. In some embodiments of compounds of Formula (ID), R^{a}, R^{b} and R^{c} are independently hydrogen, fluoro, bromo, chloro, or CN. In some embodiments of compounds of Formula (ID), R⁴ is C₁-C₃ alkyl. In some embodiments of compounds of Formula (ID), R⁴ is methyl. In some embodiments of compounds of Formula (ID), R⁶ is C₂-C₆ alkyl optionally substituted with one or more of—OH, fluoro, or C₁-C₄ alkyl optionally substituted with R¹⁰. In some embodiments of compounds of Formula (ID), R⁶ is C₂-C₆ alkyl substituted with one or more fluoro. In some embodiments of compounds of Formula (ID), each R⁷ is independent hydrogen, halogen or methyl. In some embodiments of compounds of Formula (ID), at least one R⁷ is hydrogen.

In another aspect, the CAM compound is a compound of Formula (Ia):

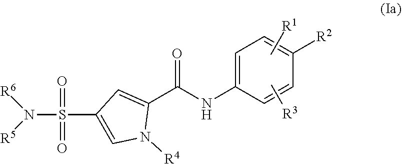

or a stereoisomer or tautomeric form thereof, wherein:
R² is Hydrogen, CN or Fluoro;
R¹ and R³ are independently selected from the group consisting of Hydrogen, Fluoro, Bromo, Chloro, —CHF₂, —CH₂F, —CF₃, —CN and methyl, wherein at maximum one of R¹, R² and R³ is Hydrogen if one of R¹ and R³ is Chloro;
R⁴ is Hydrogen or C₁-C₃alkyl;
R⁵ is Hydrogen;
R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is —C(═O)—OR¹¹ or —C(═O)—N(R¹¹)₂
R¹⁰ is —CN, —OH, Fluoro, —CHF₂, —CH₂F or —CF₃;
R¹¹ is hydrogen or C₁-C₃alkyl;
or a pharmaceutically acceptable salts or a solvate thereof.

In some embodiments, compounds of Formula (ID) are:

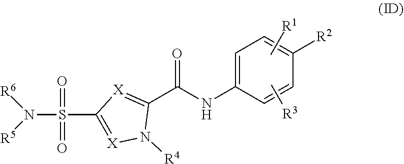

or a stereoisomer or tautomeric form thereof, wherein:
R² is Hydrogen or Fluoro;
R¹ and R³ are independently selected from the group consisting of Hydrogen, Fluoro, CHF₂, CH₂F, CF₃ and methyl, wherein at maximum one of R¹, R² and R³ is Hydrogen;
R⁴ is Hydrogen or methyl;
R⁵ is Hydrogen;
R⁶ is selected from the group consisting of C₂-C₆ alkyl and a 3-7 membered saturated ring optionally containing one or more heteroatoms each independently selected from the group consisting of O, S and N, such 3-7 membered saturated ring or C₁-C₆alkyl optionally being substituted with one or more substituents each independently selected from the group consisting of Hydrogen, OH and C₁-C₄ alkyl.

In another aspect, the CAM compound is a compound of Formula (II):

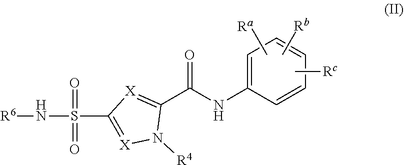

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

In some embodiments of compounds of Formula (II), R^{a}, R^{b} and R^{c} are independently hydrogen, fluoro, bromo, chloro, or CN. In some embodiments of compounds of Formula (II), R⁴ is C₁-C₃ alkyl. In some embodiments of compounds of Formula (II), R⁴ is methyl. In some embodiments of compounds of Formula (II), R⁶ is C₂-C₆ alkyl optionally substituted with one or more of—OH, fluoro, or C₁-C₄ alkyl optionally substituted with R¹⁰. In some embodiments of compounds of Formula (II), R⁶ is CVG, alkyl substituted with one or more fluoro. In some embodiments of compounds of Formula (II), each R⁷ is independent hydrogen, halogen or methyl. In some embodiments of compounds of Formula (II), at least one R⁷ is hydrogen.

In some embodiments, the compound of Formula (ID) is a compound of Formula (III):

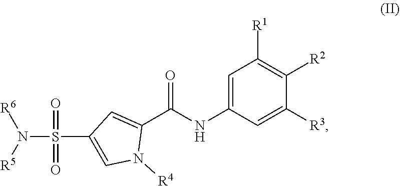

wherein R¹-R⁶ are as defined in Formula (ID).

In some embodiments, the compound of Formula (ID) is a compound of Formula (IV):

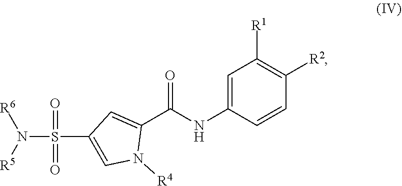

wherein R¹, R², R⁴-R⁶ are as defined in Formula (ID).

In some embodiments of compounds of Formula (III) and (IV), R² is Hydrogen, CN or Fluoro. In some embodiments of compounds of Formula (III) and (IV), R¹ is independently selected from the group consisting of Fluoro, Bromo, Chloro, —CHF₂, —CH₂F, —CF₃, —CN and methyl, wherein if R¹ is Chloro, R² is not Hydrogen. In some embodiments of compounds of Formula (III) and (IV), R⁴ is Hydrogen or C₁-C₃alkyl; R⁵ is Hydrogen; and R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰. In some embodiments of compounds of Formula (III) and (IV), R⁷ is methyl, Fluoro or Chloro. In some embodiments of compounds of Formula (III) and (IV), R⁸ is 3-7 membered saturated ring optionally containing one or more heteroatoms each independently selected from the group consisting of O, S and N. In some embodiments of compounds of Formula (III) and (IV), R⁹ is —C(═O)—OR¹¹ or —C(═O)—N(R¹¹)₂. In some embodiments of compounds of Formula (III) and (IV), R¹⁰ is —CN, Fluoro, —CHF₂, —CH₂F or —CF₃. In some embodiments of compounds of Formula (III) and (IV), R¹¹ is hydrogen.

In some embodiments of compounds of Formula (I)-(IV), (Ia), (IB), (IC) or (ID), R¹ is selected from either Bromo, Chloro, Fluoro or methyl, or Fluoro or methyl. In some embodiments of compounds of Formula (I)-(IV), (Ia), (IB), (IC) or (ID), R¹ is selected from either Fluoro or methyl and at least one of R¹ and R³ is Fluoro. In some embodiments of compounds of Formula (I)-(IV), (Ia), (IB), (IC) or (ID), R¹ is selected from either Fluoro or methyl and at least one of R¹ and R³ is Fluoro, and the other R¹ or R³ is selected from methyl, Fluoro, CHF₂, CH₂F, CF₃ and methyl. In some embodiments of compounds of Formula (I)-(IV), (Ia), (IB), (IC) or (ID), at least two of R¹, R² and R³ are halogens, preferably Bromo, Fluoro or Chloro, even more preferably Fluoro or Chloro. In some embodiments of compounds of Formula (I)-(IV), (Ia), (IB), (IC) or (ID), each of R¹, R² and R³ are halogen, preferably Bromo, Fluoro or Chloro, even more preferably Fluoro or Chloro.

In some embodiments of compounds of Formula (I)-(IV), (Ia), (IB), (IC) or (ID), R⁴ is methyl or ethyl, preferably methyl. In some embodiments of compounds of Formula (I)-(IV), (Ia), (IB), (IC) or (ID), R⁶ contains a 3-7 membered saturated ring optionally containing one oxygen, preferably R⁶ is a 5 membered saturated ring containing one oxygen. In some embodiments of compounds of Formula (I)-(IV), (Ia), (IB), (IC) or (ID), R⁶ comprises a C₁-C₄alkyl substituted with one or more Fluoro. In some embodiments of compounds of Formula (I)-(IV), (Ia), (IB), (IC) or (ID), R⁶ comprises a branched C₃-C₆alkyl substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. In some embodiments of compounds of Formula (I)-(IV), (Ia), (IB), (IC) or (ID), R⁶ comprises a carbon atom without hydrogen substituent. Preferably, carbon without hydrogen substituent is directly attached to the Nitrogen of the —N—SO₂˜ moiety.

In some embodiments of compounds of Formula (I)-(IV), (Ia), (IB), (IC) or (ID), R^{a}, R^{b} and R^{c} are independently hydrogen, fluoro, bromo, chloro, or CN. In some embodiments of compounds of Formula (I)-(IV), (Ia), (IB), (IC) or (ID), R⁴ is C₁-C₃ alkyl. In some embodiments of compounds of Formula (I)-(IV), (Ia), (IB), (IC) or (ID), R⁴ is methyl. In some embodiments of compounds of Formula (I)-(IV), (Ia), (IB), (IC) or (ID), R⁶ is C₂-C₆ alkyl optionally substituted with one or more of—OH, fluoro, or C₁-C₄ alkyl optionally substituted with R¹⁰. In some embodiments of compounds of Formula (I)-(IV), (Ia), (IB), (IC) or (ID), R⁶ is C₂-C₆ alkyl substituted with one or more fluoro. In some embodiments of compounds of Formula (I)-(IV), (Ia), (IB), (IC) or (ID), each R⁷ is independent hydrogen, halogen or methyl. In some embodiments of compounds of Formula (I)-(IV), (Ia), (IB), (IC) or (ID), at least one R⁷ is hydrogen.

Further combinations of any of the embodiments are also envisioned to be in the scope of the present invention.

In another aspect, the CAM compound is selected from the group consisting of:

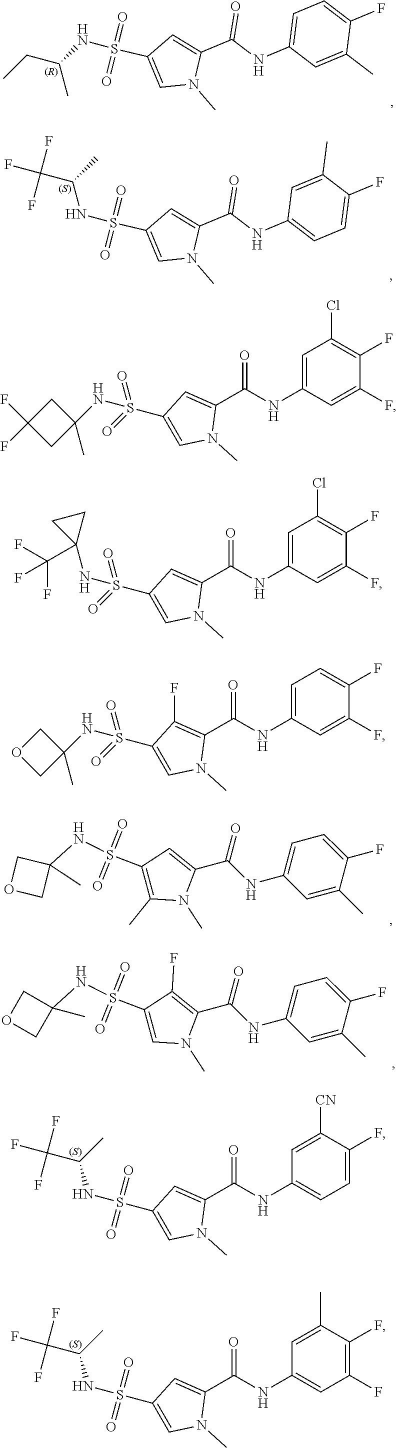

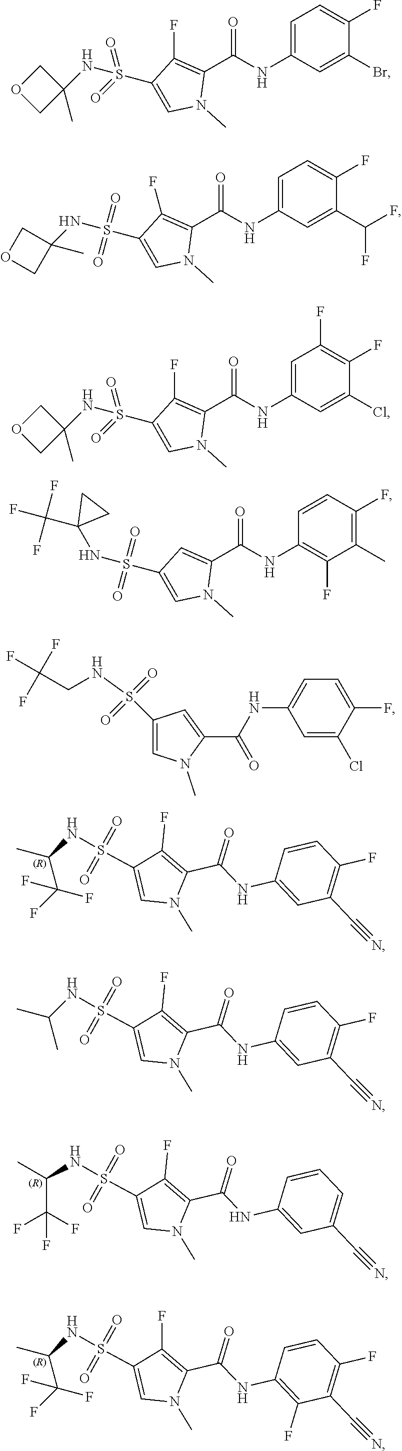

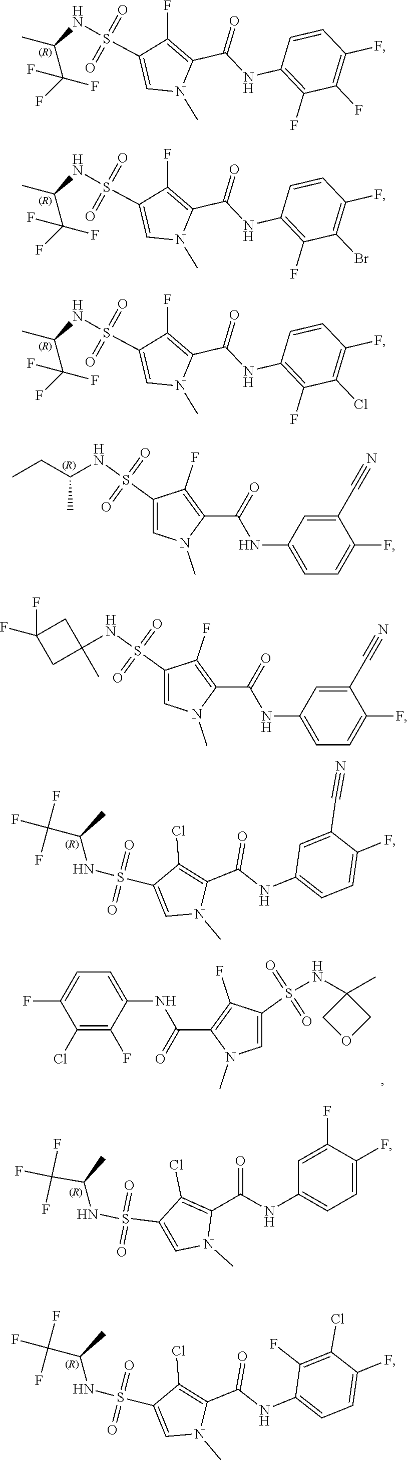

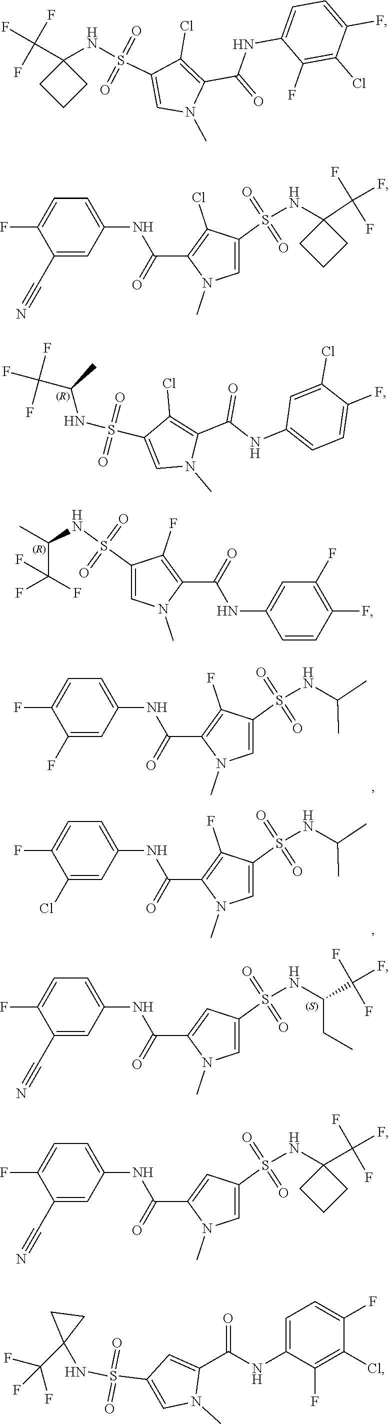

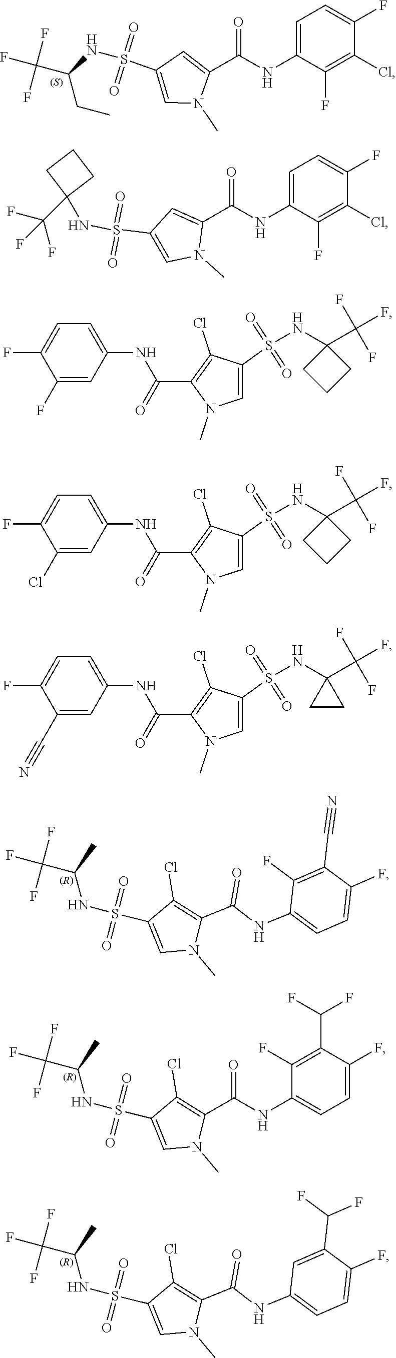

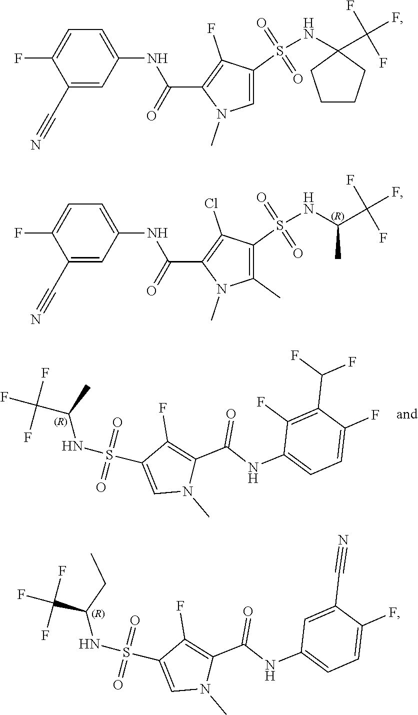

or a pharmaceutically acceptable salt of any of the foregoing.

In one embodiment, the CAM compound is

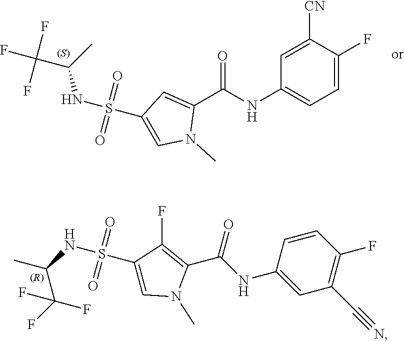

or a pharmaceutically acceptable salt thereof.

In another aspect, the CAM compound of the present disclosure or any subgroup thereof can be in the form of a pharmaceutically acceptable salt or a solvate. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethyl amine, diethylamine, tri ethyl amine, tripropylamine, and ethanolamine. In some embodiments, the pharmaceutically acceptable base addition salt is selected from ammonium, potassium, sodium, calcium, and magnesium salts.

### RNAi Component

In one aspect, the RNAi component comprises one or more RNAi agents. Each RNAi agent disclosed herein includes at least a sense strand and an antisense strand. The sense strand and the antisense strand can be partially, substantially, or fully complementary to each other. The length of the RNAi agent sense and antisense strands described herein each can be 16 to 30 nucleotides in length. In some embodiments, the sense and antisense strands are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The HBV RNAi agents disclosed herein have been designed to include antisense strand sequences that are at least partially complementary to a sequence in the HBV genome that is conserved across the majority of known serotypes of HBV. The RNAi agents described herein, upon delivery to a cell expressing HBV, inhibit the expression of one or more HBV genes in vivo or in vitro.

An RNAi agent includes a sense strand (also referred to as a passenger strand) that includes a first sequence, and an antisense strand (also referred to as a guide strand) that includes a second sequence. A sense strand of the HBV RNAi agents described herein includes a core stretch having at least about 85% identity to a nucleotide sequence of at least 16 consecutive nucleotides in an HBV mRNA. In some embodiments, the sense strand core nucleotide stretch having at least about 85% identity to a sequence in an HBV mRNA is 16, 17, 18, 19, 20, 21, 22, or 23 nucleotides in length. An antisense strand of an HBV RNAi agent comprises a nucleotide sequence having at least about 85% complementary over a core stretch of at least 16 consecutive nucleotides to a sequence in an HBV mRNA and the corresponding sense strand. In some embodiments, the antisense strand core nucleotide sequence having at least about 85% complementarity to a sequence in an HBV mRNA or the corresponding sense strand is 16, 17, 18, 19, 20, 21, 22, or 23 nucleotides in length.

In some embodiments, the RNAi component comprises a first RNAi agent comprising an antisense strand comprising a nucleotide sequence of any one of the following: SEQ ID NOT, SEQ ID NO:2, SEQ ID NOT, SEQ ID NO: 4, SEQ ID NO: 5, SEQ ID NO:6, and SEQ ID NOT, and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15, or a second RNAi agent comprising an antisense strand comprising a nucleotide sequence of any one of the following: SEQ ID NOT and SEQ ID NOV, and a sense strand comprising a nucleotide sequence of any one of the following: SEQ ID NO: 16, SEQ ID NO: 17, SEQ ID NO: 18, and SEQ ID NO: 19. In some embodiments, the RNAi component comprises a first RNAi agent comprising an antisense strand comprising a nucleotide sequence of any one of the following: SEQ ID NO:1, SEQ ID NO:2, SEQ ID NO:3, SEQ ID NO: 4, SEQ ID NO: 5, SEQ ID NO:6, and SEQ ID NO:7, and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15, and a second RNAi agent comprising an antisense strand comprising a nucleotide sequence of any one of the following: SEQ ID NO:8 and SEQ ID NO:9, and a sense strand comprising a nucleotide sequence of any one of the following: SEQ ID NO: 16, SEQ ID NO: 17, SEQ ID NO: 18, and SEQ ID NO: 19.

In some embodiments, the first and the second RNAi agents disclosed herein comprise any of the sequences in Table 1.

TABLE 1
Exemplary sequences for first and second RNAi agents

| Antisense | | Sense | |
|---|---|---|---|
| SEQ | | SEQ | |
| ID | Unmodified | ID | Unmodified |
| NO | sequence (5′→3′) | NO | sequence (5′→3′) |
| 5 | AGAAAAUUGAGAGAAGUCCAC | 14 | GUGGACUUCUCUCAAUUUUCU |
| 6 | AGAAAAUUGAGAGAAGUCCACUU | 14 | GUGGACUUCUCUCAAUUUUCU |
| 7 | AGAAAAUUGAGAGAAGUCCACC | 15 | GGUGGACUUCUCUCAAUUUUCU |
| 9 | UACCAAUUUAUGCCUACAGCG | 19 | CGCUGUAGGCAUAAAUUGGUA |

### Targeting Group

In some embodiments, the RNAi agents are delivered to target cells or tissues using any oligonucleotide delivery technology known in the art. Nucleic acid delivery methods include, but are not limited to, by encapsulation in liposomes, by iontophoresis, or by incorporation into other vehicles, such as hydrogels, cyclodextrins, biodegradable nanocapsules, and bioadhesive microspheres, proteinaceous vectors or Dynamic Polyconjugates (DPCs) (see, for example WO 2000/053722, WO 2008/022309, WO 2011/104169, and WO 2012/083185, each of which is incorporated herein by reference). In some embodiments, an HBV RNAi agent is delivered to target cells or tissues by covalently linking the RNAi agent to a targeting group. In some embodiments, the targeting group can include a cell receptor ligand, such as an asialoglycoprotein receptor (ASGPr) ligand. In some embodiments, an ASGPr ligand includes or consists of a galactose derivative cluster. In some embodiments, a galactose derivative cluster includes an N-acetyl-galactosamine trimer or an N-acetyl-galactosamine tetramer. In some embodiments, a galactose derivative cluster is an N-acetyl-galactosamine trimer or an N-acetyl-galactosamine tetramer.

A targeting group can be linked to the 3′ or 5′ end of a sense strand or an antisense strand of an HBV RNAi agent. In some embodiments, a targeting group is linked to the 3′ or 5′ end of the sense strand. In some embodiments, a targeting group is linked to the 5′ end of the sense strand. In some embodiments, a targeting group is linked to the RNAi agent via a linker.

In some embodiments, the RNAi component comprises a combination or cocktail of a first and a second RNAi agents having different nucleotide sequences. In some embodiments, the first and the second RNAi agents are each separately and independently linked to targeting groups. In some embodiments, the first and the second RNAi agents are each linked to targeting groups comprised of N-acetyl-galactosamines. In some embodiments, when first and the second RNAi agents are included in a composition, each of the RNAi agents is linked to the same targeting group. In some embodiments, when first and the second RNAi agents are included in a composition, each of the RNAi agents is linked to different targeting groups, such as targeting groups having different chemical structures.

In some embodiments, targeting groups are linked to the first and the second RNAi agents without the use of an additional linker. In some embodiments, the targeting group is designed having a linker readily present to facilitate the linkage to the first or the second RNAi agent. In some embodiments, when the first and the second RNAi agents are included in a composition, the first and the second RNAi agents may be linked to the targeting groups using the same linkers. In some embodiments, when the first and the second RNAi agents are included in a composition, the first and the second RNAi agents are linked to the targeting groups using different linkers.

Examples of targeting groups and linking groups are provided in Table 2. The non-nucleotide group can be covalently linked to the 3′ and/or 5′ end of either the sense strand and/or the antisense strand. In some embodiments, the first or second RNAi agent contains a non-nucleotide group linked to the 3′ and/or 5′ end of the sense strand. In some embodiments, a non-nucleotide group is linked to the 5′ end of the first or second RNAi agent sense strand. A non-nucleotide group may be linked directly or indirectly to the first or second RNAi agent via a linker/linking group. In some embodiments, a non-nucleotide group is linked to the first or second RNAi agent via a labile, cleavable, or reversible bond or linker.

Targeting groups and linking groups include the following, for which their chemical structures are provided below in Table 2: (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s. Each sense strand and/or antisense strand can have any targeting groups or linking groups listed above, as well as other targeting or linking groups, conjugated to the 5′ and/or 3′ end of the sequence.

TABLE 2
Structures Representing Various Modified Nucleotides, Targeting Groups, And Linking Groups

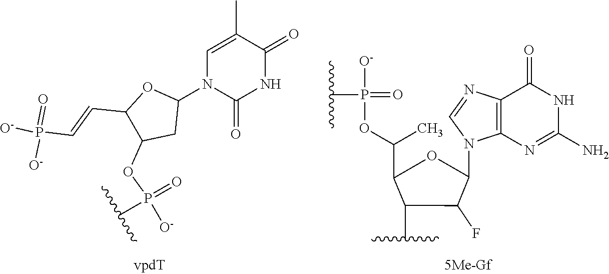 [SMILES: *P(=O)([O-])OC1CC(n2cc(C)c(=O)[nH]c2=O)OC1/C=C/P(=O)([O-])[O-] ; *C1C(F)C(n2cnc3c(=O)[nH]c(N)nc32)OC1C(C)OP(*)(=O)[O-]]

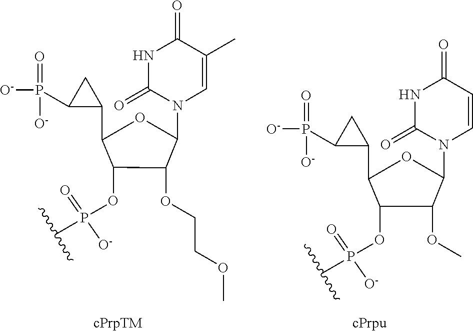 [SMILES: *P(=O)([O-])OC1C(C2CC2P(=O)([O-])[O-])OC(n2cc(C)c(=O)[nH]c2=O)C1OCCOC ; *P(=O)([O-])OC1C(C2CC2P(=O)([O-])[O-])OC(n2ccc(=O)[nH]c2=O)C1OC]

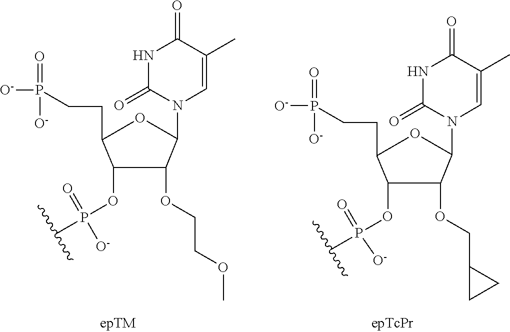 [SMILES: *P(=O)([O-])OC1C(CCP(=O)([O-])[O-])OC(n2cc(C)c(=O)[nH]c2=O)C1OCCOC ; *P(=O)([O-])OC1C(CCP(=O)([O-])[O-])OC(n2cc(C)c(=O)[nH]c2=O)C1OCC1CC1]

When positioned internally on oligonucleotide:

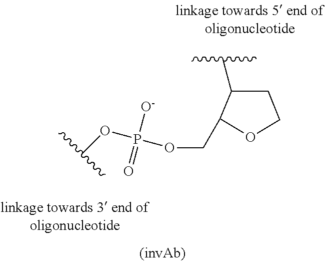 [SMILES: *OP(=O)([O-])OCC1OCCC1*]

When positioned internally on oligonucleotide:

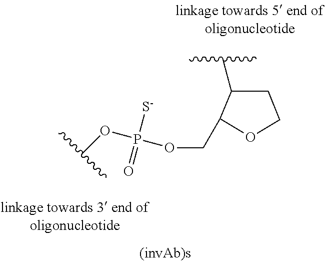 [SMILES: *OP(=O)([S-])OCC1OCCC1*]

When positioned at the 3′ terminal end of oligonucleotide:

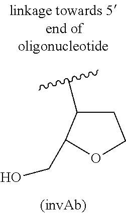 [SMILES: *C1CCOC1CO]

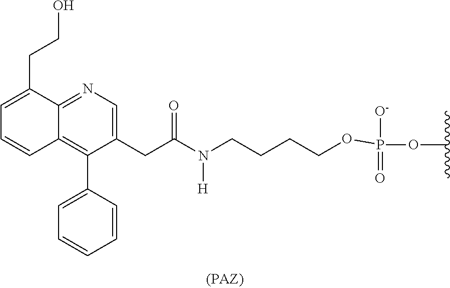 [SMILES: *OP(=O)([O-])OCCCCNC(=O)Cc1cnc2c(CCO)cccc2c1-c1ccccc1]

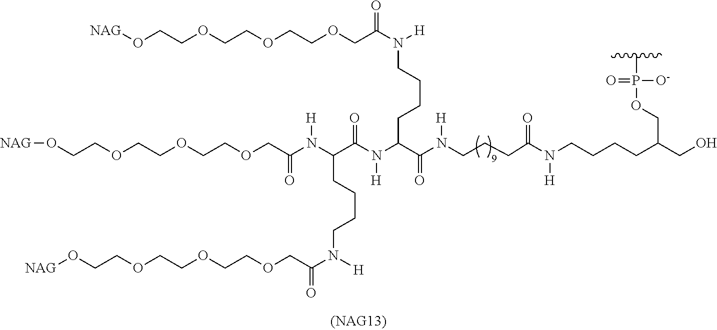

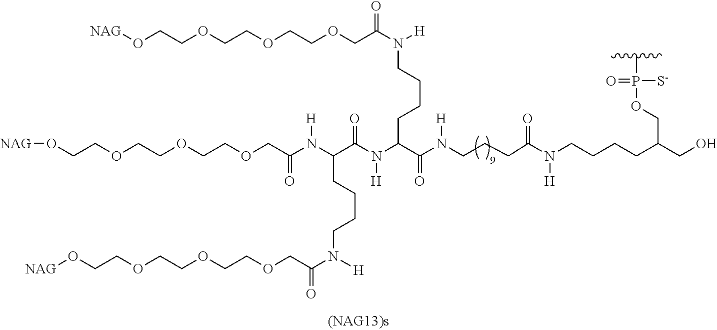

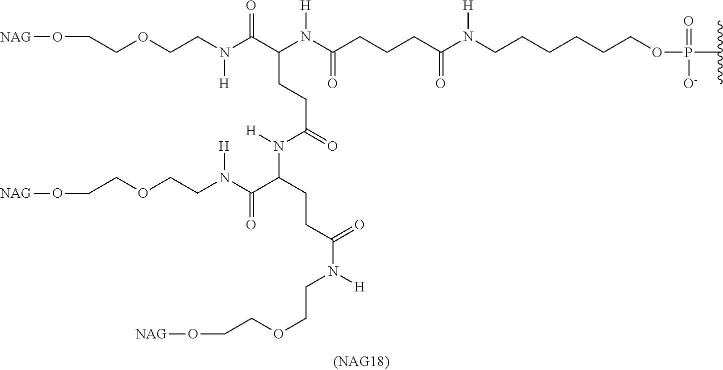

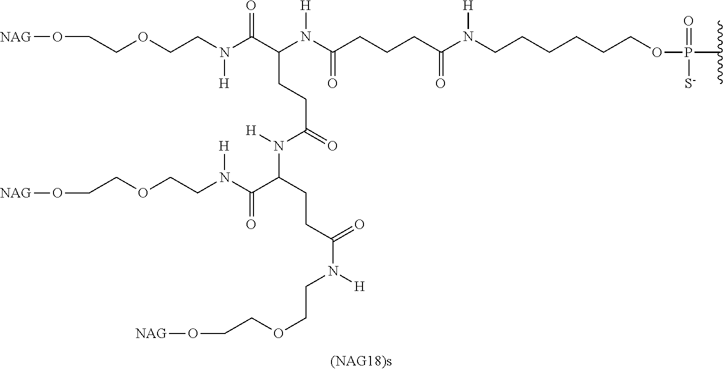

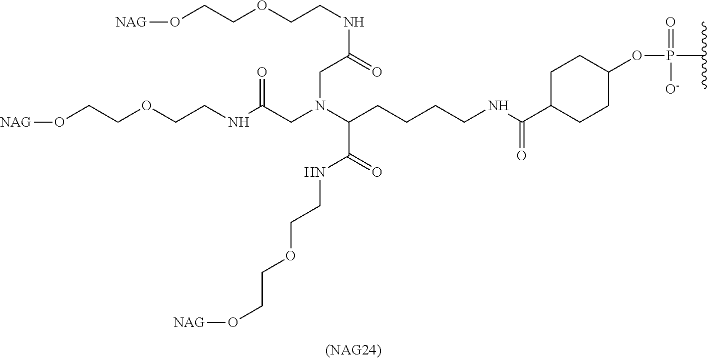

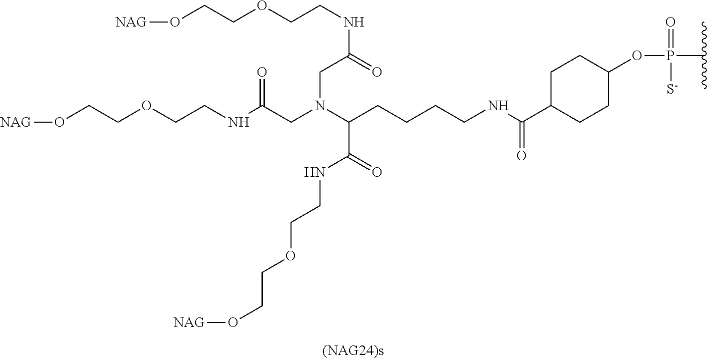

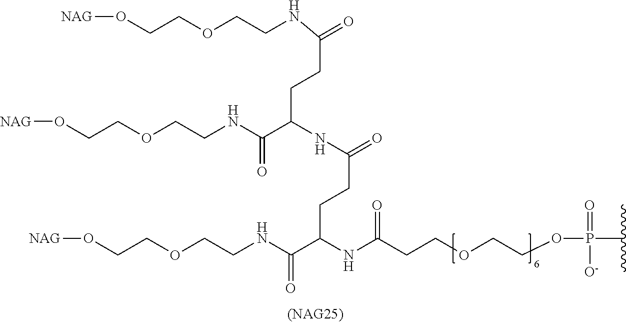

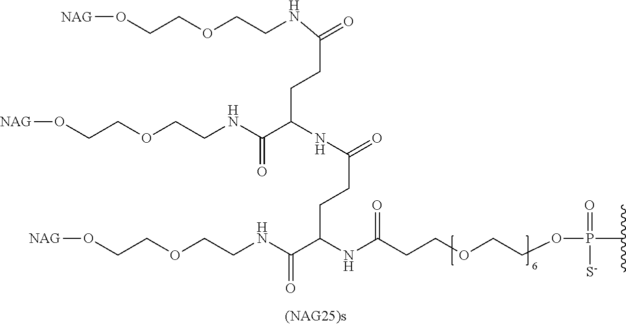

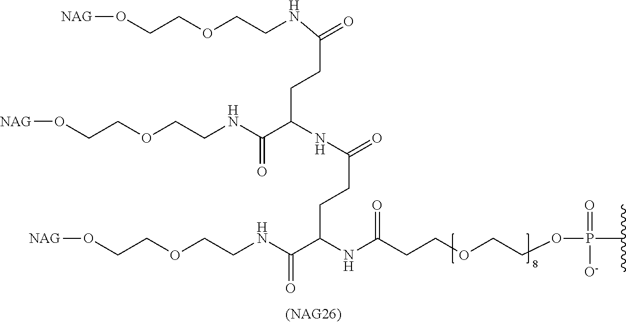

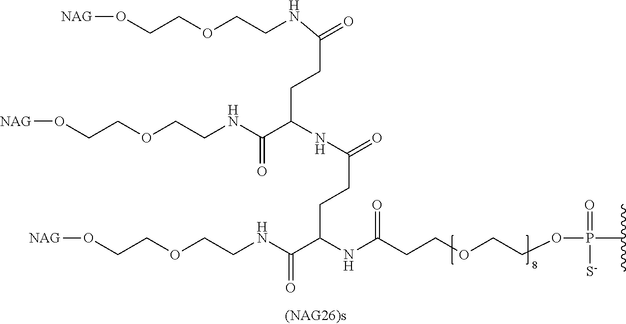

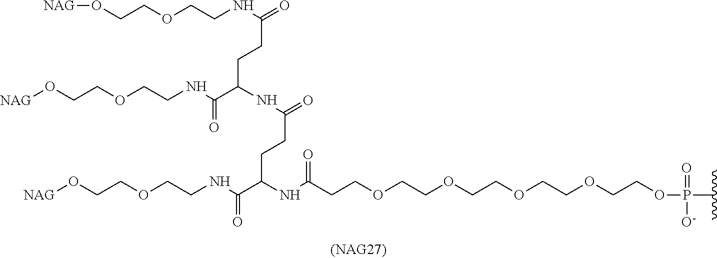

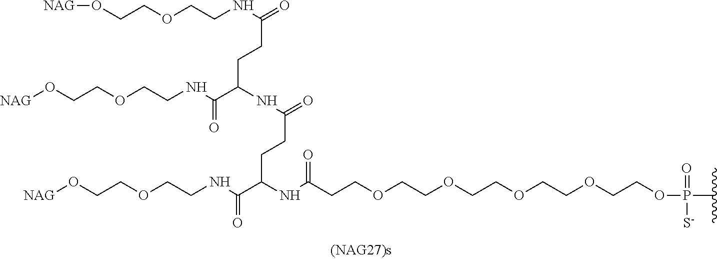

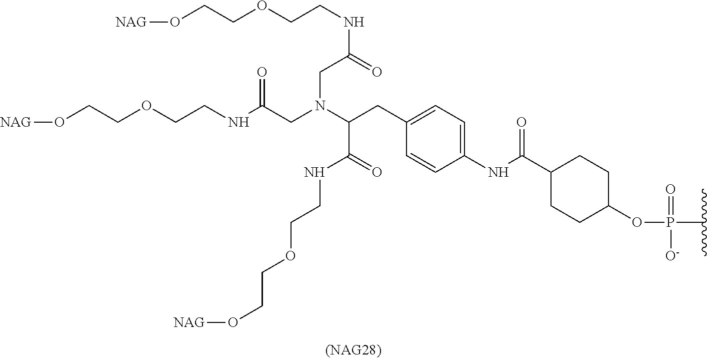

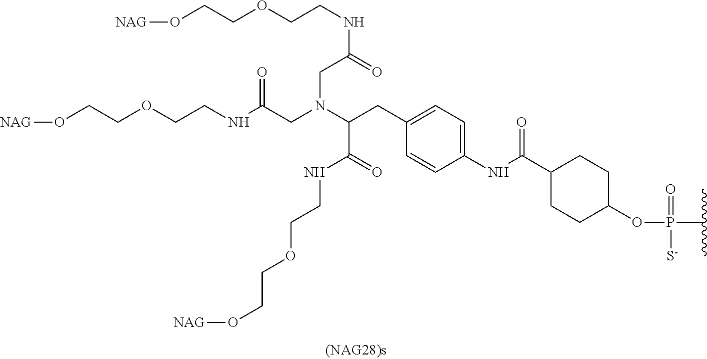

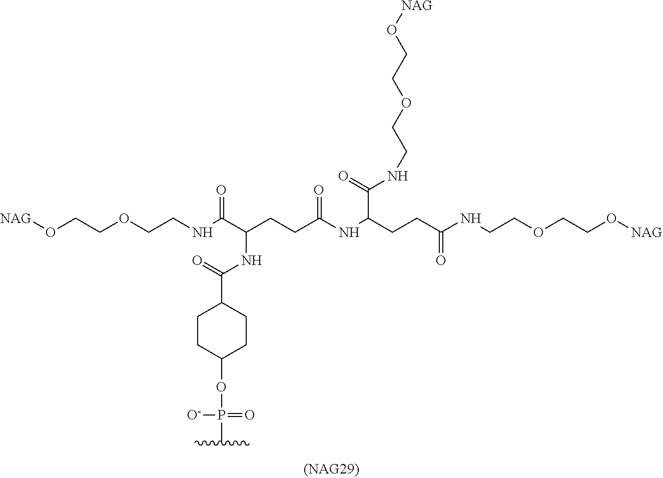

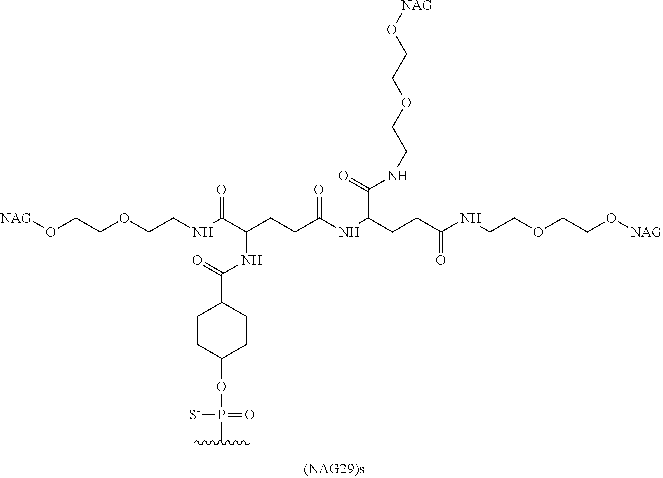

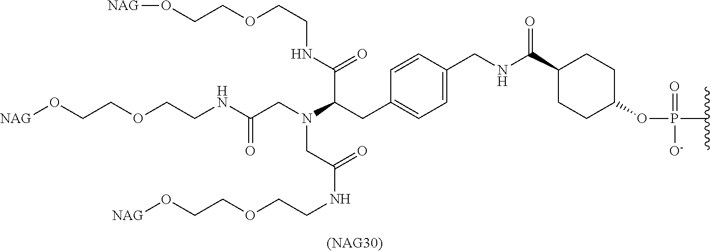

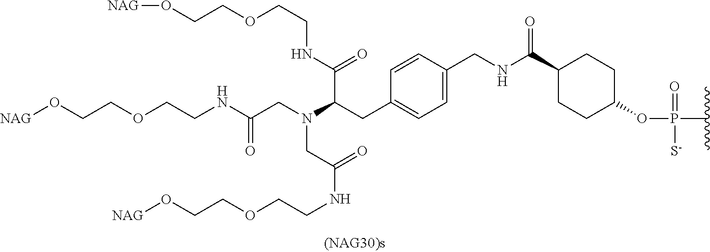

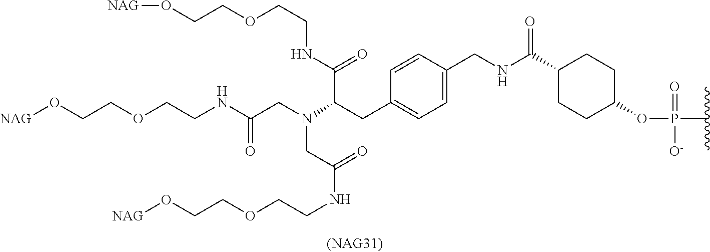

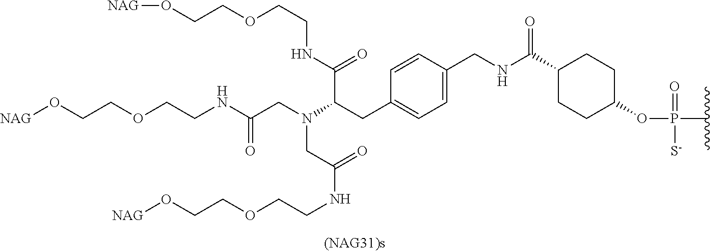

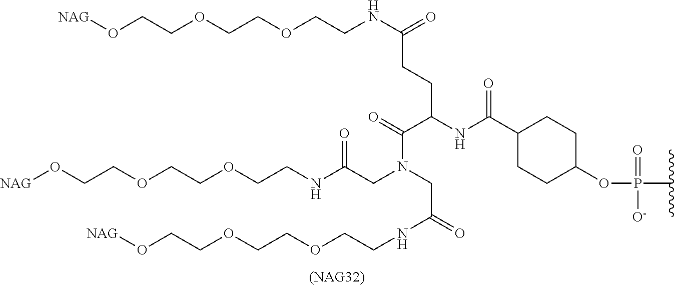

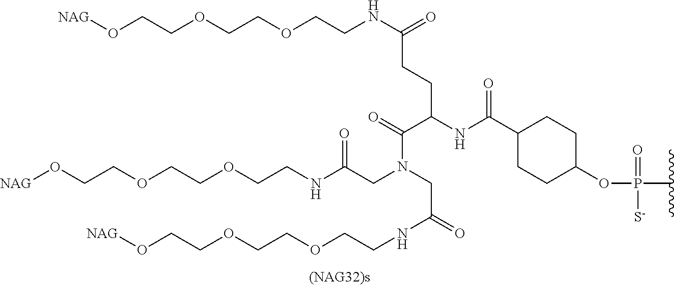

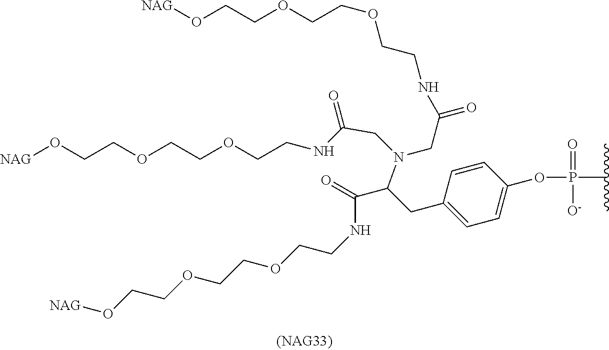

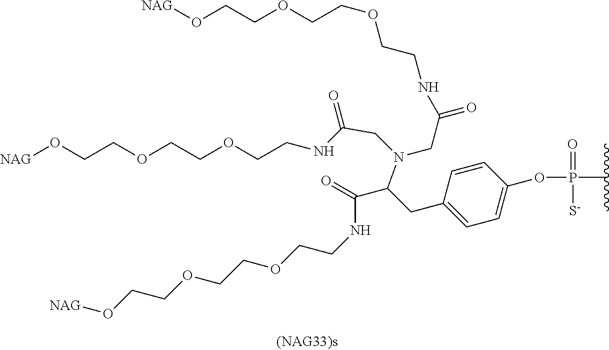

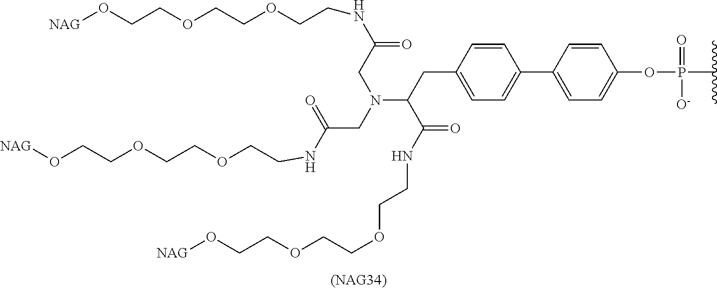

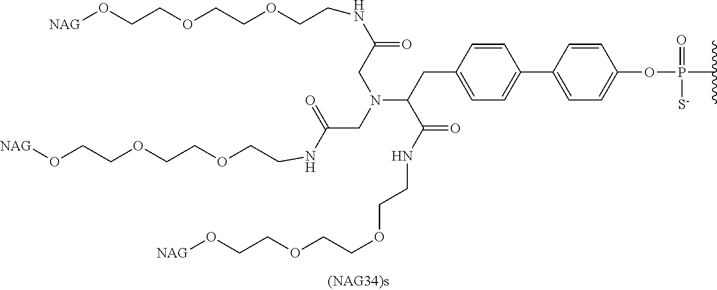

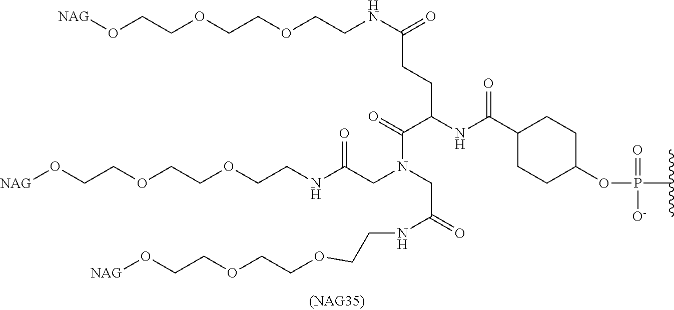

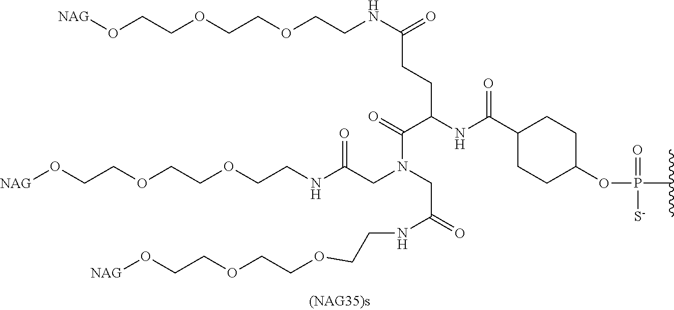

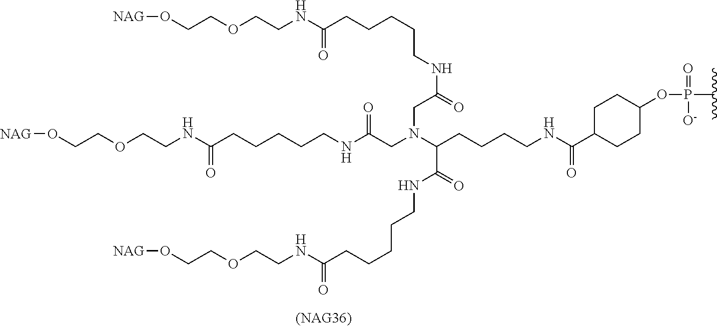

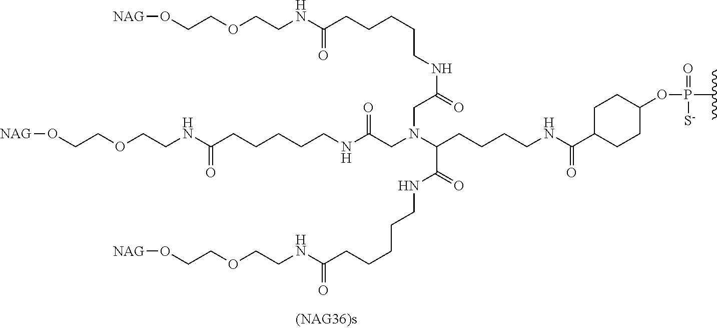

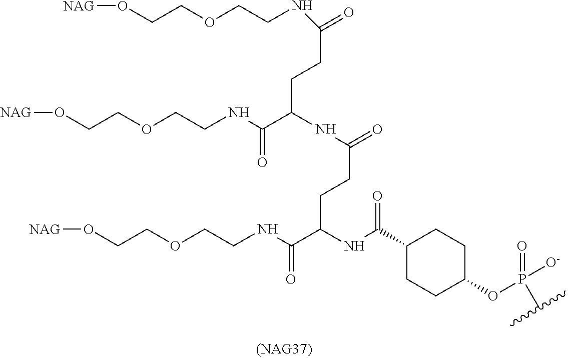

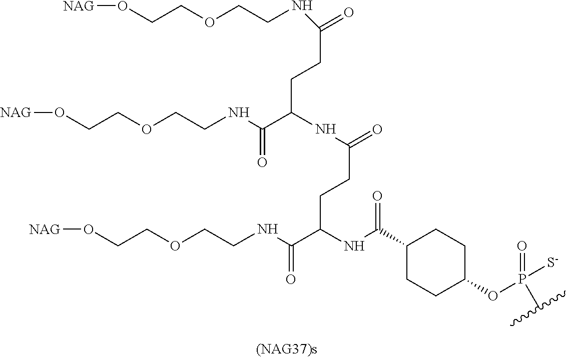

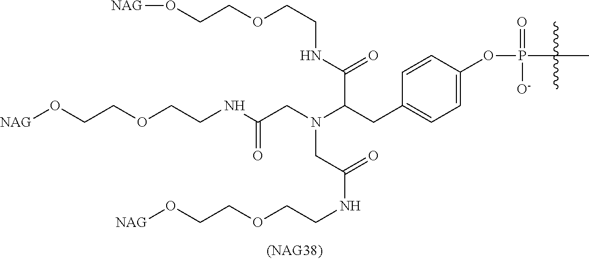

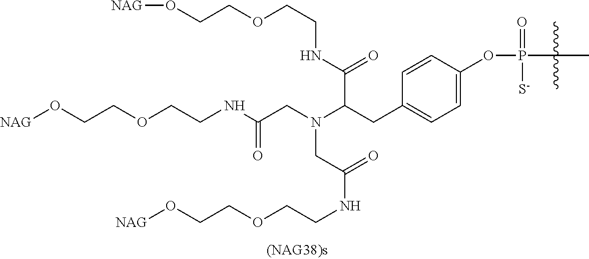

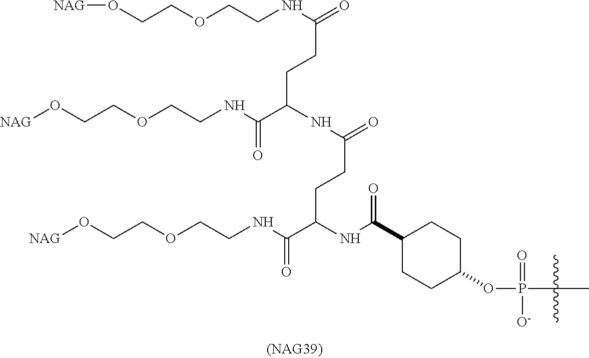

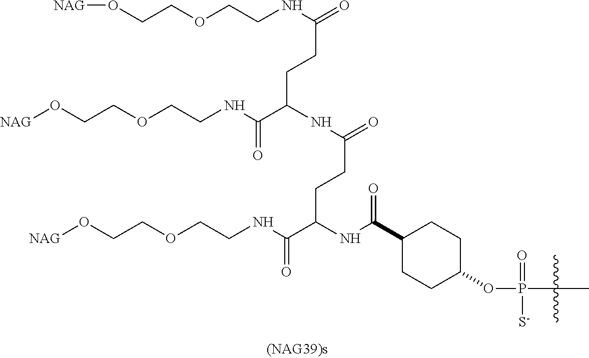

### Modified Nucleotides

In some embodiments, the first or the second RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn, invAb), non-natural base-comprising nucleotides, bridged nucleotides, peptide nucleic acids (PNAs), 2′, 3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification may be incorporated in the first or second RNAi agent or even in a single nucleotide thereof. The RNAi agent sense strands and antisense strands may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of the first or the second RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides. As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

### Modified Internucleoside Linkages

In some embodiments, one or more nucleotides of the first or the second RNAi agent are linked by non-standard linkages or backbones (i.e., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of the first or the second RNAi agent can contain 1, 2, 3, 4, 5, or 6 phosphorothioate linkages, an antisense strand of the first or the second RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of the first or the second RNAi agent can contain 1, 2, 3, or 4 phosphorothioate linkages, an antisense strand of the first or the second RNAi agent can contain 1, 2, 3, or 4 phosphorothioate linkages, or both the sense strand and the antisense strand independently can contain 1, 2, 3, or 4 phosphorothioate linkages.

In some embodiments, the first or the second RNAi agent sense strand contains at least two phosphorothioate internucleoside linkages. In some embodiments, at least two phosphorothioate internucleoside linkages are between the nucleotides at positions 1-3 from the 3′ end of the sense strand. In some embodiments, at least two phosphorothioate internucleoside linkages are between the nucleotides at positions 1-3, 2-4, 3-5, 4-6, 4-5, or 6-8 from the 5′ end of the sense strand. In some embodiments, the first or the second RNAi agent antisense strand contains four phosphorothioate internucleoside linkages. In some embodiments, the four phosphorothioate internucleoside linkages are between the nucleotides at positions 1-3 from the 5′ end of the sense strand and between the nucleotides at positions 19-21, 20-22, 21-23, 22-24, 23-25, or 24-26 from the 5′ end. In some embodiments, the first or the second RNAi agent contains at least two phosphorothioate internucleoside linkages in the sense strand and three or four phosphorothioate internucleoside linkages in the antisense strand.

In some embodiments, the first or the second RNAi agent contains one or more modified nucleotides and one or more modified internucleoside linkages. In some embodiments, a 2′-modified nucleoside is combined with a modified internucleoside linkage.

In some embodiments, the first and the second RNAi agents disclosed herein comprise any of the modified sequences in Table 3.

TABLE 3
Exemplary modified sequences for first and second RNAi agents

| Antisense | | Sense | |
|---|---|---|---|
| SEQ | | SEQ | |
| ID | | ID | |
| NO | modified sequence (5′→3′) | NO | modified sequence (5′→3′) |
| 1 | asGfsasAfaAfuugagAfgAfaGfuCfcAfsc | 10 | (NAG25)sgsuggacuuCfUfCfucaauuuucus(invAb) |
| 1 | asGfsasAfaAfuugagAfgAfaGfuCfcAfsc | 11 | (NAG37)s(invAb)sguggacuuCfUfCfucaauuuucus(invAb) |
| 1 | asGfsasAfaAfuugagAfgAfaGfuCfcAfsc | 13 | (NAG25)s(invAb)sguggacuuCfUfCfucaauuuucus(invAb) |
| 2 | asGfsasAfaAfuUfgAfgAfgAfaGfuCfcasc | 10 | (NAG25)sgsuggacuuCfUfCfucaauuuucus(invAb) |
| 2 | asGfsasAfaAfuUfgAfgAfgAfaGfuCfcasc | 11 | (NAG37)s(invAb)sguggacuuCfUfCfucaauuuucus(invAb) |
| 2 | asGfsasAfaAfuUfgAfgAfgAfaGfuCfcasc | 13 | (NAG25)s(invAb)sguggacuuCfUfCfucaauuuucus(invAb) |
| 3 | asGfsasAfaAfuUfgAfgAfgAfaGfuCfcacusu | 10 | (NAG25)sgsuggacuuCfUfCfucaauuuucus(invAb) |
| 3 | asGfsasAfaAfuUfgAfgAfgAfaGfuCfcacusu | 11 | (NAG37)s(invAb)sguggacuuCfUfCfucaauuuucus(invAb) |
| 3 | asGfsasAfaAfuUfgAfgAfgAfaGfuCfcacusu | 13 | (NAG25)s(invAb)sguggacuuCfUfCfucaauuuucus(invAb) |
| 4 | asGfsasAfaAfuUfgAfgAfgAfaGfuCfcacsc | 12 | (NAG37)s(invAb)sgguggacuuCfUfCfucaauuuucus(invAb) |
| 8 | usAfscsCfaAfuUfuAfuGfcCfuAfcAfgcsg | 16 | (NAG37)s(invAb)scgcuguagGfCfAfuaaauugguas(invAb) |
| 8 | usAfscsCfaAfuUfuAfuGfcCfuAfcAfgcsg | 17 | (NAG37)csgcuguagGfCfAfuaaauugguas(invAb) |
| 8 | usAfscsCfaAfuUfuAfuGfcCfuAfcAfgcsg | 18 | (NAG25)s(invAb)scgcuguagGfCfAfuaaauugguas(invAb) |

A=adenosine-3′-phosphate;
C=cytidine-3′-phosphate;
G=guanosine-3′-phosphate;
U=uridine-3′-phosphate
n=any 2′-OMe modified nucleotide
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Nf=any 2′-fluoro modified nucleotide
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dN=any 2′-deoxyribonucleotide
dT=2′-deoxythymidine-3′-phosphate
N_{UNA}=2′, 3′-seco nucleotide mimics (unlocked nucleobase analogs)
N_{LNA}=locked nucleotide
Nf_{ANA}=2′-F-Arabino nucleotide
NM=2′-methoxyethyl nucleotide
AM=2′-methoxyethyladenosine-3′-phosphate
AMs=2′-methoxyethyladenosine-3′-phosphorothioate
=2′-methoxyethylthymidine-3′-phosphate
TMs=2′-methoxyethylthymidine-3′-phosphorothioate
R=ribitol
(invdN)=any inverted deoxyribonucleotide (3′-3′linked nucleotide)
(invAb)=inverted (3′-3′ linked) abasic deoxyribonucleotide, see Table 2
(invAb)s=inverted (3′-3′ linked) abasic deoxyribonucleotide-5′-phosphorothioate, see Table 2
(invn)=any inverted 2′-OMe nucleotide (3′-3′linked nucleotide)
s=phosphorothioate linkage

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the RNAi component comprises a first and a second RNAi gents in a ratio of about 1:1, 2:1, 3:1, 4:1 or 5:1. In some embodiments, the two HBV RNAi agents are administered in a ratio of about 2:1.

### Interferon

Human interferons are classified into three major types based on the type of receptor through which they signal. In various embodiments, an interferon of any of Types I-III is used in combination with an RNAi component and a compound of formula (I) to treat HBV infection. All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. Type II IFNs bind to IFN-gamma receptor (IFNGR) that consists of IFNGR1 and IFNGR2 chains. The type II interferon in humans is IFN-gamma. The recently classified type III interferon group consists of three IFN-lambda molecules called IFN-lambda1, IFN-lambda2 and IFN-lambda3 (also called IL29, IL28A, and IL28B, respectively). These IFNs signal through a receptor complex consisting of IL10R2 (also called CRF2-4) and IFNLR1 (also called CRF2-12).

In one aspect, the present invention provides combination therapies in which an interferon-alpha or interferon-lambda are used in combination with an RNAi component and a compound of formula (I), and optionally a nucleoside analog. The term “interferon-alpha” or “IFN-a” and “interferon-lambda” or “IFN-λ” as used herein refers to a family of related polypeptides that inhibit viral replication and cellular proliferation and modulate immune response. Suitable interferons for purposes of the invention include, but are not limited to, pegylated IFN-a-2a, pegylated IFN-a-2b, consensus IFN, IFN-λ (e.g., IFN-λI such as IFN-X1a), or pegylated IFN-λ (e.g., pegylated IFN-λ such as pegylated IFN-Xla).

### Interferon Alpha

The term “IFN-a” includes naturally occurring IFN-a; synthetic IFN-a; derivatized IFN-a (e.g., PEGylated IFN-a, glycosylated IFN-a, and the like); and analogs of naturally occurring or synthetic IFN-a. The term “IFN-a” also encompasses consensus IFN-a. Thus, essentially any IFN-a or IFN-λ that has antiviral properties, as described for naturally occurring IFN-a, can be used in the combination therapies of the invention.

The term “IFN-a” encompasses derivatives of IFN-a that are derivatized (e.g., are chemically modified relative to the naturally occurring peptide) to alter certain properties such as serum half-life. As such, the term “IFN-a” includes IFN-a derivatized with polyethylene glycol (“PEGylated IFN-a”), and the like. PEGylated IFN-a, and methods for making same, is discussed in, e.g., U.S. Pat. Nos. 5,382,657; 5,951,974; and 5,981,709. PEGylated IFN-a encompasses conjugates of PEG and any of the above-described IFN-a molecules, including, but not limited to, PEG conjugated to interferon alpha-2a (Roferon, Hoffman La-Roche, Nutley, N.J.), interferon alpha-2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.). Thus, in some embodiments of the combination therapies of the invention, the IFN-a has been modified with one or more polyethylene glycol moieties, i.e., pegylated. Two forms of pegylated-interferon, peginterferon alfa-2a (40 kD) (Pegasys®, Genentech) and peginterferon alfa-2b (12 kD) (PegIntron®, Merck), are commercially available, which differ in terms of their pharmacokinetic, viral kinetic, tolerability profiles, and hence, dosing.

Peginterferon alfa-2a (Pegasys®) consists of interferon alfa-2a (˜20 kD) covalently linked to a 40 kD branched polyethylene glycol (PEG). The PEG moiety is linked at a single site to the interferon alfa moiety via a stable amide bond to lysine. Peginterferon alfa-2a has an approximate molecular weight of 60,000 daltons. The biologic activity of peginterferon-alfa-2a derives from its interferon alfa-2a moiety which impacts both adaptive and innate immune responses against certain viruses. This alpha interferon binds to and activates human type 1 interferon receptors on hepatocytes which activates multiple intracellular signal transduction pathways, culminating in the expression of interferon-stimulated genes that produce an array of antiviral effects, such as blocking viral protein synthesis and inducing viral RNA mutagenesis. Compared with the native interferon alfa-2a, the peginterferon alfa-2a has sustained absorption, delayed clear. Peginterferon alfa-2a is used as a fixed weekly dose. Peginterferon alfa-2a has a relatively constant absorption after injection and is distributed mostly in the blood and organs.

Peginterferon alfa-2b (PegIntron®) consists of interferon alfa-2b covalently linked to a 12 kD linear polyethylene glycol (PEG). The average molecular weight of the molecule is approximately 31,300 daltons. Peginterferon alfa-2b is predominantly composed of monopegylated species (one PEG molecule is attached to one interferon molecule), with only a small amount of dipegylated species. Fourteen different PEG attachment sites on the interferon molecule have been identified. The biologic activity of peginterferon alfa-2b derives from its interferon alfa-2b moiety, which impacts both adaptive and innate immune responses against certain viruses. This alpha interferon binds to and activates human type 1 interferon receptors on hepatocytes which activates multiple intracellular signal transduction pathways, culminating in the expression of interferon-stimulated genes that produce an array of antiviral effects, such as blocking viral protein synthesis and inducing viral RNA mutagenesis. Compared with the native interferon alfa-2b, the peginterferon alfa-2b has sustained absorption, delayed clearance, and a prolonged half-life. Peginterferon alfa-2b is used as a weekly dose based on the weight of the patient. Peginterferon alfa-2b has a rapid absorption and a wider distribution in the body.

The PEG molecule of a pegylated IFN-a polypeptide is conjugated to one or more amino acid side chains of the IFN-a polypeptide. In an embodiment, the pegylated IFN-a contains a PEG moiety on only one amino acid. In another embodiment, the pegylated IFN-a contains a PEG moiety on two or more amino acids, e.g., the IFN-a contains a PEG moiety attached to two, three, four, five, six, seven, eight, nine, ten, eleven, twelve, thirteen, or fourteen different amino acid residues. IFN-a may be coupled directly to PEG (i.e., without a linking group) through an amino group, a sulfhydryl group, a hydroxyl group, or a carboxyl group.

The term “IFN-a” also encompasses consensus IFN-a. Consensus IFN-a (also referred to as “CIFN” and “IFN-con” and “consensus interferon”) encompasses, but is not limited to, the amino acid sequences designated IFN-conl, IFN-con2 and IFN-con3 which are disclosed in U.S. Pat. Nos. 4,695,623 and 4,897,471; and consensus interferon as defined by determination of a consensus sequence of naturally occurring interferon alphas (e.g., Infergen®, Three Rivers Pharmaceuticals, Warrendale, Pa.). IFN-conl is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. In an embodiment, at least one additional therapeutic agent is CIFN.

In various embodiments of the combination therapies of the invention, fusion polypeptides comprising an IFN-a and a heterologous polypeptide are used. Suitable IFN-a fusion polypeptides include, but are not limited to, Albuferon-alpha™ (a fusion product of human albumin and IFN-a; Human Genome Sciences; see, e.g., Osbom et al., 2002, J. Pharmacol. Exp. Therap. 303:540-548). Also suitable for use in the present methods are gene-shuffled forms of IFN-a. See, e.g., Masci et al., 2003, Curr. Oncol. Rep. 5:108-113. Other suitable interferons include Multiferon (Viragen), Medusa Interferon (Flamel Technology), Locteron (Octopus), and Omega Interferon (Intarcia/Boehringer Ingelheim). Interferon lambda

The term “IFN-λ” encompasses IFN-lambda-1 (which includes IFN-lambda-1a), IFN-lambda-2, and IFN-lambda-3. These proteins are also known as interleukin-29 (IL-29), IL-28A, and IL-28B, respectively. Collectively, these 3 cytokines comprise the type III subset of IFNs. They are distinct from both type I and type II IFNs for a number of reasons, including the fact that they signal through a heterodimeric receptor complex that is different from the receptors used by type I or type II IFNs. Although type I IFNs (IFN-alpha/beta) and type III IFNs (IFN-lambda) signal via distinct receptor complexes, they activate the same intracellular signaling pathway and many of the same biological activities, including antiviral activity, in a wide variety of target cells. Interferon lambda may be administered at any therapeutically appropriate dose, including, without limitation, 80, 120 or 180 mcg QW. In some embodiments, the dose for an adult human is 120 micrograms once per week.

In some embodiments, the interferon lambda is a pegylated form of interferon lambda (e.g., pegylated interferon lambda-1 or pegylated interferon lambda-1a). In some embodiments, the interferon lambda is an interferon disclosed in U.S. Pat. No. 7,157,559 incorporated by reference herein.

While not wishing to be bound by theories, it is believed that an interferon, such as an interferon alpha or an interferon lambda, induces interferon-stimulated genes (ISGs), and/or stimulates immune response, more particularly of the Thl response, such as expression or activation of the TBET transcription factor is increased, and/or the production of IFNgamma by CD4+ T cells is increased (e.g. ratio IFNgamma/IL4 is superior to 1). The administration of interferon therefore enhances the therapeutic effects of the RNAi component and the compound of formula (I) according to the embodiments of the invention, particularly in an immune tolerant subject.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) is

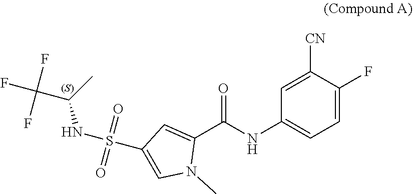

or a pharmaceutically acceptable salt thereof, and the interferon is an interferon alpha or lambda, such as pegylated interferon lambda-1, pegylated interferon lambda-1a, pegylated IFN-alfa 2a or pegylated IFN-alfa 2b

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) is

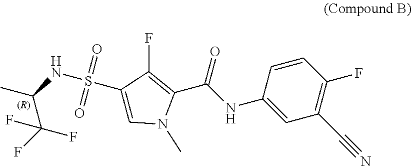

or a pharmaceutically acceptable salt thereof, and the interferon is an interferon alpha or lambda, such as pegylated interferon lambda-1, pegylated interferon lambda-1a, pegylated IFN-alfa 2a or pegylated IFN-alfa 2b.

The combinations described herein can be used in any methods or kits described below.

### Composition

Also provided is a pharmaceutical composition comprising the RNAi component, a pharmaceutical composition comprising the CAM, or a pharmaceutical composition comprising the interferon for use in combination with the other pharmaceutical compositions for treating an HBV infection or a disease or disorder associated with the HBV infection in a subject in need thereof.

In one embodiment, provided herein is a first pharmaceutical composition comprising an RNAi component for use in a combination with a second pharmaceutical composition comprising a CAM and a third pharmaceutical composition comprising an interferon for treating an HBV infection or a disease or disorder associated with the HBV infection in a subject in need thereof, wherein the RNAi component comprises:
(i) a first RNAi agent comprising: an antisense strand comprising a nucleotide sequence of any one of the following: SEQ ID NOT, SEQ ID NO:2, SEQ ID NOT, SEQ ID NO:4, SEQ ID NOT, SEQ ID NO:6, and SEQ ID NOT and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NO: 8 and SEQ ID NO:9, and a sense strand comprising a nucleotide sequence of any one of the following: SEQ ID NO:16, SEQ ID NO:17, SEQ ID NO: 18, and SEQ ID NO:19.

In one embodiment, provided herein is a second pharmaceutical composition comprising a CAM for use in a combination with a first pharmaceutical composition comprising an RNAi component and a third pharmaceutical composition comprising an interferon for treating an HBV infection or a disease or disorder associated with the HBV infection in a subject in need thereof, wherein the CAM is a compound of Formula (I),

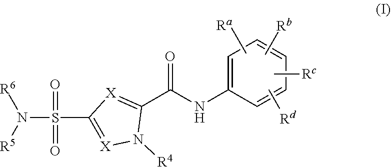

or a pharmaceutically acceptable salt thereof, wherein:
each X independently is CR⁷;
R^{a}, R^{b} and R^{c} are independently selected from the group consisting of hydrogen, fluoro, bromo, chloro, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally being substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, fluoro, chloro, bromo, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In yet another embodiment, provided herein is a third pharmaceutical composition comprising an interferon for use in a combination with a first pharmaceutical composition comprising an RNAi component and a second pharmaceutical composition comprising a CAM for treating an HBV infection or a disease or disorder associated with the HBV infection in a subject in need thereof, wherein the interferon can be, for example, an interferon alpha or lambda, preferably a pegylated interferon, more preferably a pegylated interferon alpha-2a or pegylated interferon lambda-1a.

### CAM Pharmaceutical Compositions

In some embodiments, an effective amount of the compound of Formula (I) is in the range of about 75-600 mg per dose. In some embodiments, an effective amount of the compound of Formula (I) is in the range of about 75-100 mg, about 100-150 mg, about 150-200 mg, about 200-250 mg, about 250-300 mg, about 300-400 mg, about 400-500 mg or about 500-600 mg per dose. In some embodiments, an effective amount of the compound of Formula (I) is about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, an effective amount of the compound of Formula (I) is about 100 mg, about 150 mg or about 250 mg per dose.

The compounds of the present invention, such as the compounds of Formula (I), or a pharmaceutically acceptable salt thereof, or any subgroup thereof may be formulated into various pharmaceutical forms for administration purposes. Appropriate compositions useful for the invention include all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

### RNAi Agent Pharmaceutical Compositions

In another aspect, described herein are methods for therapeutic and/or prophylactic treatment of diseases/disorders which are associated with HBV infection or inhibition of expression of one or more HBV genes comprising administering a pharmaceutical composition comprising one or more HBV RNAi agents that can be administered in a number of ways depending upon whether local or systemic treatment is desired. Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

In another aspect, methods described herein comprise one or more HBV RNAi agents, wherein the one or more HBV agents are prepared as pharmaceutical compositions or formulations. In some embodiments, pharmaceutical compositions include at least one HBV RNAi agent. These pharmaceutical compositions are particularly useful in the inhibition of the expression of the target mRNA in a target cell, a group of cells, a tissue, or an organism. The pharmaceutical compositions can be used to treat a subject having a disease or disorder that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering an HBV RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions including an HBV RNAi agent, thereby forming a pharmaceutical formulation suitable for in vivo delivery to a human.

The pharmaceutical compositions that include an HBV RNAi agent and methods disclosed herein may decrease the level of the target mRNA in a cell, group of cells, group of cells, tissue, or subject, including: administering to the subject a therapeutically effective amount of a herein described HBV RNAi agent, thereby inhibiting the expression of a target mRNA in the subject.

In some embodiments, the described pharmaceutical compositions including an HBV RNAi agent are used for treating or managing clinical presentations associated with HBV infection. In some embodiments, a therapeutically or prophylactically effective amount of one or more of pharmaceutical compositions is administered to a subject in need of such treatment, prevention or management. In some embodiments, administration of any of the disclosed HBV RNAi agents can be used to decrease the number, severity, and/or frequency of symptoms of a disease in a subject.

The described pharmaceutical compositions including an HBV RNAi agent can be used to treat at least one symptom in a subject having a disease or disorder that would benefit from reduction or inhibition in expression of HBV mRNA. In some embodiments, the subject is administered a therapeutically effective amount of one or more pharmaceutical compositions including an HBV RNAi agent thereby treating the symptom. In other embodiments, the subject is administered a prophylactically effective amount of one or more HBV RNAi agents, thereby preventing the at least one symptom.

The route of administration is the path by which an HBV RNAi agent is brought into contact with the body. In general, methods of administering drugs and nucleic acids for treatment of a mammal are well known in the art and can be applied to administration of the compositions described herein. The HBV RNAi agents disclosed herein can be administered via any suitable route in a preparation appropriately tailored to the particular route. Thus, herein described pharmaceutical compositions can be administered by injection, for example, intravenously, intramuscularly, intracutaneously, subcutaneously, intraarticularly, or intraperitoneally. In some embodiments, there herein described pharmaceutical compositions via subcutaneous injection.

The pharmaceutical compositions including an HBV RNAi agent described herein can be delivered to a cell, group of cells, tumor, tissue, or subject using oligonucleotide delivery technologies known in the art. In general, any suitable method recognized in the art for delivering a nucleic acid molecule (in vitro or in vivo) can be adapted for use with a herein described compositions. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the herein described pharmaceutical compositions may comprise one or more pharmaceutically acceptable excipients. In some embodiments, the pharmaceutical compositions described herein can be formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical ingredient (API, therapeutic product, e.g., HBV RNAi agent) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-fdtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, poly orthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The HBV RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic or preventive result.

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active compound will be in the range of 25-400 mg per 1-18 weeks or 1-6 months. In some embodiments, an effective amount of an active compound will be in the range of 50-125 mg per 4 weeks or per one month. In some embodiments, an effective amount of an active ingredient will be in the range of from about 0.5 to about 3 mg/kg of body weight per dose. In some embodiments, an effective amount of an active ingredient will be in the range of from about 25-400 mg per dose. In some embodiments, an effective amount of an active ingredient will be in the range of from about 50-125 mg per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

In some embodiments, an effective amount of the RNAi component is in the range of about 25-600 mg per dose. In some embodiments, an effective amount of the RNAi component is in the range of about 25-50 mg, about 50-75 mg, about 75-100 mg, about 100-150 mg, about 150-200 mg, about 200-250 mg, about 250-300 mg, about 300-400 mg, about 400-500 mg or about 500-600 mg per dose. In some embodiments, an effective amount of the RNAi component is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, an effective amount of the RNAi component is about 25 mg, about 35 mg, about 40 mg, about 50 mg, about 100 mg or about 200 mg per dose.

The one or more (e.g., at least two) HBV RNAi agents described herein can be formulated into one single composition or separate individual compositions. In some embodiments, the HBV RNAi agents in separate individual compositions can be formulated with the same or different excipients and carriers. In some embodiments, the HBV RNAi agents in separate individual compositions agents can be administered through same or different administration routes. In some embodiments, the HBV RNAi agents are administered subcutaneously.

For treatment of disease or for formation of a medicament or composition for treatment of a disease, the pharmaceutical compositions described herein including an HBV RNAi agent can be combined with an excipient or with a second therapeutic agent or treatment including, but not limited to: a second or other RNAi agent, a small molecule drug, an antibody, an antibody fragment, and/or a vaccine.

The described HBV RNAi agents, when added to pharmaceutically acceptable excipients or adjuvants, can be packaged into kits, containers, packs, or dispensers. The pharmaceutical compositions described herein may be packaged in pre-fdled syringes or vials.

In some embodiments, the composition comprises an effective amount of an RNAi component in the range of about 25-600 mg and an effective amount of a compound of Formula (I) in the range of about 75-600 mg per dose. In some embodiments, the composition comprises an effective amount of an RNAi component in the range of about 25-300 mg and an effective amount of a compound of Formula (I) in the range of about 75-300 mg per dose. In some embodiments, the composition comprises an effective amount of an RNAi of about 25 mg, about 35 mg, about 40 mg, about 50 mg, about 100 mg or about 200 mg and an effective amount of a compound of Formula (I) of about 100 mg, about 150 mg or about 250 mg per dose.

### Interferon Pharmaceutical Composition

Any suitable pharmaceutical compositions of interferon, including those known in the art and/or described herein, can be used in the combination therapy of the application. The composition can be administered in the range of about 25-500 mcg per dose, more particularly 25-360 mcg per dose.

### Kit

Provided herein is a kit comprising an effective amount of an RNAi component, an effective amount of a compound of Formula (I) or a pharmaceutically acceptable salt thereof, and an effective amount of an interferon, wherein the RNAi component, the compound of Formula (I) and the interferon are as described herein.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In some embodiments, the kit comprises one or more doses of the compound of Formula (I) in the range of about 75-600 mg per dose. In some embodiments, the kit comprises one or more doses of the compound of Formula (I) in the range of about 75-100 mg, about 100-150 mg, about 150-200 mg, about 200-250 mg, about 250-300 mg, about 300-400 mg, about 400-500 mg or about 500-600 mg per dose. In some embodiments, the kit comprises one or more doses of the compound of Formula (I) of about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments the kit comprises one or more doses of the compound of Formula (I) of about 100 mg, about 150 mg or about 250 mg per dose.

In some embodiments, the kit comprises one or more doses of the RNAi component in the range of about 25-600 mg per dose. In some embodiments, the kit comprises one or more doses of the RNAi component in the range of about 25-50 mg, about 50-75 mg, about 75-100 mg, about 100-150 mg, about 150-200 mg, about 200-250 mg, about 250-300 mg, about 300-400 mg, about 400-500 mg or about 500-600 mg per dose. In some embodiments, the kit comprises one or more doses of the RNAi component of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, the kit comprises one or more doses of the RNAi component of about 25 mg, about 35 mg, about 40 mg, about 50 mg, about 100 mg or about 200 mg per dose.

In some embodiments, the kit comprises one or more doses of the interferon in the range of about 10-25 meg, about 25-100 meg, about 50-100 meg, about 100-150 meg, about 150-200 meg, about 200-250 meg, about 250-300 meg, about 300-400 meg, or about 400-500 meg, per dose. In some embodiments, the kit comprises one or more doses of pegylated IFN lambda-1a, pegylated interferon alfa-2a or pegylated IFN lambda-1a, of about 75 meg, about 80 meg, about 85 meg, about 90 meg, about 95 meg, about 100 meg, about 125 meg, about 150 meg, about 175 meg, about 200 meg, about 250 meg, about 300 mcg per dose.

In some embodiments, the kit contains an RNAi component useful for the invention, such as those described herein, for once monthly administration to a subject in a dose of about 40-250 mg, more particularly 40-200 mg, more particularly 200 mg; a compound of Formula (I) or a pharmaceutically acceptable salt thereof, such as those described herein, for daily administration to a subject in a dose of about 100-500 mg, particularly of 200-300 mg, more particularly of 250 mg; and an interferon, such as those described herein for intravenous or subcutaneous injection in a dose of about 25-500 meg, preferably 80-300 meg, per week, preferably once per week, more particularly of 100-200 meg per week, more particularly 180 mcg per week.

In some embodiments, the kit further comprises instructions for using the RNAi component, the compound of Formula (I) and the interferon contained therein for administration to a subject infected by HBV, in particular, a subject having a chronic HBV infection, more particularly the subject is infected by HBV but is immune tolerant.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more. In some embodiments, one cycle of treatment is about 1-24 months, about 1-3 months, about 3-6 months, about 6-9 months, about 9-12 months, about 12-18 months, about 18-21 months or about 21-24 months. In some embodiments, one cycle of treatment is about 1 month, about 2 months, about 3 months, about 4 months, about 5 months, about 6 months, about 7 months, about 8 months, about 9 months, about 10 months, about 11 months, about 12 months, about 15 months, about 18 months, about 21 months or about 24 months.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### Method

Also provided herein is a method for inhibiting the expression of a Hepatitis B Virus gene in a subject in need thereof, wherein the method comprises administering to the subject an effective amount of an RNAi component, a capsid assembly modulator of formula (I) or a pharmaceutically acceptable salt thereof and an interferon, such as those described herein. Also provided herein is a method for treating a disease or disorder associated with an HBV infection in a subject, wherein the method comprises administering to the subject an effective amount of an RNAi component, a capsid assembly modulator of formula (I) or a pharmaceutically acceptable salt thereof and an interferon, such as those described herein. Also provided herein is a method for treating a disease or disorder associated with an HBV infection in a subject receiving a capsid assembly modulator therapy, wherein the method comprises administering to the subject an effective amount of an RNAi component and an interferon, such as those describe herein. Also provided herein is a method for treating a disease or disorder associated with an HBV infection in a subject receiving an interferon therapy, wherein the method comprises administering to the subject an effective amount of an RNAi component and a capsid assembly modulator of formula (I) or a pharmaceutically acceptable salt thereof. Also provided herein is a method for treating a disease or disorder associated with an HBV infection in a subject receiving a nucleoside analog or nucleotide analog therapy, wherein the method comprises administering to the subject an effective amount of an RNAi component, a capsid assembly modulator of formula (I) or a pharmaceutically acceptable salt thereof and an interferon, such as those describe herein. Also provided herein is a method for treating a hepatitis B virus infection comprising contacting a cell infected with the Hepatitis B Virus infection with an effective amount of an RNAi component, a capsid assembly modulator of formula (I) or a pharmaceutically acceptable salt thereof and an interferon, such as those described herein. Also provided herein is a combination for use in treating an HBV infection or disease or disorder associated with an HBV infection, wherein the combination comprises, an RNAi component, a capsid assembly modulator of Formula (I) or a pharmaceutically acceptable salt thereof, and an interferon, such as those described herein. In some embodiments, the combination is for simultaneous, separate, or sequential use.

Also provided herein is a combination, wherein the combination comprises: (a) the RNAi component, (b) a compound of Formula (I) or a pharmaceutically acceptable salt thereof, and (c) interferon, for use in the treatment of HBV infection (more particularly chronic HBV infection) in a subject in need thereof, more particularly for simultaneous, sequential or separate use in the treatment of HBV infection (more particularly chronic HBV infection) in a subject in need thereof. Also provided herein is a RNAi component for use in the treatment of CHB or a disease or disorder associated with an HBV infection in a subject, wherein the treatment comprises: (a) the administration of the RNAi component, (b) the administration of a compound of Formula (I) or a pharmaceutically acceptable salt thereof, and (c) the administration of interferon. Also provided herein is a compound of Formula (I) or a pharmaceutically acceptable salt thereof for use in the treatment of CHB or a disease or disorder associated with an infection caused by Hepatitis B Virus in a subject, wherein the treatment comprises: (a) the administration of the compound of Formula (I) or a pharmaceutically acceptable salt thereof, (b) the administration of a RNAi component, and (c) the administration of interferon. Also provided herein is interferon for use in the treatment of CHB or a disease or disorder associated with an HBV infection in a subject, wherein the treatment comprises: (a) the administration of interferon, (b) the administration of a RNAi component, and (c) the administration of a compound of Formula (I) or a pharmaceutically acceptable salt thereof. The RNAi component, the compound of Formula (I) or a pharmaceutically acceptable salt thereof, and interferon are advantageously used in therapeutically effective amounts in a combination therapy for the treatment of HBV infection (more particularly chronic HBV infection).

A “disease or disorder associated with an HBV infection”, also referred to herein as a “disease or disorder associated with an infection caused by Hepatitis B Virus,” can include any disease or disorder that is associated with or caused by an HBV infection. In certain embodiment, the disease or disorder associated with the HBV infection comprises one or more selected from the group consisting of jaundice, fulminant hepatitis, liver injury, liver failure, end-stage liver disease, cirrhosis, and liver cancer (such as hepatocellular carcinoma). In some embodiments, a subject under a treatment of the application is infected with an HBV, more particularly the subject has a chronic HBV infection, even more particularly, the subject has a chronic HBV infection and another infection (more particularly chronic infection) with at least one of: hepatitis D virus (HDV); hepatitis C virus (HCV); or human immunodeficiency virus (HIV).

Also provided herein is a combination, wherein the combination comprises: (a) the RNAi component, (b) a compound of Formula (I) or a pharmaceutically acceptable salt thereof, and (c) interferon, for use in the treatment of HDV infection (more particularly chronic HDV infection) in a subject in need thereof, more particularly for simultaneous, sequential or separate use in the treatment of HDV infection (more particularly chronic HDV infection) in a subject in need thereof. Also provided herein is a RNAi component for use in the treatment of HDV infection, more particularly chronic HDV infection, in a subject in need thereof, wherein the treatment comprises: (a) the administration of the RNAi component, (b) the administration of a compound of Formula (I) or a pharmaceutically acceptable salt thereof, and (c) the administration of interferon. Also provided herein is a compound of Formula (I) or a pharmaceutically acceptable salt thereof for use in the treatment of HDV infection, more particularly chronic HDV infection, in a subject in need thereof, wherein the treatment comprises: (a) the administration of the compound of Formula (I) or a pharmaceutically acceptable salt thereof, (b) the administration of a RNAi component, and (c) the administration of interferon. Also provided herein is interferon for use in the treatment of HDV infection, more particularly chronic HDV infection, in a subject in need thereof, wherein the treatment comprises: (a) the administration of interferon, (b) the administration of a RNAi component, and (c) the administration of a compound of Formula (I) or a pharmaceutically acceptable salt thereof. The RNAi component, the compound of Formula (I) or a pharmaceutically acceptable salt thereof, and interferon are advantageously used in therapeutically effective amounts in a combination therapy for the treatment of HDV infection (more particularly chronic HDV infection).

In some embodiments, the RNAi component comprises: (i) a first RNAi agent comprising: an antisense strand comprising a nucleotide sequence of any one of the following: SEQ ID NOT, SEQ ID NO:2, SEQ ID NOT, SEQ ID NO:4, SEQ ID NOT, SEQ ID NO:6, and SEQ ID NO:7, and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and (ii) a second RNAi agent comprising: an antisense strand comprising a nucleotide sequence of any one of the following: SEQ ID NOT and SEQ ID NOV, and a sense strand comprising a nucleotide sequence of any one of the following: SEQ ID NO: 16, SEQ ID NO: 17, SEQ ID NO: 18, and SEQ ID NO: 19.

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the two HBV RNAi agents are administered in a ratio of about 1:1, 2:1, 3:1, 4:1 or 5:1. In some embodiments, the two HBV RNAi agents are administered in a ratio of about 2:1.

In some embodiments, the two HBV RNAi agents are administered in a combined amount of about 25-75 mg per dose administration and in the ratio of about 2:1, about 3:1, about 1:1, about 4:1, about 5:1 or about 1:2. In some embodiments, the two HBV RNAi agents are administered in a combined amount of about 50-125 mg per dose administration and in the ratio of about 2:1, about 3:1, about 1:1, about 4:1, about 5:1 or about 1:2. In some embodiments, the two HBV RNAi agents are administered in a combined amount of about 75-150 mg per dose administration and in the ratio of about 2:1, about 3:1, about 1:1, about 4:1, about 5:1 or about 1:2. In some embodiments, the two HBV RNAi agents are administered in a combined amount of about 100-200 mg per dose administration and in the ratio of about 2:1, about 3:1, about 1:1, about 4:1, about 5:1 or about 1:2. In some embodiments, the two HBV RNAi agents are administered in a combined amount of about 150-250 mg per dose administration and in the ratio of about 2:1, about 3:1, about 1:1, about 4:1, about 5:1 or about 1:2. In some embodiments, the two HBV RNAi agents are administered in a combined amount of about 200-300 mg per dose administration and in the ratio of about 2:1, about 3:1, about 1:1, about 4:1, about 5:1 or about 1:2. In some embodiments, the two HBV RNAi agents are administered in a combined amount of about 300-400 mg per dose administration and in the ratio of about 2:1, about 3:1, about 1:1, about 4:1, about 5:1 or about 1:2. In some embodiments, the two HBV RNAi agents are administered in a combined amount of about 50-100 mg per dose administration and in the ratio of about 2:1, about 3:1, about 1:1, about 4:1, about 5:1 or about 1:2. In some embodiments, the two HBV RNAi agents are administered in a combined amount of about 25-400 mg per dose administration and in the ratio of about 2:1. In some embodiments, the two HBV RNAi agents are administered in a combined amount of about 25-75 mg per dose administration and in the ratio of about 2:1. In some embodiments, the two HBV RNAi agents are administered in a combined amount of about 50-125 mg per dose administration and in the ratio of about 2:1. In some embodiments, the two HBV RNAi agents are administered in a combined amount of about 75-150 mg per dose administration and in the ratio of about 2:1. In some embodiments, the two HBV RNAi agents are administered in a combined amount of about 100-200 mg per dose administration and in the ratio of about 2:1. In some embodiments, the two HBV RNAi agents are administered in a combined amount of about 125-225 mg per dose administration and in the ratio of about 2:1. In some embodiments, the two HBV RNAi agents are administered in a combined amount of about 150-250 mg per dose administration and in the ratio of about 2:1. In some embodiments, the two HBV RNAi agents are administered in a combined amount of about 200-300 mg per dose administration and in the ratio of about 2:1. In some embodiments, the two HBV RNAi agents are administered in a combined amount of about 300-400 mg per dose administration and in the ratio of about 2:1. In some embodiments, the two HBV RNAi agents are administered in a combined amount of about 100 mg per dose administration and in the ratio of about 2:1. In some embodiments, the two HBV RNAi agents are administered in a combined amount of about 25 mg per dose administration and in the ratio of about 2:1. In some embodiments, the two HBV RNAi agents are administered in a combined amount of about 35 mg per dose administration and in the ratio of about 2:1. In some embodiments, the two HBV RNAi agents are administered in a combined amount of about 40 mg per dose administration and in the ratio of about 2:1. In some embodiments, the two HBV RNAi agents are administered in a combined amount of about 50 mg per dose administration and in the ratio of about 2:1. In some embodiments, the two HBV RNAi agents are administered in a combined amount of about 75 mg per dose administration and in the ratio of about 2:1. In some embodiments, the two HBV RNAi agents are administered in a combined amount of about 200 mg per dose administration and in the ratio of about 2:1.

In some embodiments, the first RNAi agent is administered in an amount of about 3-650 mg per dose administration, and the second RNAi agent is administered in an amount of about 2-325 mg per dose administration. In some embodiments, the first RNAi agent is administered in an amount of about 15-150 mg per dose administration, and the second RNAi agent is administered in an amount of about 5-75 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the first RNAi agent is administered in an amount of about 15-75 mg per dose administration. In some embodiments, the second RNAi agent is administered in an amount of about 20-125 mg per dose administration. In some embodiments, the second RNAi agent is administered in an amount of about 25-50 mg per dose administration. In some embodiments, the second RNAi agent is administered in an amount of about 5-40 mg per dose administration. In some embodiments, the first RNAi agent is administered in an amount of about 17 mg per dose administration, and the second RNAi agent is administered in an amount of about 8 mg per dose administration. In some embodiments, the first RNAi agent is administered in an amount of about 23 mg per dose administration, and the second RNAi agent is administered in an amount of about 12 mg per dose administration. In some embodiments, the first RNAi agent is administered in an amount of about 27 mg per dose administration, and the second RNAi agent is administered in an amount of about 13 mg per dose administration. In some embodiments, the first RNAi agent is administered in an amount of about 33 mg per dose administration, and the second RNAi agent is administered in an amount of about 17 mg per dose administration. In some embodiments, the first RNAi agent is administered in an amount of about 67 mg per dose administration, and the second RNAi agent is administered in an amount of about 33 mg per dose administration.

In some embodiments, two RNAi agents are administered at a combined dose of 25-400 mg per dose administration. In an embodiment, two RNAi agents are administered at a combined dose of 25-400 mg, and the first RNAi agent is administered with the second RNAi agent at a ratio of 1:1. In an embodiment, the dose of each of the first and second RNAi agents is in an amount of about 12 mg for a combined dose of about 25 mg. In an embodiment, the dose of each of the first and second RNAi agents is in an amount of about 17 mg for a combined dose of about 35 mg. In an embodiment, the dose of each of the first and second RNAi agents is in an amount of about 20 mg for a combined dose of about 40 mg. In an embodiment, the dose of each of the first and second RNAi agents is in an amount of about 25 mg for a combined dose of about 50 mg. In an embodiment, the dose of each of the first and second RNAi agents is in an amount of about 50 mg for a combined dose of about 100 mg. In an embodiment, the dose of each of the first and second RNAi agents is in an amount of about 100 mg for a combined dose of about 200 mg. In an embodiment, the dose of each of the first and second RNAi agents is in an amount of about 150 mg for a combined dose of about 300 mg. In an embodiment, the dose of each of the first and second RNAi agents is in an amount of about 200 mg for a combined dose of about 400 mg.

In an embodiment, two RNAi agents are administered at a combined dose of 25-400 mg per dose, and the first RNAi agent is administered with the second RNAi agent at a ratio of 2:1. In an embodiment, the dose of the first RNAi agent is in an amount of about 16 mg, and the dose of the second RNAi agent is in an amount of about 8 mg for a combined dose of about 25 mg. In an embodiment, the dose of the first RNAi agent is in an amount of about 24 mg, and the dose of the second RNAi agent is in an amount of about 12 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the first RNAi agent is in an amount of about 65 mg, and the dose of the second RNAi agent is in an amount of about 35 mg for a combined dose of about 100 mg. In an embodiment, the dose of the first RNAi agent is in an amount of about 133 mg, and the dose of the second RNAi agent is in an amount of about 67 mg for a combined dose of about 200 mg. In an embodiment, the dose of the first RNAi agent is in an amount of about 200 mg, and the dose of the second RNAi agent is in an amount of about 100 mg for a combined dose of about 300 mg. In an embodiment, the dose of the first RNAi agent is in an amount of about 270 mg, and the dose of the second RNAi agent is in an amount of about 135 mg for a combined dose of about 400 mg.

In an embodiment, two RNAi agents are administered at a combined dose of 25-400 mg per dose, the first RNAi agent is administered with the second RNAi agent at a ratio of 3:1. In an embodiment, the dose of the first RNAi agent is in an amount of about 18 mg, and the dose of the second RNAi agent is in an amount of about 6 mg for a combined dose of about 25 mg. In an embodiment, the dose of the first RNAi agent is in an amount of about 27 mg, and the dose of the second RNAi agent is in an amount of about 9 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the first RNAi agent is in an amount of about 75 mg, and the dose of the second RNAi agent is in an amount of about 25 mg for a combined dose of about 100 mg. In an embodiment, the dose of the first RNAi agent is in an amount of about 150 mg, and the dose of the second RNAi agent is in an amount of about 50 mg for a combined dose of about 200 mg. In an embodiment, the dose of the first RNAi agent is in an amount of about 225 mg, and the dose of the second RNAi agent is in an amount of about 75 mg for a combined dose of about 300 mg. In an embodiment, the dose of the first RNAi agent is in an amount of about 300 mg, and the dose of the second RNAi agent is in an amount of about 100 mg for a combined dose of about 400 mg.

In some embodiments, the two HBV RNAi agents are administered in a combined amount of about 1-10 mg/kg per dose administration. In some embodiments, the two HBV RNAi agents are administered in a combined amount of about 1-5 mg/kg per dose administration. In some embodiments, the two HBV RNAi agents are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the first RNAi agent is administered in an amount of about 0.6-7 mg/kg per dose administration, and the second RNAi agent is administered in an amount of about 0.3-5 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the two RNAi agents are administered in about 1-18 week intervals. In some embodiments, the two RNAi agents are administered in about 1-week intervals, about 2-week intervals, about 3-week intervals, about 4-week intervals, about 5-week intervals, about 6-week intervals, about 7-week intervals, about 8-week intervals, about 9-week intervals, about 10-week intervals, about 11-week intervals, about 12-week intervals, about 13-week intervals, about 14-week intervals, about 15-week intervals, about 16-week intervals, about 17-week intervals, or about 18-week intervals. In some embodiments, the two RNAi agents are administered in about 1-6 month intervals. In some embodiments, the two RNAi agents are administered in about 1-month intervals, about 2-month intervals, about 3-month intervals, about 4-month intervals, about 5-month intervals, or about 6-month intervals. In some embodiments, the two RNAi agents are administered in about 4-week intervals or 1-month intervals. In some embodiments, the two RNAi agents are administered once per month.

In some embodiments, disclosed herein are methods for inhibiting the expression of a Hepatitis B Virus gene in a subject in need thereof comprising administering to a subject in need thereof an effective amount of a first RNAi agent comprising SEQ ID NO: 2 and SEQ ID NO: 11 and an effective amount of a second RNAi agent comprising SEQ ID NO: 16 and SEQ ID NO: 8. In some embodiments, disclosed herein are methods for treating a disease or disorder associated with an infection caused by Hepatitis B Virus in a subject comprising administering to a subject in need thereof an effective amount of a first RNAi agent comprising SEQ ID NO: 2 and SEQ ID NO: 11 and an effective amount of a second RNAi agent comprising SEQ ID NO: 16 and SEQ ID NO: 8. In some embodiments, the ratio of the first RNAi agent to the second RNAi agent administered to a subject in need thereof is about 2:1. In some embodiments, the ratio of the first RNAi agent to the second RNAi agent administered to a subject in need thereof is about 3:1. In some embodiments, the ratio of the first RNAi agent to the second RNAi agent administered to a subject in need thereof is about 1:1. In some embodiments, the ratio of the first RNAi agent to the second RNAi agent administered to a subject in need thereof is about 4:1. In some embodiments, the ratio of the first RNAi agent to the second RNAi agent administered to a subject in need thereof is about 5:1. In some embodiments, the ratio of the first RNAi agent to the second RNAi agent administered to a subject in need thereof is about 1:2.

In some embodiments, the first RNAi agent and the second RNAi agent are administered in a combined amount of about 25-400 mg per dose administration. In some embodiments, the first RNAi agent and the second RNAi agent are administered in a combined amount of about 25-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 25-200 mg, or 200-400 mg per dose administration. In some embodiments, the first RNAi agent to the second RNAi agent are administered in a combined amount of about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg per dose administration. In some embodiments, the first RNAi agent and the second RNAi agent are administered in a combined amount of about 50 mg, about 75 mg, about 100 mg, or about 125 mg per dose administration. In some embodiments, the first RNAi agent and the second RNAi agent are administered in a combined amount of about 25 mg, about 35 mg, about 40 mg, or about 200 mg per dose administration.

In some embodiments, the first RNAi agent and the second RNAi agent are administered in about 1-18 week intervals. In some embodiments, the first RNAi agent and the second RNAi agent are administered in about 1-week intervals, about 2-week intervals, about 3-week intervals, about 4-week intervals, about 5-week intervals, about 6-week intervals, about 7-week intervals, about 8-week intervals, about 9-week intervals, about 10-week intervals, about 11-week intervals, about 12-week intervals, about 13-week intervals, about 14-week intervals, about 15-week intervals, about 16-week intervals, about 17-week intervals, or about 18-week intervals. In some embodiments, the first RNAi agent and the second RNAi agent are administered in about 1-6 month intervals. In some embodiments, the first RNAi agent and the second RNAi agent are administered in about 1-month intervals, about 2-month intervals, about 3-month intervals, about 4-month intervals, about 5-month intervals, or about 6-month intervals. In some embodiments, the first RNAi agent and the second RNAi agent are administered in about 4-week intervals or 1-month intervals.

In some embodiments, the first RNAi agent and the second RNAi agent are administered for a duration of about 1-12 months. In some embodiments, the first RNAi agent and the second RNAi agent are administered for a duration of at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 12 months. In some embodiments, the first RNAi agent and the second RNAi agent are administered for a duration of about 1-18 weeks. In some embodiments, the first RNAi agent and the second RNAi agent are administered for a duration of at least about 1 week, at least about 5 weeks, at least about 10 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, or at least about 90 weeks. In some embodiments, capsid assembly modulator or a pharmaceutically acceptable salt is administered for a duration of about 24 weeks or 48 weeks.

In some embodiments, the first RNAi agent and the second RNAi agent are administered in a combined amount of about 25-75 mg per dose administration and in the ratio of about 2:1, about 3:1, about 1:1, about 4:1, about 5:1 or about 1:2. In some embodiments, the first RNAi agent and the second RNAi agent are administered in a combined amount of about 40-100 mg per dose administration and in the ratio of about 2:1, about 3:1, about 1:1, about 4:1, about 5:1 or about 1:2. In some embodiments, the first RNAi agent and the second RNAi agent are administered in a combined amount of about 50-125 mg per dose administration and in the ratio of about 2:1, about 3:1, about 1:1, about 4:1, about 5:1 or about 1:2. In some embodiments, the first RNAi agent and the second RNAi agent are administered in a combined amount of about 75-150 mg per dose administration and in the ratio of about 2:1, about 3:1, about 1:1, about 4:1, about 5:1 or about 1:2. In some embodiments, the first RNAi agent and the second RNAi agent are administered in a combined amount of about 100-200 mg per dose administration and in the ratio of about 2:1, about 3:1, about 1:1, about 4:1, about 5:1 or about 1:2. In some embodiments, the first RNAi agent and the second RNAi agent are administered in a combined amount of about 150-250 mg per dose administration and in the ratio of about 2:1, about 3:1, about 1:1, about 4:1, about 5:1 or about 1:2. In some embodiments, the first RNAi agent and the second RNAi agent are administered in a combined amount of about 200-300 mg per dose administration and in the ratio of about 2:1, about 3:1, about 1:1, about 4:1, about 5:1 or about 1:2. In some embodiments, the first RNAi agent and the second RNAi agent are administered in a combined amount of about 300-400 mg per dose administration and in the ratio of about 2:1, about 3:1, about 1:1, about 4:1, about 5:1 or about 1:2. In some embodiments, the first RNAi agent and the second RNAi agent are administered in a combined amount of about 50-100 mg per dose administration and in the ratio of about 2:1, about 3:1, about 1:1, about 4:1, about 5:1 or about 1:2. In some embodiments, the first RNAi agent and the second RNAi agent are administered in a combined amount of about 35-40 mg per dose administration and in the ratio of about 2:1, about 3:1, about 1:1, about 4:1, about 5:1 or about 1:2. In some embodiments, the first RNAi agent and the second RNAi agent are administered in a combined amount of about 25-400 mg per dose administration and in the ratio of about 2:1. In some embodiments, the first RNAi agent and the second RNAi agent are administered in a combined amount of about 25-75 mg per dose administration and in the ratio of about 2:1. In some embodiments, the first RNAi agent and the second RNAi agent are administered in a combined amount of about 35-40 mg per dose administration and in the ratio of about 2:1. In some embodiments, the first RNAi agent and the second RNAi agent are administered in a combined amount of about 50-125 mg per dose administration and in the ratio of about 2:1. In some embodiments, the first RNAi agent and the second RNAi agent are administered in a combined amount of about 75-150 mg per dose administration and in the ratio of about 2:1. In some embodiments, the first RNAi agent and the second RNAi agent are administered in a combined amount of about 100-200 mg per dose administration and in the ratio of about 2:1. In some embodiments, the first RNAi agent and the second RNAi agent are administered in a combined amount of about 125-225 mg per dose administration and in the ratio of about 2:1. In some embodiments, the first RNAi agent and the second RNAi agent are administered in a combined amount of about 150-250 mg per dose administration and in the ratio of about 2:1. In some embodiments, the first RNAi agent and the second RNAi agent are administered in a combined amount of about 200-300 mg per dose administration and in the ratio of about 2:1. In some embodiments, the first RNAi agent and the second RNAi agent are administered in a combined amount of about 300-400 mg per dose administration and in the ratio of about 2:1. In some embodiments, the first RNAi agent and the second RNAi agent are administered in a combined amount of about 100 mg per dose administration and in the ratio of about 2:1. In some embodiments, the first RNAi agent and the second RNAi agent are administered in a combined amount of about 25 mg per dose administration and in the ratio of about 2:1. In some embodiments, the first RNAi agent and the second RNAi agent are administered in a combined amount of about 35 mg per dose administration and in the ratio of about 2:1. In some embodiments, the first RNAi agent and the second RNAi agent are administered in a combined amount of about 40 mg per dose administration and in the ratio of about 2:1. In some embodiments, the first RNAi agent and the second RNAi agent are administered in a combined amount of about 50 mg per dose administration and in the ratio of about 2:1. In some embodiments, the first RNAi agent and the second RNAi agent are administered in a combined amount of about 200 mg per dose administration and in the ratio of about 2:1.

In some embodiments, the first RNAi agent is administered in an amount of about 3-650 mg, and the second RNAi agent is administered in an amount of about 2-325 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the second RNAi agent is administered in an amount of about 20-125 mg per dose administration. In some embodiments, the second RNAi agent is administered in an amount of about 25-50 mg per dose administration.

In some embodiments, the first RNAi agent and the second RNAi agent are administered in a combined amount of about 1-10 mg/kg per dose administration. In some embodiments, the first RNAi agent and the second RNAi agent are administered in a combined amount of about 1-5 mg/kg per dose administration. In some embodiments, the first RNAi agent and the second RNAi agent are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the second RNAi agent is administered in an amount of about 0.3-5 mg/kg per dose administration, and the first RNAi agent is administered in an amount of about 0.6-7 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the first RNAi agent and the second RNAi agent are administered at a combined dose of 25-400 mg per dose administration. In an embodiment, the first RNAi agent and the second RNAi agent are administered at a combined dose of 25-400 mg, and the first RNAi agent is administered with the second RNAi agent at a ratio of 1:1. In an embodiment, the dose of the first RNAi agent is administered with the second RNAi agent is in an amount of about 12 mg for a combined dose of about 25 mg. In an embodiment, the dose of each of the first RNAi agent and the second RNAi agent is in an amount of about 17 mg for a combined dose of about 35 mg. In an embodiment, the dose of each of the first RNAi agent and the second RNAi agent is in an amount of about 20 mg for a combined dose of about 40 mg. In an embodiment, the dose of each of the first RNAi agent and the second RNAi agent is in an amount of about 25 mg for a combined dose of about 50 mg. In an embodiment, the dose of each of the first RNAi agent and the second RNAi agent is in an amount of about 50 mg for a combined dose of about 100 mg. In an embodiment, the dose of each of the first RNAi agent and the second RNAi agent is in an amount of about 100 mg for a combined dose of about 200 mg. In an embodiment, the dose of each of the first RNAi agent and the second RNAi agent is in an amount of about 150 mg for a combined dose of about 300 mg. In an embodiment, the dose of each of the first RNAi agent and the second RNAi agent is in an amount of about 200 mg for a combined dose of about 400 mg.

In an embodiment, the first RNAi agent and the second RNAi agent are administered at a combined dose of 25-400 mg per dose, and the second RNAi agent is administered with the first RNAi agent at a ratio of 1:2. In an embodiment, the dose of the first RNAi agent is in an amount of about 16 mg, and the dose of the second RNAi agent is in an amount of about 8 mg for a combined dose of about 25 mg. In an embodiment, the dose of the second RNAi agent is in an amount of about 12 mg, and the dose of the first RNAi agent is in an amount of about 24 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the second RNAi agent is in an amount of about 35 mg, and the dose of the first RNAi agent is in an amount of about 65 mg for a combined dose of about 100 mg. In an embodiment, the dose of the second RNAi agent is in an amount of about 67 mg, and the dose of the first RNAi agent is in an amount of about 133 mg for a combined dose of about 200 mg. In an embodiment, the dose of the second RNAi agent is in an amount of about 100 mg, and the dose of the first RNAi agent is in an amount of about 200 mg for a combined dose of about 300 mg. In an embodiment, the dose of the second RNAi agent is in an amount of about 135 mg, and the dose of the first RNAi agent is in an amount of about 270 mg for a combined dose of about 400 mg.

In an embodiment, the first RNAi agent and the second RNAi agent are administered at a combined dose of 25-400 mg per dose, the second RNAi agent is administered with the first RNAi agent at a ratio of 1:3. In an embodiment, the dose of the first RNAi agent is in an amount of about 18 mg, and the dose of the second RNAi agent is in an amount of about 6 mg for a combined dose of about 25 mg. In an embodiment, the dose of the second RNAi agent is in an amount of about 9 mg, and the dose of the first RNAi agent is in an amount of about 27 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the second RNAi agent is in an amount of about 25 mg, and the dose of the first RNAi agent is in an amount of about 75 mg for a combined dose of about 100 mg. In an embodiment, the dose of the second RNAi agent is in an amount of about 50 mg, and the dose of the first RNAi agent is in an amount of about 150 mg for a combined dose of about 200 mg. In an embodiment, the dose of the second RNAi agent is in an amount of about 75 mg, and the dose of the first RNAi agent is in an amount of about 225 mg for a combined dose of about 300 mg. In an embodiment, the dose of the second RNAi agent is in an amount of about 100 mg, and the dose of the first RNAi agent is in an amount of about 300 mg for a combined dose of about 400 mg.

In some embodiments, about 1 mg/kg (mpk) of the first RNAi agent and about 1 mg/kg of the second RNAi agent are administered to a subject in need thereof. In some embodiments, about 1.5 mg/kg of the first RNAi agent and about 1.5 mg/kg of the second RNAi agent are administered to a subject in need thereof. In some embodiments, about 2.0 mg/kg of the first RNAi agent and about 1.0 mg/kg of the second RNAi agent are administered to a subject in need thereof. In some embodiments, about 3.0 mg/kg of the first RNAi agent and about 1.0 mg/kg of the second RNAi agent are administered to a subject in need thereof. In some embodiments, about 3.2 mg/kg of the first RNAi agent and about 0.8 mg/kg of the second RNAi agent are administered to a subject in need thereof. In some embodiments, about 2.7 mg/kg of the first RNAi agent and about 1.3 mg/kg of the second RNAi agent are administered to a subject in need thereof. In some embodiments, about 4.0 mg/kg of the first RNAi agent and about 1.0 mg/kg of the second RNAi agent are administered to a subject in need thereof. In some embodiments, about 3.3 mg/kg of the first RNAi agent and about 1.7 mg/kg of the second RNAi agent are administered to a subject in need thereof. In some embodiments, about 0.05 to about 5 mg/kg of the first RNAi agent and about 0.05 to about 5 mg/kg of the second RNAi agent are administered to a subject in need thereof. In some embodiments, the first RNAi agent and the second RNAi agent are administered separately (e.g., in separate injections). In some embodiments, the respective dose of the first RNAi agent and the respective dose of the second RNAi agent are administered together (e.g., in the same injection). In some embodiments, the respective dose of the first RNAi agent and the respective dose of the second RNAi agent are prepared in a single pharmaceutical composition.

In some embodiment, the RNAi component is administered to the subject once monthly in a dose of about 40-350 mg, such as about 40-250 mg, more particularly 40-200 mg, more particularly 200 mg.

In another aspect, the capsid assembly modulator is a compound of Formula (I):

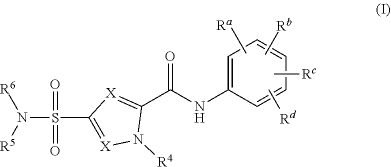

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments, the compound of Formula (I) is a compound of Formula (II):

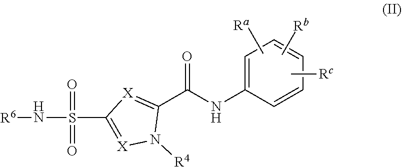

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

In some embodiments of compounds of Formula (I) or (II), R^{a}, R^{b} and R^{c} are independently hydrogen, fluoro, bromo, chloro, or CN. In some embodiments of compounds of Formula (I) or (II), R⁴ is C₁-C₃ alkyl. In some embodiments of compounds of Formula (I) or (II), R⁴ is methyl. In some embodiments of compounds of Formula (I) or (II), R⁶ is C₂-C₆ alkyl optionally substituted with one or more of—OH, fluoro, or C₁-C₄ alkyl optionally substituted with R¹⁰. In some embodiments of compounds of Formula (I) or (II), R⁶ is C₂-C₆ alkyl substituted with one or more fluoro. In some embodiments of compounds of Formula (I) or (II), each R⁷ is independent hydrogen, halogen or methyl. In some embodiments of compounds of Formula (I) or (II), at least one R⁷ is hydrogen.

In another aspect, the CAM compound is selected from the group consisting of:

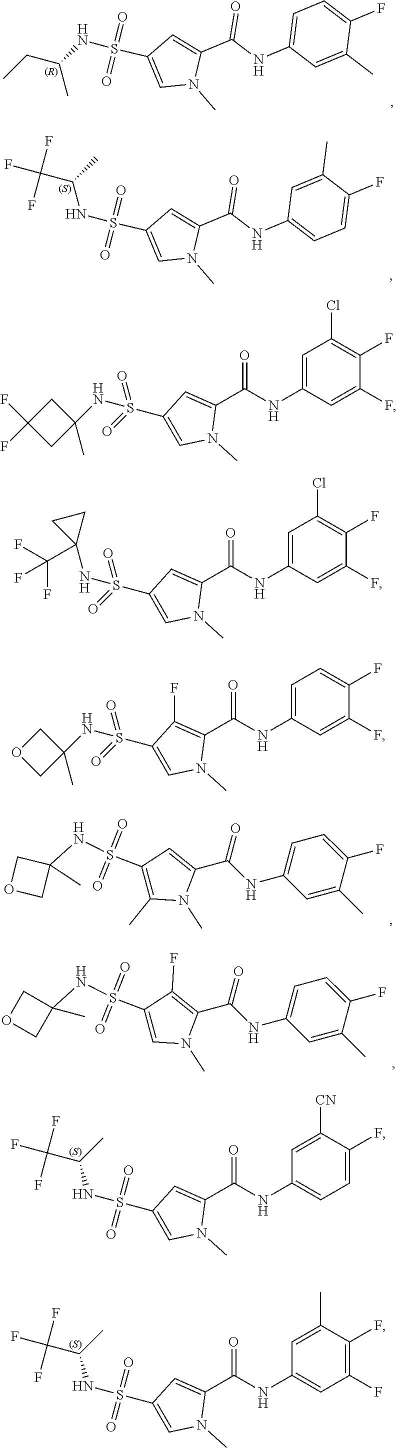

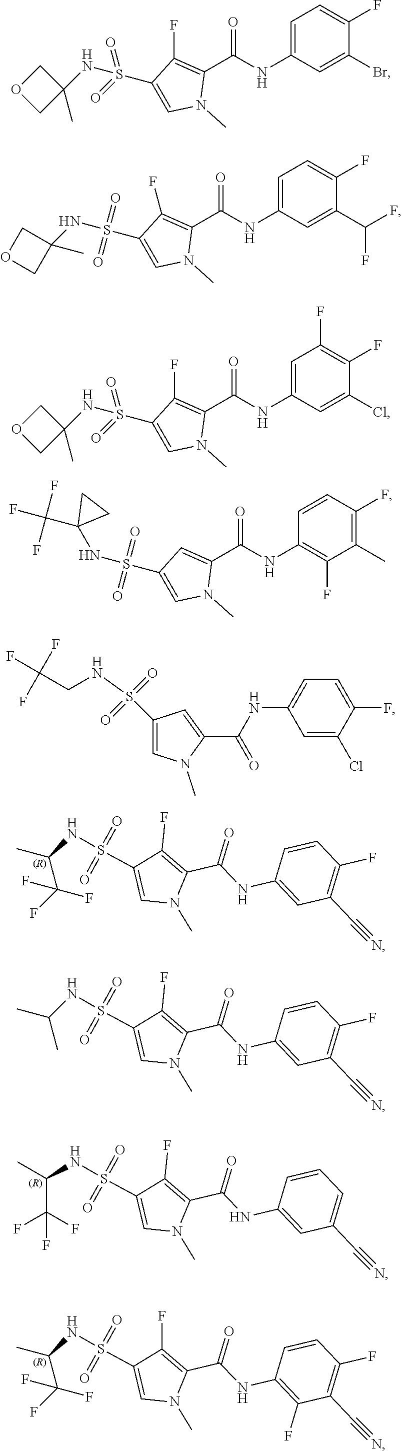

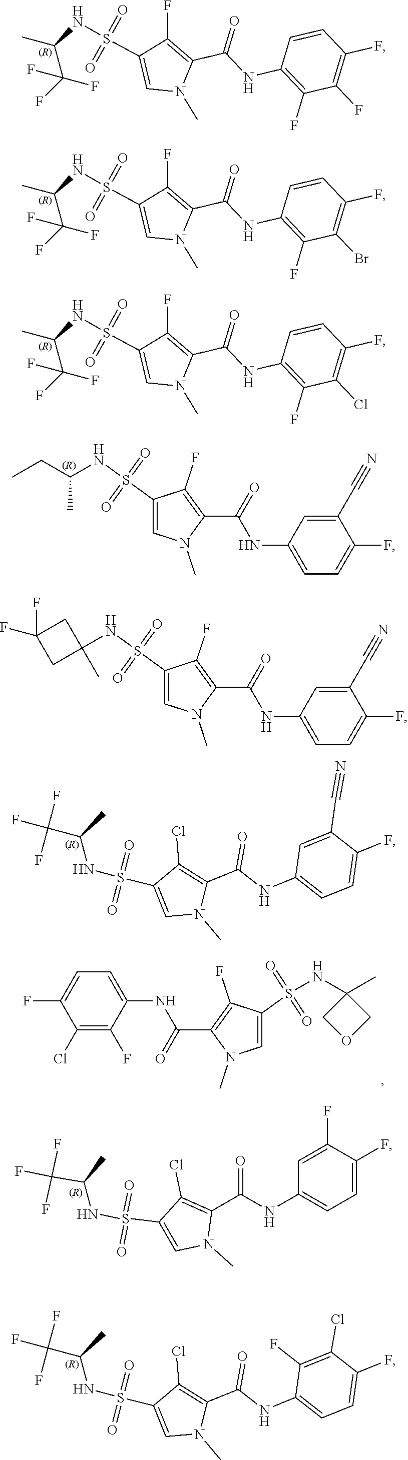

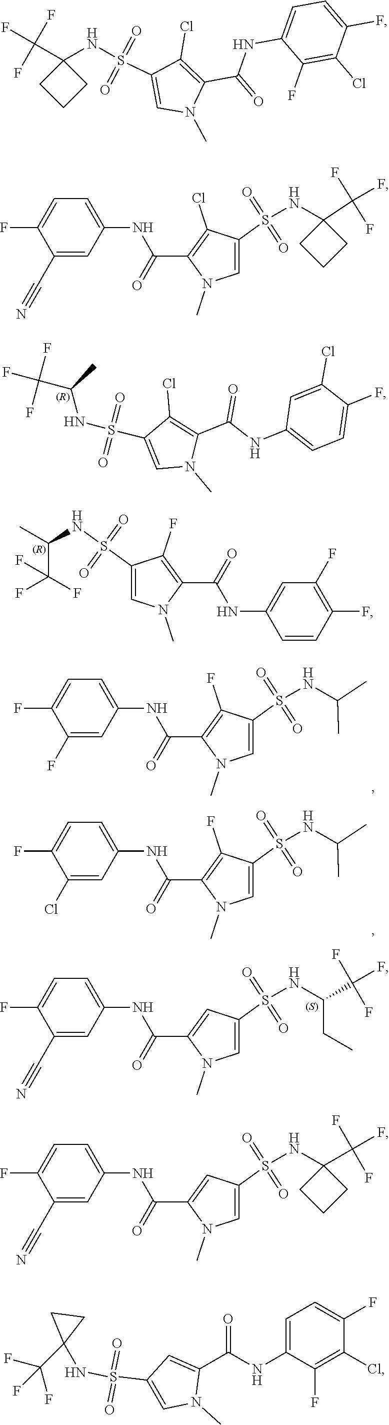

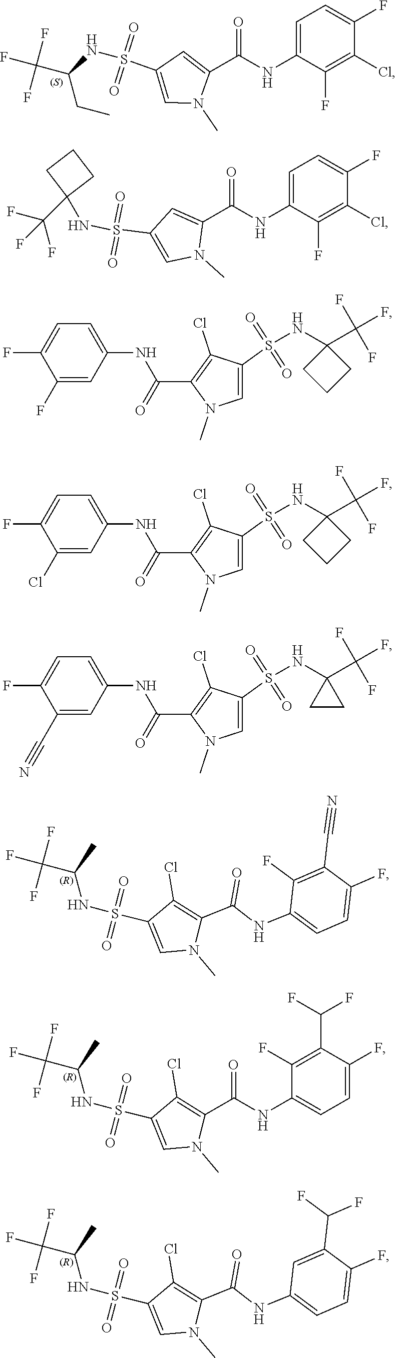

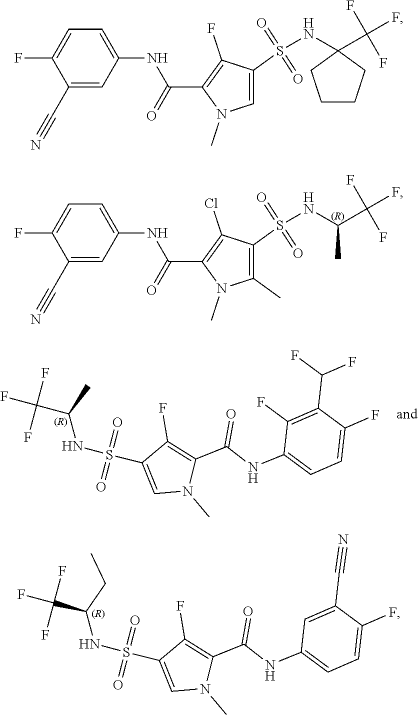

or a pharmaceutically acceptable salt of any of the foregoing.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

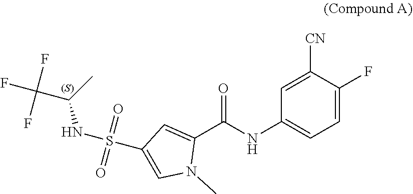

or a pharmaceutically acceptable salt thereof.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

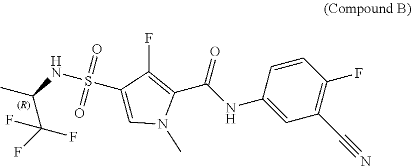

or a pharmaceutically acceptable salt thereof.

In some embodiments, the capsid assembly modulator or a pharmaceutically acceptable salt is administered in the amount of about 50-1000 mg, about 50-75 mg, about 75-100 mg, about 100-150 mg, about 150-200 mg, about 200-250 mg, about 250-300 mg, about 300-350 mg, about 350-400 mg, about 400-450 mg, about 450-500 mg, about 50-750 mg, or about 750-100 mg. In some embodiments, the capsid assembly modulator or a pharmaceutically acceptable salt is administered in the amount of about 100 mg, about 150 mg, about 200 mg, about 250 mg, or about 500 mg. In some embodiments, the capsid assembly modulator or a pharmaceutically acceptable salt is administered in the amount of about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg. In some embodiments, the capsid assembly modulator or a pharmaceutically acceptable salt is administered in the amount of about 100 mg, about 150 mg or about 250 mg. In some embodiments, capsid assembly modulator or a pharmaceutically acceptable salt is administered for a duration of at least about 1 week, at least about 5 weeks, at least about 10 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, or at least about 90 weeks. In some embodiments, capsid assembly modulator or a pharmaceutically acceptable salt is administered for a duration of about 24 weeks or 48 weeks. In some embodiments, the capsid assembly modulator or a pharmaceutically acceptable salt is administered daily, every other day, every week, every 2 weeks, every 3 weeks or every month.

In some embodiments, the capsid assembly modulator or a pharmaceutically acceptable salt is formulated in a solid form, such as a tablet or capsule. In some embodiments, the capsid assembly modulator or a pharmaceutically acceptable salt is formulated in in a liquid form, such as suspensions, solutions, emulsions, or syrups, or may be lyophilized. In some embodiments, the RNAi component is formulated in a solid form, such as a tablet or capsule. In some embodiments, the RNAi component is formulated for subcutaneous injection. In some embodiments, the RNAi component is formulated in in a liquid form, such as suspensions, solutions, emulsions, or syrups, or may be lyophilized.

In some embodiments, the RNAi component and the capsid assembly modulator are administered simultaneously or intermittently. In some embodiments, the RNAi component and the capsid assembly modulator are administered are formulated separately and administered with different dosing frequencies. In some embodiments, the RNAi component and the capsid assembly modulator are formulated as one or separate compositions. In some embodiments, the RNAi component and is formulated as a solution and administered once per month via subcutaneous injection. In some embodiments, the capsid assembly modulator is formulated as an oral tablet and administered daily.

In some embodiments, the RNAi component is administered in the amount of about 50-250 mg once a month via subcutaneous injection while the compound of Formula (I) is administered in the amount of about 100-500 mg daily in the form of a tablet. In some embodiments, the RNAi component is administered in the amount of about 50 mg, about 75 mg, about 100 mg, or about 125 mg. In some embodiments, the compound of Formula (I) is administered in the amount of 150 mg, 200 mg, 250 mg or 300 mg. In some embodiments, the RNAi component is administered in the amount of about 100 mg once a month via subcutaneous injection while the compound of Formula (I) is administered in the amount of about 250 mg daily in the form of a tablet. In some embodiments, the RNAi component comprises a first RNAi agent comprising SEQ ID NO: 2 and SEQ ID NO: 11 and a second RNAi agent comprising SEQ ID NO: 16 and SEQ ID NO: 8 in a ratio of 2:1. In some embodiments, the compound of Formula (I) is compound A:

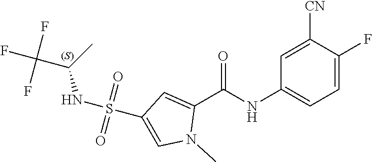

or a pharmaceutically acceptable salt thereof. In some embodiments, the compound of Formula (I) is compound B:

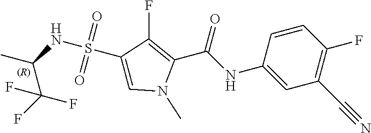

or a pharmaceutically acceptable salt thereof.

In some embodiments, the RNAi component is administered in the amount of about 25-200 mg once a month via subcutaneous injection while the compound of Formula (I) is administered in the amount of about 75-300 mg daily in the form of a tablet. In some embodiments, the RNAi component is administered in the amount of about 35 mg, about 40 mg, about 50 mg, about 100 mg, or about 200 mg. In some embodiments, the compound of Formula (I) is administered in the amount of 75 mg, 150 mg, 250 mg or 300 mg. In some embodiments, the RNAi component is administered in the amount of about 40 mg once a month via subcutaneous injection while the compound of Formula (I) is administered in the amount of about 250 mg daily in the form of a tablet. In some embodiments, the RNAi component is administered in the amount of about 100 mg once a month via subcutaneous injection while the compound of Formula (I) is administered in the amount of about 250 mg daily in the form of a tablet. In some embodiments, the RNAi component is administered in the amount of about 200 mg once a month via subcutaneous injection while the compound of Formula (I) is administered in the amount of about 250 mg daily in the form of a tablet. In some embodiments, the RNAi component comprises a first RNAi agent comprising SEQ ID NO: 2 and SEQ ID NO: 11 and a second RNAi agent comprising SEQ ID NO: 16 and SEQ ID NO: 8 in a ratio of 2:1. In some embodiments, the compound of Formula (I) is compound A:

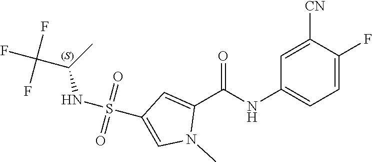

or a pharmaceutically acceptable salt thereof. In some embodiments, the compound of Formula (I) is compound B:

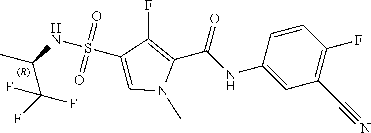

or a pharmaceutically acceptable salt thereof.

In some embodiments, a compound of Formula (I) or a pharmaceutically acceptable salt thereof, such as any of those described herein, is administered to the subject in a daily dose of about 100-500 mg, more particularly of 200-300 mg, more particularly of 250 mg.

In some embodiments, the interferon is pegylated IFN-alpha (e.g., pegylated IFN-alfa 2a or pegylated IFN-alfa 2b) or pegylated IFN-lambda, more particularly pegylated IFN-alfa 2a or pegylated IFN-alpha 2b. Suitable doses of IFN-alpha or IFN-lambda, more particularly of pegylated IFN-alfa (2a or 2b) or pegylated IFN-lambda, more particularly of pegylated IFN-alpha (2a or 2b), include, but are not limited to, 9-75 million Units per week, more particularly 15-35 million Units per week (e.g., 5-10 million Units thrice weekly, or 5 million Units QD, or 10 million Units thrice weekly), or 0.25-4.0 mcg/kg/week (e.g., 1.0-1.5 mcg/kg/week), or 25-360 mcg/QW or 25-360 mcg/BIW, or 25-360 QD (e.g., 100-200 mcg/kg/QW or BIW or QD, e.g., 120-180 mcg/kg/QW or BIW or QD).

In some embodiments, interferon alpha (e.g., Pegasys®, Genentech) is administered weekly. In some embodiments, the interferon alpha is administered at a dose of 120 mcg QW or 180 mcg QW. In some embodiments, pegylated interferon (Pegasys®) is administered at a dose of 180 micrograms per week.

In one embodiment of these combination therapies, pegylated interferon alfa-2a (Pegasys) is administered weekly in dosages of 180 microgram (meg) or 120 mcg or 135 meg (used for patients that react negatively to the higher dose) subcutaneously (SQ).

In another embodiment of these combination therapies, pegylated interferon alfa-2b (PegIntron) is administered weekly in dosages of 1.5 meg/kg/wk SQ.

In other embodiments of these methods, alfa-interferons are used as follows: consensus interferon (Infergen) administered at 9 mcg to 15 mcg SQ daily or thrice weekly; interferon-alfa 2a recombinant administered at 3 Ml U to 9 MIU SQ administered thrice weekly; interferon-alfa 2b (Intron A) recombinant administered 3 MIU to 25 MIU SQ administered thrice weekly; and pegylated interferon lambda (IL-28) administered at 80 meg to 240 mcg SQ weekly.

In some embodiment, an interferon is administered to the subject by intravenous or subcutaneous injection in a dose of about 25-500 meg, preferably 80-300 meg, per week, preferably once per week, more particularly of 100-200 mcg per week, more particularly 180 meg per week.

In some embodiments, the interferon is pegylated IFN lambda-1a. Suitable doses of pegylated IFN lambda-1a include, but are not limited to, 180 mcg QW; 120 mcg QW; 180 mcg/BIW; 120 mcg/BIW.

In some embodiments, interferon lambda (e.g., pegylated lambda, e.g., pegylated lambda-1a) is administered weekly. In some embodiments, the interferon lambda is administered at a dose of 120 mcg QW or 180 mcg QW. In some embodiments, the interferon lambda is administered at a dose of 120 micrograms per week.

In some embodiments, interferon lambda is administered at a dose of 120 micrograms per week. In some embodiments, interferon lambda is administered at a dose of 180 micrograms per week. In some embodiments, the interferon lambda is subcutaneously administered.

In some embodiments, the method further comprises administering a nucleoside analog. In some embodiments, the nucleoside analog is entecavir, tenofovir disoproxil fumarate or tenofovir alafenamide. In some embodiments, the nucleoside analog is entecavir and it is administered in the amount of about 0.01-5 mg, about 0.01-0.05 mg, about 0.05-0.1 mg, about 0.1-0.5 mg, about 0.5-1 mg, about 1-2 mg, about 2-3 mg, about 3-4 mg or about 4-5 mg. In some embodiments, the nucleoside analog is entecavir and it is administered in the amount of about 0.5 mg. In some embodiments, the nucleoside analog is tenofovir disoproxil fumarate and it is administered in the amount of about 100-500 mg, about 100-150 mg, about 150-200 mg, about 200-250 mg, about 250-300 mg, 300-400 mg, about 400-500 mg. In some embodiments, the nucleoside analog is tenofovir disoproxil fumarate and it is administered in the amount of about 300 mg. In some embodiments, the nucleoside analog is tenofovir alafenamide and it is administered in the amount of about 5-100 mg, about 5-25 mg, about 25-50 mg, about 50-75 or about 75-100 mg. In some embodiments, the nucleoside analog is tenofovir alafenamide and it is administered in the amount of about 25 mg. In some embodiments, the patients have been exposed to the nucleoside analog prior to the combination therapy. In some embodiments, the patients have been administered the nucleoside analog for at least 1 month, at least 3 months, at least 6 months, or at least 1 year prior to receiving the combination therapy.

In some embodiments, interferon alpha or lambda therapy is administered in conjunction with an RNAi component, a compound of formula (I), and optionally a nucleoside analog, for treating a HBV infection in a patient for at least 1 month, 2 months, 3 months, 4 months, 5 months, 6 months or longer.

In some embodiments, the patients are screened for HBeAg status prior to administration of the combination therapy. In some embodiments, the patients are HBeAg positive. In some embodiments, the patients are HBeAg negative. In some embodiment, the patients are screened for immune tolerance prior to administration of the combination therapy.

In some embodiments, the HBsAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from baseline on Day 1. In some embodiments, the HBeAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBcrAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from baseline on Day 1. In some embodiments, the HBV DNA level in the patient is reduced by at least about log₁₀ 0.5, about log₁₀ 1, about log₁₀ 1.5, about log₁₀ 2, about log₁₀ 3, about log₁₀ 4, about log₁₀ 5 or about log₁₀ 7.5 from baseline on Day 1. In some embodiments, the HBV RNA level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from baseline on Day 1.

In some embodiments, the patients have been receiving a capsid assembly modulator for at least about 1 month, about 3 months, about 6 months or about 1 year before the administration of the RNAi component and the interferon.

In some embodiments, the patients have been receiving a treatment with an interferon for at least about 1 month, about 3 months, about 6 months or about 1 year before the administration of the RNAi component and the capsid assembly modulator of formula (I).

The application also relates to an effective amount of an RNAi component, a compound of Formula (I) and an interferon, optionally a nucleoside analog, each of which as that described herein, in the manufacture of a medicament for treating a viral infection in a subject caused by Hepatitis B Virus. In some embodiment, the RNAi component is for once monthly administration to a subject in a dose of about 40-250 mg, more particularly 40-200 mg, more particularly 200 mg; the compound of Formula (I) or a pharmaceutically acceptable salt thereof is for daily administration to a subject in a dose of about 100-500 mg, particularly of 200-300 mg, more particularly of 250 mg; and the interferon is for intravenous or subcutaneous injection in a dose of about 25-500 meg, preferably 80-300 meg, per week, preferably once per week, more particularly of 100-200 mcg per week, more particularly 180 meg per week. In some embodiment, the medicament is for administering to a subject infected by HBV, in particular, a subject having a chronic HBV infection, more particularly the subject is infected by HBV but is immune tolerant.

The application includes, but is not limited to, the following embodiments:

1. A method for inhibiting the expression of a Hepatitis B Virus gene in a subject in need thereof, wherein the method comprises administering to the subject:

(a) an effective amount of a pharmaceutical composition comprising an RNAi component having:
(i) a first RNAi agent comprising: an antisense strand comprising a nucleotide sequence of any one of the following: SEQ ID NOT, SEQ ID NO:2, SEQ ID NOT, SEQ ID NO:4, SEQ ID NOT, SEQ ID NO:6, and SEQ ID NO:7 and a sense strand comprising a nucleotide sequence of any one of the following: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NOT and SEQ ID NOV, and a sense strand comprising a nucleotide sequence of any one of the following: SEQ ID NO: 16, SEQ ID NO: 17, SEQ ID NO: 18, and SEQ ID NO: 19; and

(b) an effective amount of a pharmaceutical composition comprising a compound of Formula (I):

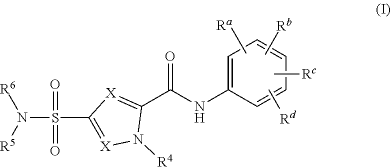

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and
(c) an effective amount of a pharmaceutical composition comprising an interferon, such as an interferon alpha or lambda, preferably a pegylated interferon, more preferably a pegylated interferon alpha-2a or pegylated interferon lambda-1a.
   2. A method for treating a disease or disorder associated with an infection caused by a Hepatitis B Virus in a subject, wherein the method comprises administering to the subject:

(a) an effective amount of a pharmaceutical composition comprising an RNAi component as described in Embodiment 1 (a);

(b) an effective amount of a pharmaceutical composition comprising a compound of Formula (I) or a pharmaceutically acceptable salt thereof as described in Embodiment 1 (b); and

(c) an effective amount of a pharmaceutical composition comprising an interferon as described in Embodiment 1 (c).

3. A method of treating an HBV infection or a disease or disorder associated with an infection caused by Hepatitis B Virus in a subject receiving a capsid assembly modulator therapy, wherein the method further comprises administering to the subject an effective amount of an RNAi component as described in Embodiment 1 (a) and an interferon as described in Embodiment 1 (c).
4. The method of embodiment 3, wherein the capsid assembly modulator is a compound of Formula (I) or a pharmaceutically acceptable salt thereof as described in Embodiment 1 (b).
5. A method of treating HBV or a disease or disorder associated with an infection caused by Hepatitis B Virus in a subject receiving an interferon therapy, wherein the method further comprises administering to the subject an effective amount of an RNAi component as described in Embodiment 1 (a) and a capsid assembly modulator comprising a compound of Formula (I) or a pharmaceutically acceptable salt thereof as described in Embodiment 1 (b).
6. The method of embodiment 5, wherein the interferon is an interferon alpha or lambda, preferably a pegylated interferon, more preferably a pegylated interferon alpha-2a or pegylated interferon lambda-1a.

7. A method for treating HBV or a disease or disorder associated with an infection caused by Hepatitis B Virus in a subject receiving a nucleoside analog or nucleotide analog therapy, wherein the method comprises administering to the subject:

(a) an effective amount of a pharmaceutical composition comprising an RNAi component as described in Embodiment 1 (a); and

(b) an effective amount of a pharmaceutical composition comprising a compound of Formula (I) or a pharmaceutically acceptable salt thereof as described in Embodiment 1 (b); and

(c) an effective amount of a pharmaceutical composition comprising an interferon, as described in Embodiment 1 (c).

8. The method of embodiment 7, wherein the nucleoside analog or nucleotide analog is selected from entecavir, tenofovir disoproxil fumarate, tenofovir disoproxil maleate, and tenofovir alafenamide.
9. A method of treating a Hepatitis B Virus infection in a subject in need thereof, comprising contacting a cell infected with the Hepatitis B Virus with an effective amount of an RNAi component as described in Embodiment 1 (a), a capsid assembly modulator comprising a compound of Formula (I) or a pharmaceutically acceptable salt thereof as described in Embodiment 1 (b), and an interferon as described in Embodiment 1 (c).
9a. A combination for use in treating an HBV infection or a disease or disorder associated with an HBV infection in a subject in need thereof, comprising:

(a) a first pharmaceutical composition comprising an RNAi component as described in Embodiment 1 (a); and

(b) a second pharmaceutical composition comprising a compound of Formula (I) or a pharmaceutically acceptable salt thereof as described in Embodiment 1 (b); and

(c) a third pharmaceutical composition comprising an interferon as described in Embodiment 1 (c).

9b. A first pharmaceutical composition for use in a treatment of an HBV infection or a disease or disorder associated with an HBV infection in a subject in need thereof, wherein the first pharmaceutical composition comprises a RNAi component as described in Embodiment
1 (a), and wherein the treatment comprises:
(a) the administration of the first pharmaceutical composition,
(b) the administration of a second pharmaceutical composition comprising a compound of formula (I) or a pharmaceutically acceptable salt thereof as described in Embodiment 1 (b), and
(c) the administration of a third pharmaceutical composition comprising an interferon as described in Embodiment 1 (c).
   9b 1. A first pharmaceutical composition for use in a combination with a second pharmaceutical composition and a third pharmaceutical composition for treating an HBV infection or a disease or disorder associated with the HBV infection in a subject in need thereof, wherein:
(a) the first pharmaceutical composition comprises an RNAi component as described in Embodiment 1 (a);
(b) the second pharmaceutical composition comprises a compound of Formula (I) or a pharmaceutically acceptable salt thereof as described in Embodiment 1 (b); and
(c) the third pharmaceutical composition comprises an interferon as described in Embodiment 1 (c).
   9b2. An RNAi component for use in a treatment of an HBV infection, more particularly a chronic HBV infection, in a subject in need thereof,
   wherein the treatment comprises administering to the subject the RNAi component in combination with a compound of formula (I) or a pharmaceutically acceptable salt thereof and in combination with interferon and/or
   the RNAi component is for administration to the subject in combination with the compound of formula (I) or the pharmaceutically acceptable salt thereof and in combination with interferon,
   wherein the RNAi component, the compound of formula (I) or the pharmaceutically acceptable salt thereof and the interferon are as described in embodiment 1.
   9c. A second pharmaceutical composition comprising a compound of Formula (I) or a pharmaceutically acceptable salt thereof as described in Embodiment 1 (b) for use in a treatment of an HBV infection or a disease or disorder associated with an HBV infection in a subject in need thereof, wherein the treatment comprises:
(a) the administration of the second pharmaceutical composition;
(b) the administration of a first pharmaceutical composition comprising a RNAi component as described in Embodiment 1 (a), and
(c) the administration of a third pharmaceutical composition comprising an interferon as described in Embodiment 1 (c).
   9c1. A second pharmaceutical composition for use in a combination with a first pharmaceutical composition and a third pharmaceutical composition for treating an HBV infection or a disease or disorder associated with the HBV infection in a subject in need thereof, wherein:
(a) the first pharmaceutical composition comprises an RNAi component as described in Embodiment 1 (a);
(b) the second pharmaceutical composition comprises a compound of Formula (I) or a pharmaceutically acceptable salt thereof as described in Embodiment 1 (b); and
(c) the third pharmaceutical composition comprises an interferon as described in Embodiment 1 (c).
   9c2. A compound of formula (I) or a pharmaceutically acceptable salt thereof for use in a treatment of an HBV infection, more particularly a chronic HBV infection, in a subject in need thereof,
   wherein the treatment comprises administering to the subject the compound of formula (I) or the pharmaceutically acceptable salt thereof in combination with an RNAi component and in combination with interferon and/or
   the compound of formula (I) or the pharmaceutically acceptable salt thereof is for administration to the subject in combination with the RNAi component and in combination with interferon,
   wherein the RNAi component, the compound of formula (I) or the pharmaceutically acceptable salt thereof and the interferon are as described in embodiment 1.
   9d. A third pharmaceutical composition comprising an interferon as described in embodiment 1 (c) for use in a treatment of an HBV infection or a disease or disorder associated with an HBV infection in a subject in need thereof, wherein the treatment comprises:
(a) the administration of the third pharmaceutical composition,
(b) the administration of a first pharmaceutical composition comprising a RNAi component as described in Embodiment 1 (a); and
(c) the administration of a second pharmaceutical composition comprising a compound of Formula (I) or a pharmaceutically acceptable salt thereof as described in Embodiment 1 (b).
   9d1. A third pharmaceutical composition for use in a combination with a first pharmaceutical composition and a second pharmaceutical composition for treating an HBV infection or a disease or disorder associated with the HBV infection in a subject in need thereof, wherein:
(a) the first pharmaceutical composition comprises an RNAi component as described in Embodiment 1 (a);
(b) the second pharmaceutical composition comprises a compound of Formula (I) or a pharmaceutically acceptable salt thereof as described in Embodiment 1 (b); and
(c) the third pharmaceutical composition comprises an interferon as described in Embodiment 1 (c).
   9d2. An interferon for use in a treatment of an HBV infection, more particularly a chronic HBV infection, in a subject in need thereof,
   wherein the treatment comprises administering to the subject the interferon in combination with a RNAi component and in combination with a compound of formula (I) or a pharmaceutically acceptable salt thereof, and/or
   the interferon is for administration to the subject in combination with the compound of formula (I) or the pharmaceutically acceptable salt thereof and in combination with the RNAi component,
   wherein the RNAi component, the compound of formula (I) or the pharmaceutically acceptable salt thereof and the interferon are as described in embodiment 1.
   10. The method, or the combination, pharmaceutical composition, RNAi component, compound or interferon for use of any one of embodiments 1-9d2, wherein the first or the second RNAi agent comprises at least one modified nucleotide or at least one modified internucleoside linkage.
   11. The method, or the combination, pharmaceutical composition, RNAi component, compound or interferon for use of any one of embodiments 1-10, wherein substantially all of the nucleotides in the first and the second RNAi agents are modified nucleotides.
   12. The method, or the combination, pharmaceutical composition, RNAi component, compound or interferon for use of any one of embodiments 1-11, wherein the first or the second RNAi agent further comprises a targeting ligand that is conjugated to the first or the second RNAi agent.
   13. The method, or the combination, pharmaceutical composition, RNAi component, compound or interferon for use of embodiment 12, wherein the targeting ligand comprises N-acetyl-galactosamine.
   14. The method, or the combination, pharmaceutical composition, RNAi component, compound or interferon for use of embodiment 13, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.
   15. The method, or the combination, pharmaceutical composition, RNAi component, compound or interferon for use of embodiment 14, wherein the targeting ligand is (NAG25), (NAG25)s, (NAG31), (NAG31)s, (NAG37), or (NAG37)s.
   16. The method, or the combination, pharmaceutical composition, RNAi component, compound or interferon for use of any one of embodiments 12-15, wherein the targeting ligand is conjugated to the sense strand of the first or the second RNAi agent.
   17. The method, or the combination, pharmaceutical composition, RNAi component, compound or interferon for use of embodiment 16, wherein the targeting ligand is conjugated to the 5′ terminus of the sense stand of the first or the second RNAi agent.
   18. The method, or the combination, pharmaceutical composition, RNAi component, compound or interferon for use of any one of embodiments 1-17, wherein the first and the second RNAi agents independently comprise a duplex selected from the group consisting of: an antisense strand comprising SEQ ID NO: 5 and a sense strand comprising SEQ ID NO: 14;
an antisense strand comprising SEQ ID NO: 6 and a sense strand comprising SEQ ID NO: 14;
an antisense strand comprising SEQ ID NO: 7 and a sense strand comprising SEQ ID NO: 15; and
an antisense strand comprising SEQ ID NO: 9 and a sense strand comprising SEQ ID NO: 19.
   18a. The method, or the combination, pharmaceutical composition, RNAi component, compound or interferon for use of any one of embodiments 1-18, wherein the first and the second RNAi agents independently comprise a duplex selected from the group consisting of: an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO: 18.
   19. The method, or the combination, pharmaceutical composition, RNAi component, compound or interferon for use of any one of embodiments 1-18a, wherein the first and the second RNAi agents are each independently conjugated to a targeting ligand comprising N-acetyl-galactosamine, and the first and the second RNAi agents independently comprise a duplex selected from the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.
   20. The method, or the combination, pharmaceutical composition, RNAi component, compound or interferon for use of any one of embodiments 1-19, wherein the ratio of the first RNAi agent to the second RNAi agent by weight is in the range of about 1:2 to about 5:1.
   21. The method, or the combination, pharmaceutical composition, RNAi component, compound or interferon for use of embodiment 20, wherein the ratio of the first RNAi agent to the second RNAi agent by weight is about 2:1.
   22. The method, or the combination, pharmaceutical composition, RNAi component, compound or interferon for use of any one of embodiments 1, 2 and 4-21, wherein the compound of Formula (I) is a compound of Formula (II):

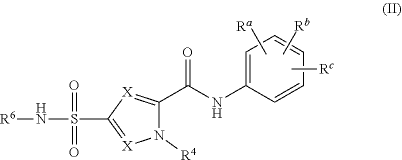

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.
   23. The method, or the combination, pharmaceutical composition, RNAi component, compound or interferon for use of embodiment 22, wherein R^{a}, R^{b} and R^{c} are independently hydrogen, fluoro, bromo, chloro, or CN.
   24. The method, or the combination, pharmaceutical composition, RNAi component, compound or interferon for use of embodiment 22 or 23, wherein R⁴ is C₁-C₃ alkyl.
   25. The method, or the combination, pharmaceutical composition, RNAi component, compound or interferon for use of embodiment 24, wherein R⁴ is methyl.

26. The method, or the combination, pharmaceutical composition, RNAi component, compound or interferon for use of any one of embodiments 22-25, wherein R⁶ is C₂-C₆ alkyl optionally substituted with one or more of—OH, fluoro, or C₁-C₄ alkyl optionally substituted with R¹⁰.

27. The method, or the combination, pharmaceutical composition, RNAi component, compound or interferon for use of embodiment 26, wherein R⁶ is C₂-C₆ alkyl substituted with one or more fluoro.

28. The method, or the combination, pharmaceutical composition, RNAi component, compound or interferon for use of any one of embodiments 22-27, wherein each R⁷ is independent hydrogen, halogen or methyl.

29. The method, or the combination, pharmaceutical composition, RNAi component, compound or interferon for use of embodiment 28, wherein at least one R⁷ is hydrogen.

30. The method, or the combination, pharmaceutical composition, RNAi component, compound or interferon for use of any one of embodiments 1, 2 and 4-21, wherein the compound of Formula (I) is selected from the group consisting of:

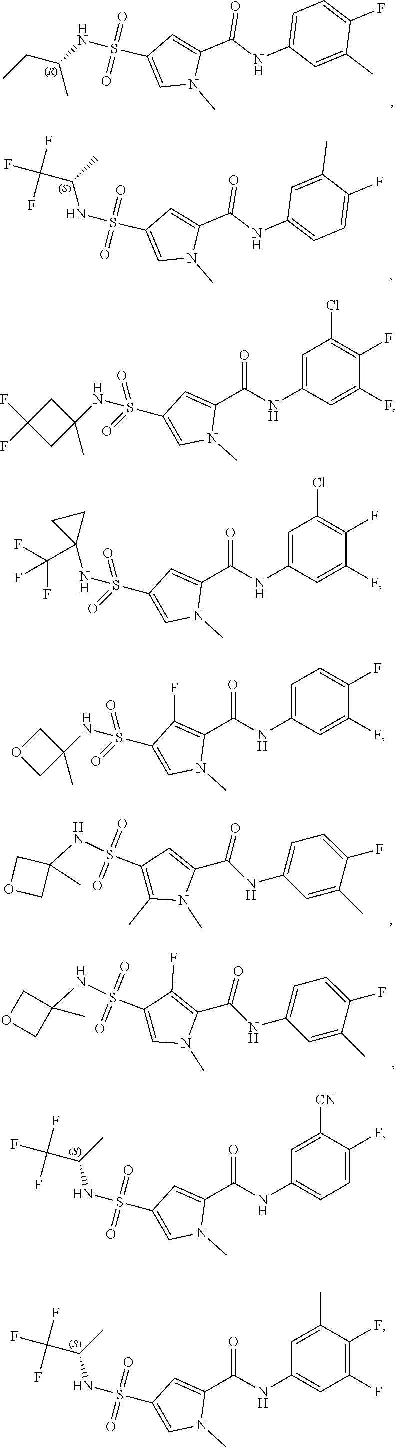

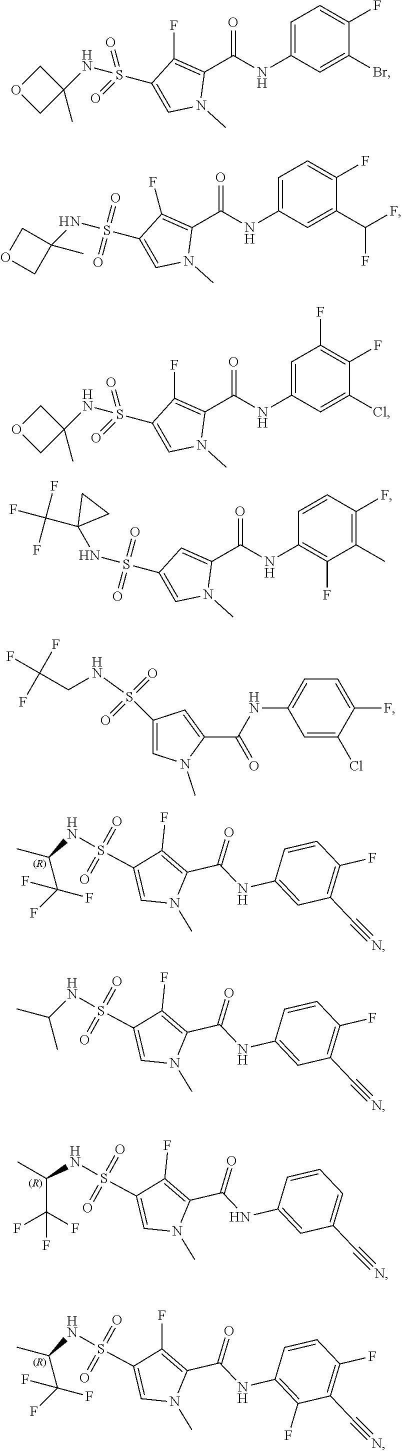

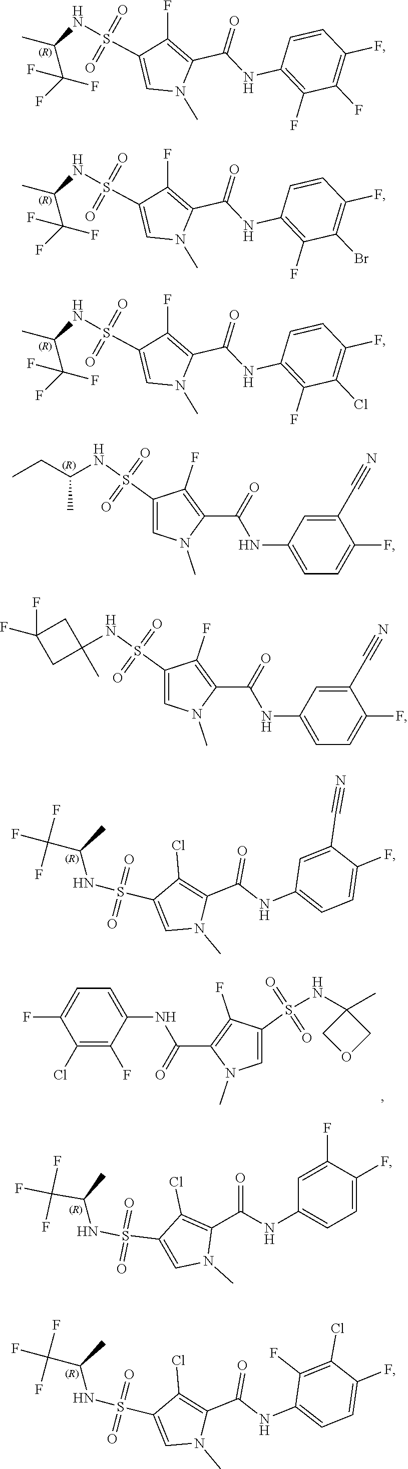

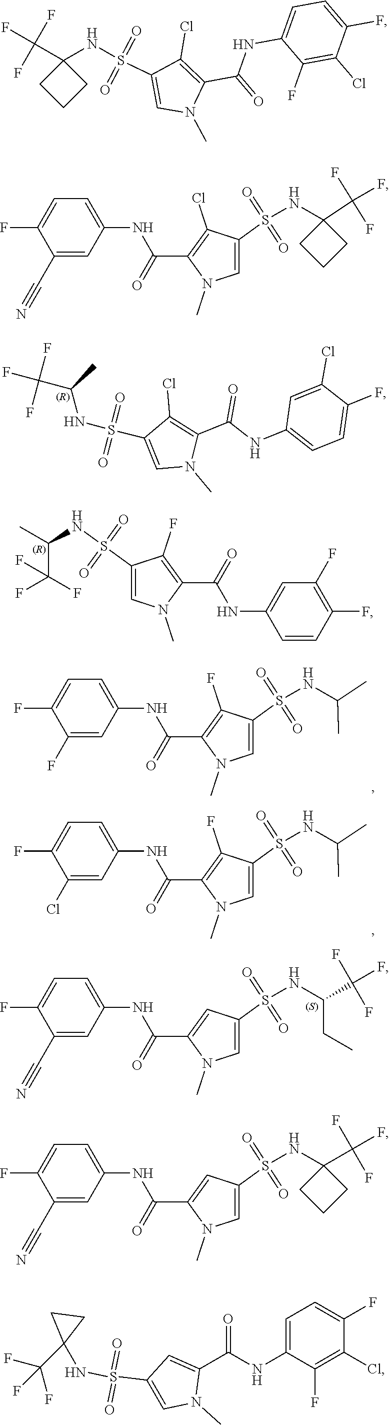

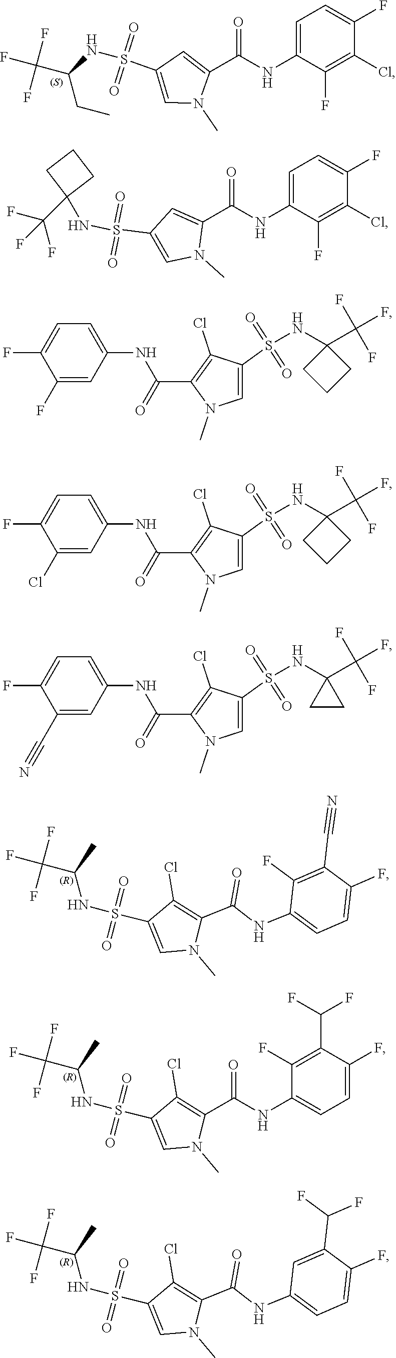

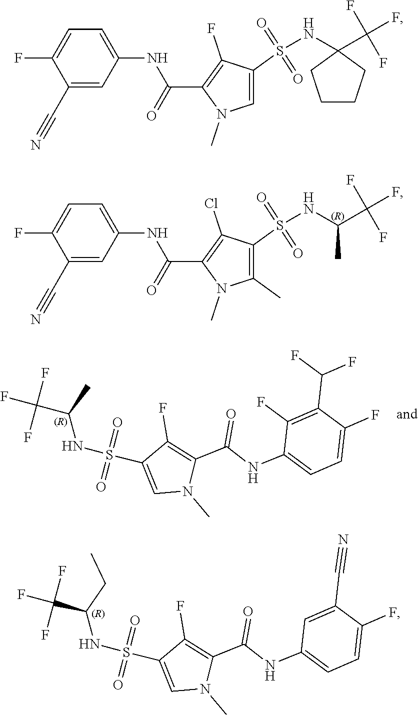

or a pharmaceutically acceptable salt of any of the foregoing.
31. The method, or the combination, pharmaceutical composition, RNAi component, compound or interferon for use of embodiment 30, wherein the compound of Formula (I) is

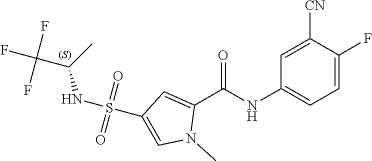

or a pharmaceutically acceptable salt thereof.
32. The method, or the combination, pharmaceutical composition, RNAi component, compound or interferon for use of embodiment 30, wherein the compound of Formula (I) is

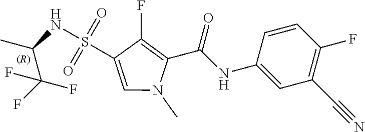

or a pharmaceutically acceptable salt thereof.
33. The method, or the combination, pharmaceutical composition, RNAi component, compound or interferon for use of any one of embodiments 1-9d2, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) or the capsid assembly modulator is

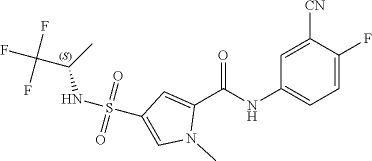

or a pharmaceutically acceptable salt thereof, and the interferon is pegylated interferon alpha-2a or pegylated interferon lambda-1a.
34. The method, or the combination, pharmaceutical composition, RNAi component, compound or interferon for use of any one of embodiments 1-9d2, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) or the capsid assembly modulator is

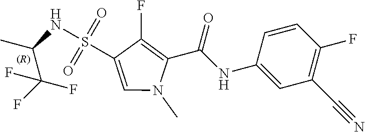

or a pharmaceutically acceptable salt thereof, and the interferon is pegylated interferon alpha-2a or pegylated interferon lambda-1a.
35. The method, or the combination, pharmaceutical composition, RNAi component, compound or interferon for use of any one of embodiments 1-34, wherein the RNAi component is administered to the subject once monthly in a dose of about 40-250 mg, more particularly 40-200 mg, more particularly 200 mg.
36. The method, or the combination, pharmaceutical composition, RNAi component, compound or interferon for use of any one of embodiments 1, 2 and 4-35, wherein the compound of Formula (I) or a pharmaceutically acceptable salt thereof is administered to the subject in a daily dose of about 100-500 mg, more particularly of 200-300 mg, more particularly of 250 mg.
37. The method, or the combination, pharmaceutical composition, RNAi component, compound or interferon for use of any one of embodiments 1-36, wherein the RNAi component is administered to the subject via intravenous or subcutaneous injection.
38. The method, or the combination, pharmaceutical composition, RNAi component, compound or interferon for use of any one of embodiments 1, 2 and 4-37, wherein the compound of Formula (I) or a pharmaceutically acceptable salt thereof is administered to the subject orally.
39. The method, or the combination, pharmaceutical composition, RNAi component, compound or interferon for use of any one of embodiments 1-38, wherein the interferon is administered to the subject in a dose of about 25-500 meg, preferably 80-300 meg, per week, preferably once per week, more particularly of 100-200 mcg per week, more particularly 180 meg per week.
40. The method, or the combination, pharmaceutical composition, RNAi component, compound or interferon for use of any one of embodiments 1-39, wherein the interferon is administered to the subject via intravenous or subcutaneous injection.
41. The method, or the combination, pharmaceutical composition, RNAi component, compound or interferon for use of any one of embodiments 1-40, wherein the RNAi component is administered simultaneously or sequentially with the capsid assembly modulator or the compound of Formula (I) or a pharmaceutically acceptable salt thereof.
42. The method, or the combination, pharmaceutical composition, RNAi component, compound or interferon for use of any one of embodiments 1-40, wherein the RNAi component is administered separately from the capsid assembly modulator or the compound of Formula (I) or a pharmaceutically acceptable salt thereof.
43. The method, or the combination, pharmaceutical composition, RNAi component, compound or interferon for use of any one of embodiments 1-42, wherein the subject has been receiving the capsid assembly modulator therapy or the compound of Formula (I) or a pharmaceutically acceptable salt thereof for at least about 1 month.
44. The method, or the combination, pharmaceutical composition, RNAi component, compound or interferon for use of any one of embodiments 1-43 further comprising administering to the subject a nucleoside analog or a nucleotide analog.
45. The method, or the combination, pharmaceutical composition, RNAi component, compound or interferon for use of embodiment 44, wherein the nucleoside analog is entecavir, tenofovir disoproxil fumarate, tenofovir disoproxil maleate, or tenofovir alafenamide.
46. The method, or the combination, pharmaceutical composition, RNAi component, compound or interferon for use of embodiment 45, wherein entecavir is administered to the subject in a daily dose of about 0.1-5 mg.
47. The method, or the combination, pharmaceutical composition, RNAi component, compound or interferon for use of embodiment 45, wherein tenofovir is administered to the subject in a daily dose of about 5-50 mg of tenofovir alafenamide or about 200-500 mg of tenofovir disoproxil fumarate or tenofovir disoproxil maleate.
48. The method, or the combination, pharmaceutical composition, RNAi component, compound or interferon for use of any one of embodiments 1-47, wherein the subject has a chronic HBV infection, more particularly the subject is infected by HBV but is immune tolerant.
49. The method, or the combination, pharmaceutical composition, RNAi component, compound or interferon for use of any one of embodiments 1-48, wherein the method comprises administering the effective amount the pharmaceutical composition comprising the RNAi component, the effective amount of the pharmaceutical composition comprising the compound of formula (I), and the effective amount of the pharmaceutical composition comprising the interferon, until the subject meets with at least one of, at least two of, at least three of, or the four of following features:

i. a serum HBV DNA lower than the lower limit of quantification (LLoQ) or is lower than 20 IU/mL, more particularly is lower than 20 IU/mL, more particularly is lower than 15 IU/mL,

ii. a serum ALT concentration lower than 3 times the upper normal limit, or lower than 129 U/L if the subject is a male subject, or lower than 108 U/L if the subject is a female subject, more particularly a serum ALT concentration lower than 120 U/L if the subject is a male subject or lower than 105 U/L if the subject is a female subject, more particularly a serum ALT concentration lower than 90 U/L if the subject is a male subject or lower than 57 U/L if the subject is a female subject,

iii. a HBeAg-negative serum, and

iv. a serum HBsAg of 100 IU/mL or lower, more particularly of 10 IU/mL or lower.

50. The method, or the combination, pharmaceutical composition, RNAi component, compound or interferon for use of any one of embodiments 1-49, wherein the method comprises administering to the subject the effective amount the pharmaceutical composition comprising the RNAi component, the effective amount of the pharmaceutical composition comprising the compound of formula (I), and the effective amount of the pharmaceutical composition comprising the interferon, for 10-80 weeks, more particularly 12-72 weeks, more particularly 12-60 weeks, more particularly 12-48 weeks, more particularly 12 weeks.
51. The method, or the combination, pharmaceutical composition, RNAi component, compound or interferon for use of embodiment 49 or 50, further comprising administering to the subject a nucleoside analog or a nucleotide analog, such as entecavir, tenofovir disoproxil fumarate, tenofovir disoproxil maleate, or tenofovir alafenamide, and wherein the administration of the nucleoside or nucleotide analog is optionally being continued once the administration of the effective amount the pharmaceutical composition comprising the RNAi component, the effective amount of the pharmaceutical composition comprising the compound of formula (I) or the pharmaceutically acceptable salt thereof, and the effective amount of the pharmaceutical composition comprising the interferon stops.
52. The method, or the combination, pharmaceutical composition, RNAi component, compound or interferon for use of any one of embodiments 1-51, wherein, prior to administering the effective amount of the pharmaceutical composition comprising the interferon, the subject is administered with the pharmaceutical composition comprising the RNAi component and the pharmaceutical composition comprising the compound of formula (I) or the pharmaceutically acceptable salt thereof until the HBsAg concentration of the serum of the subject is 100 IU/mL or lower, more particularly 10 IU/mL or lower, and/or for a duration of 10-60 weeks, more particularly 36-60 weeks.
53. The method or the combination, pharmaceutical composition, RNAi component, compound or interferon for use of embodiment 52, wherein the administration of the pharmaceutical composition comprising the RNAi component and the pharmaceutical composition comprising the compound of formula (I) or the pharmaceutically acceptable salt thereof stops once the HBsAg concentration of the serum of the subject is 100 IU/mL or lower, more particularly 10 IU/mL or lower, and/or once the duration of the administration of the pharmaceutical composition comprising the RNAi component and the pharmaceutical composition comprising the compound of formula (I) totalizes 60 weeks.
54. The method or the combination, pharmaceutical composition, RNAi component, compound or interferon for use of embodiment 52, wherein the administrations of the first pharmaceutical composition comprising the RNAi component and the second pharmaceutical composition comprising the compound of formula (I) or the pharmaceutically acceptable salt thereof continue after the administration of the third pharmaceutical composition comprising the interferon, in particular, the administrations of the first, second and third pharmaceutical compositions continue for 12 weeks after the initial administration of the third pharmaceutical composition.
55. The method, or the combination, pharmaceutical composition, RNAi component, compound or interferon for use of embodiment 52, 53 or 54, further comprising administering to the subject a nucleoside analog or a nucleotide analog, such as entecavir, tenofovir disoproxil fumarate, tenofovir disoproxil maleate, or tenofovir alafenamide.
56. The method, or the combination, pharmaceutical composition, RNAi component, compound or interferon for use of embodiment of any one of claim 1-55, wherein the RNAi component is for once monthly intravenous or subcutaneous administration to a subject in a dose of about 40-250 mg, more particularly 40-200 mg, more particularly 200 mg; the compound of Formula (I) or a pharmaceutically acceptable salt thereof is for daily oral administration to a subject in a dose of about 100-500 mg, particularly of 200-300 mg, more particularly of 250 mg; and the interferon is for intravenous or subcutaneous injection in a dose of about 25-500 meg, preferably 80-300 meg, per week, preferably once per week, more particularly of 100-200 mcg per week, more particularly 180 mcg per week.
57. The method, or the combination, pharmaceutical composition, RNAi component, compound or interferon for use of any one of embodiments 1-56, wherein the disease or disorder associated with the HBV infection comprises one or more selected from the group consisting of jaundice, fulminant hepatitis, liver injury, liver failure, end-stage liver disease, cirrhosis, and liver cancer (such as hepatocellular carcinoma).
58. The method, or the combination, pharmaceutical composition, RNAi component, compound or interferon for use of embodiment 57, wherein the subject is infected with an HBV, more particularly the subject has a chronic HBV infection, even more particularly, the subject has a chronic HBV infection and another infection (more particularly chronic infection) with at least one of: hepatitis D virus (HDV); hepatitis C virus (HCV); or human immunodeficiency virus (HIV).
59. Tire method, or the combination, pharmaceutical composition, RNAi component, compound or interferon for use of any one of embodiments 1-58, wherein the subject is a virologically suppressed CHB-infected subject.

### EXAMPLES

The following examples are offered to illustrate but not to limit the invention. One of skill in the art will recognize that the following procedures may be modified using methods known to one of ordinary skill in the art.

### Example 1. Clinical Study of the Combination Therapy

A Phase 2, randomized, open-label, multicenter study is conducted to evaluate efficacy, pharmacokinetics, safety, and tolerability of response-guided treatment with combination regimens, including an RNAi component, a capsid assembly modulator and a nucleos(t)ide analog, with or without pegylated interferon alpha-2a, in treatment-naïve patients with HBeAg positive chronic hepatitis B virus infection and normal ALT. The study subject population includes adult males and females, aged 18-45 years (inclusive) of age or 18-55 years (inclusive) of age as per protocol amendment, with CHB who are HBeAg positive, are treatment-naïve, have HBV DNA≥20,000 IU/mL and have normal Alanine transaminase (ALT). It is targeted to enroll at least 30% participants with HBV DNA≥10⁷ IU/mL at screening in the study. The combination comprises Compound A, an RNAi component comprising an effective amount of a first RNAi agent comprising SEQ ID NO: 2 and SEQ ID NO: 11 and an effective amount of a second RNAi agent comprising SEQ ID NO: 16 and SEQ ID NO: 8 in a ratio of 2:1, an effective amount of tenofovir disoproxil, and optionally a PegIFN-α2a.

The study is conducted in 4 phases:
A screening phase (4-6 weeks)
An induction phase (Response-Guided Therapy (RGT) of 36-60 weeks (inclusive)).
A consolidation phase (12 weeks).
A follow-up (FU) phase (48 weeks).

The total duration of individual participation is between 100 and 126 weeks.

Enrolled participants will enter an induction phase with triple treatment of (1) 200 mg of the RNAi component as a subcutaneous injection in a single dose once every four weeks (Q4W), (2) 250 mg of compound A as oral tablets (25 mg and 100 mg) daily and (3) 245 mg of Tenofovir disoproxil (film-coated tablets) administered, as either tenofovir disoproxil fumarate or tenofovir disoproxil maleate, orally daily for a response-guided treatment (RGT) duration of ≥36 weeks to ≤60 weeks. End of the induction phase is defined by either meeting the study defined RGT criterion (e.g., HBsAg<10 IU/mL, HBeAg-negative, ALT<3×ULN, and/or HBV DNA<LLOQ) or reaching study Week 36 to 60, whichever comes first. The RGT criterion will be assessed from Week 36 onwards at each study visit, and the assessment will always be based on lab results from the previous study visit. If the RGT criterion is met, the participant will complete the induction phase at that visit.

While not wishing to be bound by theories, it is believed that the addition of short-term PegIFN-α2a to the regimen at a time when HBsAg is already significantly reduced or eliminated is expected to lead to reactivation of NK-cells. In addition, the reactivation of the innate immune system by PegIFN-α2a might lead to further activation of endogenous HBV-specific T-cells.

Upon completion of the induction phase, up to 80 participants will be randomized in a 1:1 ratio to one of the following intervention arms in the 12-week consolidation phase:
Arm 1: 200 mg RNAi component (injection Q4W);
   250 mg Compound A (tablets qd);
   245 mg tenofovir disoproxil (tablets qd); and
   180 μg PegIFN-α2a (injection, QW);
Arm 2: 200 mg RNAi (injection Q4W);
   250 mg Compound A (tablets qd); and
   245 tenofovir disoproxil (tablets qd).

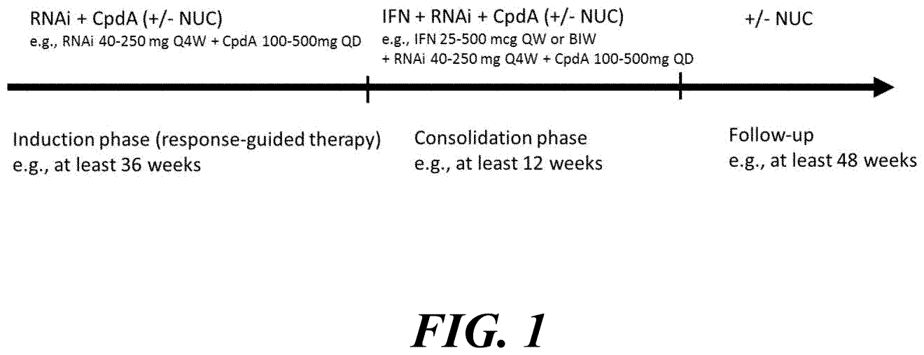

At the end of the consolidation phase, all participants will enter the FU phase and stop treatment with the RNAi component and compound A (and PegIFN-α2a for Arm 1). If NA treatment completion criteria (HBsAg<10 IU/mL, and HBeAg-negative, and HBV DNA <LLOQ, and ALT<3×upper limit of normal [ULN]) have been met at consolidation Week 12, NA will also be stopped at the next scheduled visit (i.e., FU Week 2), otherwise NA treatment will continue during the complete FU phase. Participants will be monitored closely during the 48-week FU phase and should restart NA treatment in accordance with the NA re-treatment criteria. The treatment completion criteria which take ALT, HBV DNA, HBeAg, and HBsAg levels into consideration, have been selected to ensure that only participants with a chance of sustained off-treatment response are allowed to complete all study intervention. Across a range of studies, HBsAg levels below 100 IU/mL are consistently associated with favorable off-treatment response. FIG. 1 shows a schematic overview of the study, wherein “RNAi” refers to the RNAi component, “CpdA” refers to compound A, “NUC” refers to a nucleotide (or nucleoside) analog, “IFN” refers to interferon, “Q4W” refers to once every 4 weeks, which encompasses the meaning of QM (once every month), “QW” means once every week, “QD” means once per day, “BIW” means twice weekly.

Safety and tolerability, including AEs, laboratory assessments, ECGs, vital signs, and physical examination, will be assessed throughout the study from the time that the ISA ICF is signed until the completion of the last study-related activity. Safety assessments included, e.g., vital sign assessment (resting heart rate, blood pressure, respiratory rate, temperature); clinical laboratory measurements (biochemistry, hematology, coagulation, urinalysis); resting ECG measurements; assessment of changes in patients' other concurrent medication/therapy; assessment of injection sites; and a 90-day post-EOS pregnancy follow-up call.

Efficacy will be evaluated using different parameters including, e.g., HBsAg, HBeAg, HBV DNA, and ALT, etc. Efficacy evaluation is made based on a combination of safety/tolerability data (occurrence of adverse events, blood sample analysis); pharmacokinetic data (RNAi component/NUC (nucleos(t)ide inhibitors)/compound A plasma concentrations); and pharmacodynamic data (immunology and virology assessments). The primary efficacy analysis will be performed when all participants have completed FU Week 24 or discontinued earlier. The primary efficacy endpoint, the proportion of participants with HBsAg seroclearance 24 weeks after stopping all study interventions of the consolidation phase and without restarting NA treatment, will be evaluated in both intervention arms. Secondary and exploratory efficacy endpoints will also be analyzed.

The study parameters are summarized in Table 4.

Table 4

TABLE 4

| | |
|---|---|
| Objectives | Endpoints |

Primary

| | |
|---|---|
| To evaluate the efficacy of a treatment | Proportion of participants with HBsAg |
| regimen of RANi component + | seroclearance 24 weeks after stopping |
| Compound A + NA with or without | all study interventions of the |
| PegIFN-α2a. | consolidation phase and without |
| | restarting NA treatment. |

Secondary

| | |
|---|---|
| To evaluate the safety and tolerability | Safety and tolerability including but not |
| of the study intervention. | limited to the proportion of participants |
| | with (serious) adverse events (S)AEs |
| | and abnormalities in clinical laboratory |
| | tests (including hematology, blood |
| | biochemistry, blood coagulation, |
| | urinalysis, urine chemistry, and renal |
| | biomarkers), 12-lead |
| | electrocardiograms (ECGs), vital signs, |
| | and physical examinations throughout |
| | the study. |
| To evaluate the efficacy of the study | Proportion of participants meeting RGT |
| intervention during the treatment period. | criterion (HBsAg <10 IU/mL) at or |
| | before Week 60 of the induction phase. |
| | Time to meet RGT criterion |
| | (HBsAg <10 IU/mL). |
| | Proportion of participants meeting the |
| | NA treatment completion criteria at the |
| | end of the consolidation phase. |
| To evaluate the efficacy of the study | Proportion of participants with HBsAg |
| intervention during the follow-up (FU) | seroclearance 48 weeks after stopping |
| phase. | all study interventions of the |
| | consolidation phase and without |
| | restarting NA treatment. |
| | Proportion of participants with HBV |
| | DNA <LLOQ 48 weeks after stopping |
| | all study interventions of the |
| | consolidation phase and without |
| | restarting NA treatment. |
| | Frequency of viral and/or biochemical |
| | flares and/or clinical flares. |
| | Proportion of participants requiring NA |
| | re-treatment. |
| To evaluate efficacy of the study | Proportion of participants with |
| intervention as measured by blood | (sustained) reduction, suppression, |
| markers (such as HBsAg, HBeAg, HBV | and/or seroclearance considering single |
| DNA, and alanine aminotransferase | and multiple markers (such as HBsAg, |
| [ALT]) during study intervention and | HBeAg, HBV DNA, and ALT). |
| follow-up. | Proportion of participants with HBsAg |
| | and HBeAg seroconversion. |
| | Change from baseline over time in |
| | HBsAg, HBeAg, and HBV DNA. |
| | Time to achieve HBsAg seroclearance, |
| | HBeAg seroclearance, and/or HBV |
| | DNA <LLOQ. |
| | Proportion of participants with HBeAg, |
| | HBsAg, and HBV DNA levels and/or |
| | changes from baseline below/above |
| | different cut-offs. |
| To evaluate the frequency of virologic | Proportion of participants with virologic |
| breakthrough. | breakthrough. |
| To evaluate the efficacy of NA re- | Proportion of participants who reach |
| treatment in participants who meet | HBV DNA undetectability after re-start |
| the criteria for NA re-treatment. | of NA treatment during follow-up. |
| To evaluate the pharmacokinetics (PK) | PK parameters of RANi component and |
| of the RANi component, Compound A, | Compound A |
| and optionally of NA and PegIFN-α2a. | Optionally, PK parameters of NA and/or |
| | PegIFN-α2a compared to historical data. |

Exploratory

| | |
|---|---|
| To identify baseline and on-treatment | Association of baseline characteristics |
| markers associated with efficacy. | and baseline/on-treatment viral blood |
| | markers (such as age, and baseline/ |
| | on-treatment HBsAg levels) with selected |
| | efficacy variables. |
| To explore changes in the severity of | Changes in fibrosis (according to |
| liver disease. | Fibroscan liver stiffness measurements) |
| | at end-of-study intervention (EOSI) and |
| | end of follow-up versus baseline. |
| To explore efficacy of the study | Changes from baseline in HBV RNA |
| intervention in terms of changes in HBV | and HBcrAg levels during the study |
| RNA and HBcrAg levels. | intervention and follow-up. |
| To explore the impact of study | Changes over time in score on the Short |
| intervention on participants' physical | Form 36 version 2 (SF-36v2). |
| and emotional functioning, and health- | Changes over time in score on the |
| related quality of life using patient- | Hepatitis B Quality of Life (HBQOL) |
| reported outcomes (PROs) during study | Instrument. |
| intervention and follow-up. | Changes over time in the 5-Level |
| | EuroQol 5-Dimension (EQ-5D-5L) |
| | Visual Analog Scale (VAS) score and |
| | Index score. |
| | Changes over time on the Patient Global |
| | Impression of Change (PGIC). |
| To explore the relationship between | Relationship between various plasma |
| plasma PK parameters (the RANi | PK parameters (the RANi component, |
| component, Compound A, NA, and/or | Compound A, NA, and/or pegIFN-a2a) |
| pegIFN-a2a) and selected | and selected efficacy and/or safety |
| pharmacodynamic (PD) parameters of | endpoints, as applicable. |
| efficacy and/or safety, as applicable. | |
| To explore the effect of PegIFN-α2a | Effect of PegIFN-α2a coadministration |
| coadministration on the PK of the RANi | on the PK of the RANi component and |
| component and Compound A (optional | Compound A. |
| PK substudy). | |
| To explore the HBV genome sequence | Assessment of intervention-associated |
| during study intervention and follow-up. | mutations. |
| To explore HBV-specific T-cell | Changes from baseline in HBV-specific |
| responses during study intervention and | peripheral blood T-cell responses. |
| follow-up.* | |

*Peripheral blood mononuclear cell (PBMC) samples for immune analyses will be collected at selected sites only.

### Example 2. Clinical Study of the Combination Therapy

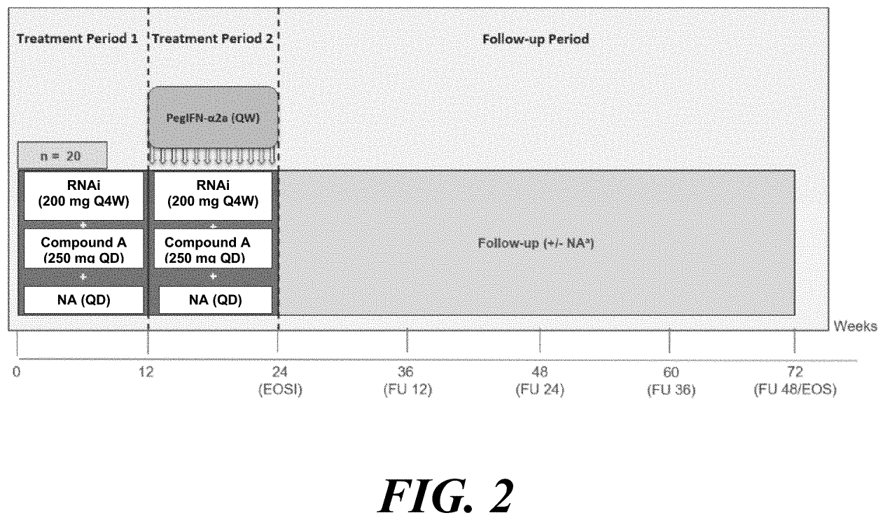

A Phase 2, randomized, open-label, single-arm, multicenter study is conducted to evaluate efficacy, safety, tolerability, and pharmacokinetics of treatment with combination regimens, including an RNAi component, a capsid assembly modulator, a nucleos(t)ide analog (NA), and pegylated interferon alpha-2a, in virologically suppressed patients with chronical hepatitis B (CHB) virus infection. A schematic overview of the study is shown in FIG. 2. The study subject population includes twenty (20) virologically suppressed CHB-infected participants, 18-65 years (inclusive) of age. It is targeted to enroll approximately 40% HBeAg-positive participants. The combination comprises an RNAi component comprising an effective amount of a first RNAi agent comprising SEQ ID NO: 2 and SEQ ID NO: 11 and an effective amount of a second RNAi agent comprising SEQ ID NO: 16 and SEQ ID NO: 8 in a ratio of 2:1; Compound A; a NA selected from tenofovir disoproxil (fumarate or maleate), tenofovir alafenamide (TAF), or entecavir (ETV); and a PegIFN-α2a.

Participants' CHB infection is documented by serum HBsAg positivity at screening. In addition, chronicity must be documented by any of the following, at least 6 months prior to screening: serum HBsAg positivity, HBeAg positivity or HBV DNA positivity, ALT elevation above ULN without another cause than HBV infection, documented transmission event. If none of the above are available, the following ways of documenting chronicity are acceptable at the time of screening: liver biopsy with changes consistent with chronic HBV, or absence of marker for acute HBV infection such as positive immunoglobulin M (IgM) anti-hepatitis B surface (HBs) and anti-HBc antibodies.

Participants should:
be on stable HBV treatment, defined as currently receiving NA treatment for at least 6 months prior to screening and having been on the same NA treatment regimen (at the same dose) as used in this study for at least 3 months at the time of screening, AND
have serum HBV DNA<60 IU/mL on 2 sequential measurements at least 6 months apart (one of which is at screening), AND
have documented ALT values <2. Ox ULN on 2 sequential measurements at least 6 months apart (one of which is at screening).

The study is conducted in 4 periods:
Screening period (4 weeks [if necessary, can be extended to a maximum of 8 weeks decided on a case-by-case basis and in agreement with the sponsor]).
Treatment Period 1 (12 weeks) consisting of combination treatment with RNAi component, Compound A, and NA.
Treatment Period 2 (12 weeks) consisting of combination treatment with RNAi component, Compound A, NA, and pegIFN-α2a.
Follow-up (FU) Period (48 weeks).

The total duration of individual participation is up to 76 weeks (including 4 weeks of screening).

Enrolled participants (n=20) will start Treatment Period 1 with the following combination treatment regimen for a duration of 12 weeks:
200 mg RNAi component (injection Q4W);
250 mg Compound A (tablets qd); and
NA (tablets QD): tenofovir disoproxil (245 mg), or TAF (25 mg), or ETV (0.5 mg).

At Week 12, participants who still meet the eligibility criteria for PegIFN-α2a will start Treatment Period 2 for a duration of 12 weeks:
200 mg RNAi component (injection Q4W);
250 mg Compound A (tablets qd);
NA (tablets QD): tenofovir disoproxil (245 mg), or TAF (25 mg), or ETV (0.5 mg); and
180 μg PegIFN-α2a (subcutaneous injection QW).

Key exclusion criteria for PegIFN-α2a include:
1. Patients with signs or symptoms compatible with autoimmune disorders.
2. Participants with bone marrow suppression.
3. Patients with hypoglycaemia, hyperglycaemia, and/or diabetes mellitus, who cannot be effectively controlled by medication.
4. Participants with pre-existing ophthalmologic disorders.
5. Participants with one or more of the following laboratory abnormalities:
   Absolute neutrophil count <1,500 cells/mm³.
   Serum creatinine >1.5×ULN.
   Inadequately controlled thyroid function (thyroid stimulating hormone [TSH] and thyroxine [T4]).
   CD4+ cell count <200 cells/mm³.
6. Participants with depression or other psychiatric disorders that are not adequately controlled on a stable medication regimen.

Participants no longer meeting the PegIFN-α2a eligibility criteria at Week 12 will continue with the Period 1 treatment until Week 24.

At Week 24, all participants stop treatment with RNAi component, Compound A, and PegIFN-α2a and start the FU Period. If the protocol-defined NA treatment completion criteria (HBsAg<10 IU/mL, and HBeAg-negative, and HBV DNA<LLOQ, and ALT<3× upper limit of normal [ULN]) have been met at Week 24, NA will be stopped at the next scheduled visit (i.e., FU Week 2), otherwise NA treatment continues during the complete FU Period. Participants who meet the protocol-defined NA treatment completion criteria are monitored closely during the 48-week FU Period and should re-start NA treatment immediately in the event of:
Signs of decreasing liver function based on laboratory findings (e.g., International Normalized Ratio [INR], direct bilirubin) or clinical assessment (e.g., ascites, hepatic encephalopathy).

In addition, re-start NA treatment should be considered in the following cases:
Confirmed HBeAg seroreversion (HBeAg positive after it was negative at NA completion), OR
Confirmed post-treatment increases in HBV DNA>2,000 IU/mL and ALT>5×ULN, OR
Confirmed post-treatment increases in HBV DNA>20,000 IU/mL.
Note: It is recommended that the initial HBV DNA and/or ALT test and the corresponding confirmation test used to decide on re-treatment are at least 4 weeks apart.
Note: The decision to re-start NA treatment should take into consideration the dynamics of HBV DNA and/or ALT values and should be discussed with the sponsor.

All participants will have sparse PK sampling during the treatment periods. Participants who consent to participate in the intensive PK substudy (optional) will also undergo intensive PK sampling.

Participants will be considered to have completed the study if they have completed all the assessments of the end of study (EOS) visit (i.e., FU Week 48).

An internal data review committee (DRC) is commissioned for monitoring safety of participants enrolled in this study. In addition, an Independent Flare Expert Panel (IFLEP) is appointed.

Safety and tolerability, including AEs, laboratory assessments, ECGs, vital signs, and physical examination, will be assessed throughout the study from the time that the ICF is signed until the completion of the last study-related activity. Safety assessments included, e.g., vital sign assessment (resting heart rate, blood pressure, respiratory rate, temperature); clinical laboratory measurements (biochemistry, hematology, coagulation, urinalysis); resting ECG measurements; assessment of changes in patients' other concurrent medication/therapy; assessment of injection sites; and a 90-day post-EOS pregnancy follow-up call.

Efficacy will be evaluated using different parameters including, e.g., HBsAg, HBeAg, HBV DNA, and ALT, etc. Efficacy evaluation is made based on a combination of safety/tolerability data (occurrence of adverse events, blood sample analysis); pharmacokinetic data (RNAi component/NUC (nucleos(t)ide inhibitors)/compound A plasma concentrations); and pharmacodynamic data (immunology and virology assessments). The primary efficacy analysis (with formal database lock) will be performed when all participants have completed Week 24 (EOSI) or discontinued earlier. The final analysis will be performed when all participants have completed the last study visit (FU Week 48) or discontinued earlier

The primary efficacy analysis will be performed when all participants have completed FU Week 24 or discontinued earlier. The primary endpoint is the proportion of participants with a reduction of at least 2 log₁₀ IU/mL in HBsAg levels from baseline to Week 24. Participants who do not have HBsAg data in the analysis window of Week 24 will be defined as none-responders. Secondary and exploratory efficacy endpoints will also be analyzed. Descriptive statistics and 90% confidence intervals (CIs) will be used to summarize all efficacy endpoints.

The study parameters are summarized in Table 5.

TABLE 5

| | |
|---|---|
| Objectives | Endpoints |

Primary

| | |
|---|---|
| To evaluate the efficacy in terms of | Proportion of participants with a |
| HBsAg levels of the study intervention | reduction of at least 2 log₁₀ IU/mL in |
| (i.e., a treatment regimen of RNAi | HBsAg levels from baseline to Week 24 |
| component + Compound A + NA + | (end of study intervention [EOSI]). |
| PegIFN-α2a). | |

Secondary

| | |
|---|---|
| To evaluate the safety and tolerability | Safety and tolerability including but not |
| of the study intervention. | limited to the proportion of participants |
| | with (S)AEs and abnormalities in clinical |
| | laboratory tests (including hematology, |
| | blood biochemistry, blood coagulation, |
| | urinalysis, urine chemistry, renal |
| | biomarkers), 12-lead ECGs, vital signs, |
| | and ophthalmic and physical |
| | examinations throughout the study. |
| To evaluate the efficacy of the study | Proportion of participants meeting the |
| intervention at the end of the 24-week | protocol-defined NA treatment |
| treatment period. | completion criteria at EOSI. |
| To evaluate the efficacy of the study | Proportion of participants with HBeAga, |
| intervention as measured by blood | HBsAg, HBV DNA, and ALT levels |
| markers (such as HBsAg, HBeAg^{a}, | below/above different cut-offs. |
| HBV DNA, and ALT) during the study | Proportion of participants with HBsAg |
| intervention and follow-up (FU) period. | and/or HBeAg^{a} seroconversion. |
| | Change from baseline over time in |
| | HBsAg, HBeAg^{a}, and/or HBV DNA. |
| | Time to achieve HBsAg and/or HBeAg^{a} |
| | seroclearance/seroconversion, and/or |
| | HBV DNA <LLOQ. |
| To evaluate the frequency of virologic | Proportion of participants with virologic |
| breakthrough^{b} during the 24-week | breakthrough^{b}. |
| treatment period, as well as during the | |
| FU period for participants who | |
| continue treatment with NA. | |
| To evaluate the efficacy of the study | Proportion of participants with HBsAg |
| intervention during the FU period. | seroclearance at Week 48 (i.e., 24 weeks |
| | after completion of all study |
| | interventions at Week 24) without re- |
| | starting NA treatment. |
| | Proportion of participants with HBV |
| | DNA <LLOQ at Week 48 (i.e., 24 weeks |
| | after completion of all study |
| | interventions at Week 24) without re- |
| | starting NA treatment. |
| | Frequency of virologic and/or |
| | biochemical flares. |
| | Proportion of participants requiring |
| | NA re-treatment. |
| To evaluate the PK of RNAi | PK parameters of RNAi component and |
| component, Compound A, and | Compound A. |
| optionally of NA, and PegIFN-α2a. | Optionally, PK parameters of NA and/or |
| | PegIFN-α2a compared to historical data. |

Exploratory

| | |
|---|---|
| To explore host and viral baseline and | Association of baseline characteristics |
| on-treatment markers associated with | and baseline/on-treatment host and viral |
| end of treatment and/or off-treatment | blood markers (such as age and HBsAg |
| response. | levels) with selected on or off-treatment |
| | efficacy variables. |
| To explore changes in the severity of | Changes in fibrosis (according to |
| liver disease. | Fibroscan liver stiffness measurements) |
| | at EOSI and the end of the FU period |
| | versus baseline. |
| To explore efficacy of the study | Changes from baseline in HBV RNA and |
| intervention in terms of changes in | HBcrAg levels over time. |
| HBV RNA and HBcrAg levels during | |
| the study intervention and FU period. | |
| To explore the relationship of PK with | Relationship of various PK parameters |
| selected pharmacodynamic (PD) | with selected efficacy and safety |
| parameters of efficacy and safety. | endpoints. |
| To explore the effect of PegIFN-α2a | Effect of PegIFN-α2a coadministration |
| coadministration on the PK of RNAi | on the PK of RNAi component and |
| component and Compound A (PK | Compound A. |
| substudy). | |
| To explore the HBV genome sequence | Assessment of intervention-associated |
| during the study intervention and FU | mutations over time. |
| period. | |
| To explore HBV-specific T-cell | Changes from baseline in HBV-specific |
| responses during the study intervention | peripheral blood T-cell responses over |
| and FU period.^{c} | time.^{c} |
| To explore the efficacy of NA re- | Proportion of participants who reach |
| treatment in participants who meet | HBV DNA <LLOQ after re-start of NA |
| the criteria for NA re-treatment. | treatment during the FU period. |

^{a}In HBeAg-positive participants only.
^{b}Virologic breakthrough: Confirmed on-treatment HBV DNA increase by >1 log₁₀ IU/mL from nadir or confirmed on-treatment HBV DNA level >200 IU/mL in participants who had HBV DNA level <LLOQ of the HBV DNA assay.
^{c}Peripheral blood mononuclear cell (PBMC) samples for immune analyses will be collected at selected sites only.

1. A method of treating a Hepatitis B virus (HBV) infection or a disease or disorder associated with the HBV infection in a subject in need thereof, comprising:
(a) administering to the subject a first pharmaceutical composition comprising an RNAi component having:
(i) a first RNAi agent comprising: an antisense strand comprising a nucleotide sequence of any one of the following: SEQ ID NON, SEQ ID NO:2, SEQ ID NO:3, SEQ ID NO:4, SEQ ID NO:5, SEQ ID NO:6, and SEQ ID NO:7 and a sense strand comprising a nucleotide sequence selected from the group consisting of: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NO:8 and SEQ ID NO:9, and a sense strand comprising a nucleotide sequence selected from the group consisting of: SEQ ID NO: 16, SEQ ID NO: 17, SEQ ID NO: 18, and SEQ ID NO: 19; and
(b) administering to the subject a second pharmaceutical composition comprising a capsid assembly modulator compound of Formula (I): 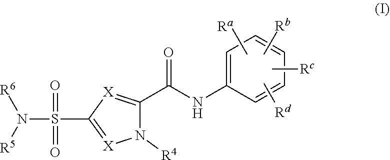
or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and
(c) administering to the subject a third pharmaceutical composition comprising an interferon.

2. (canceled)

3. (canceled)

4. (canceled)

5. The method of claim 1, wherein the first, second, and third pharmaceutical compositions are for simultaneous, separate or sequential administration.

6. The method of claim 1, wherein the interferon is selected from the group consisting of interferon alpha, interferon lambda, pegylated interferon, pegylated interferon alpha-2α, and pegylated interferon lambda-1α.

7. The method of claim 1, wherein the first RNAi agent or the second RNAi agent comprises at least one modified nucleotide or at least one modified internucleoside linkage, and substantially all of the nucleotides in the first and the second RNAi agents are modified nucleotides.

8. The method of claim 1, wherein the first RNAi agent or the second RNAi agent further comprises a targeting ligand which is conjugated to the first RNAi agent or the second RNAi agent, wherein the targeting ligand comprises N-acetyl-galactosamine and is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

9. The method of claim 8, wherein the targeting ligand is conjugated to the sense strand of the first RNAi agent or the second RNAi agent and the targeting ligand is conjugated to the 5′ terminus of the sense stand of the first or the second RNAi agent.

10. The method of claim 1, wherein the first RNAi agent and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO: 18.

11. The method of claim 1, wherein the first RNAi agent and the second RNAi agents are each independently conjugated to a targeting ligand comprising N-acetyl-galactosamine, and the first and the second RNAi agents independently comprise a duplex selected from the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

12. The method of claim 1, wherein the ratio of the first RNAi agent to the second RNAi agent by weight is in the range of about 1:2 to about 5:1, such as about 2:1.

13. The method of claim 1, wherein the compound of Formula (I) is a compound of Formula (II): 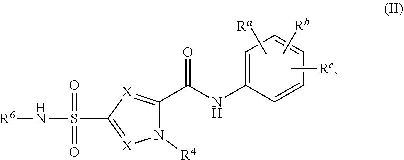
a metabolite or pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃, preferably,
R^{a}, R^{b} and R^{c} are independently hydrogen, fluoro, bromo, chloro, or CN,
R⁴ is C₁-C₃ alkyl, more preferably R⁴ is methyl,
R⁶ is C₂-C₆ alkyl optionally substituted with one or more of—OH, fluoro, or C₁-C₄ alkyl optionally substituted with R¹⁰, more preferably R⁶ is C₂-C₆ alkyl substituted with one or more fluoro, and
each R⁷ is independently hydrogen, halogen or methyl, more preferably at least one R⁷ is hydrogen.

14. The method of claim F wherein the compound of Formula (I) is selected from the group consisting of: 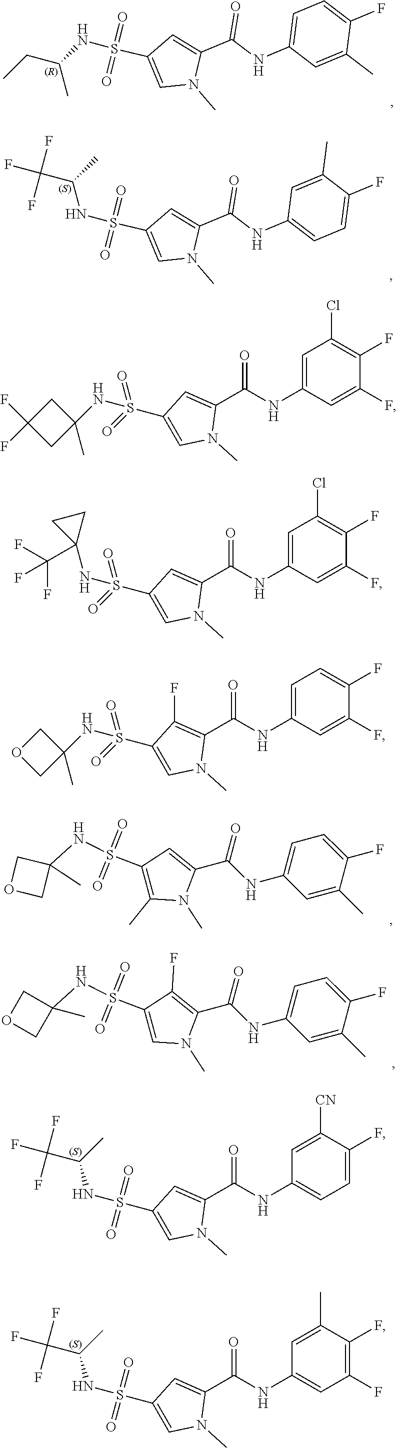 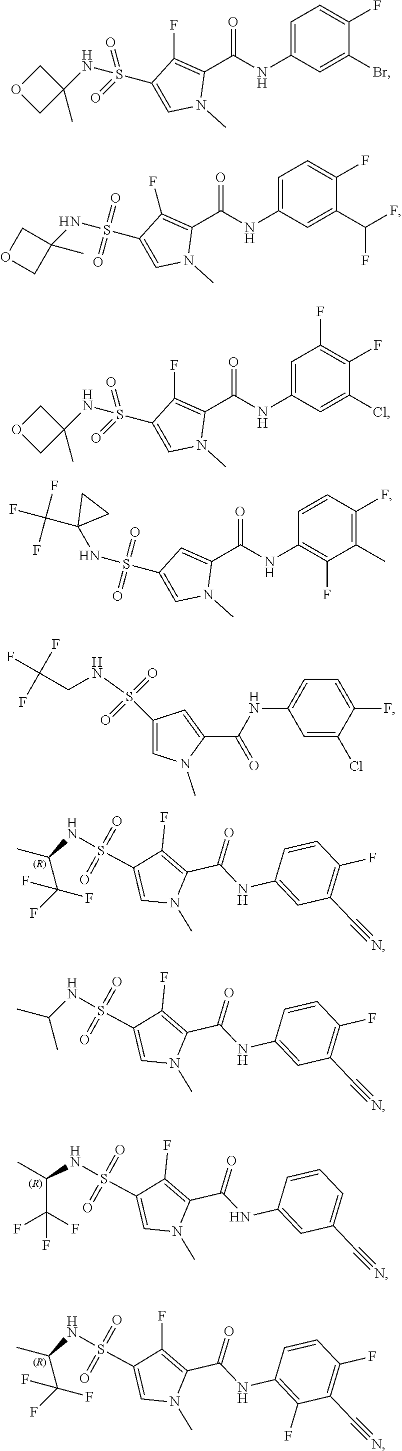 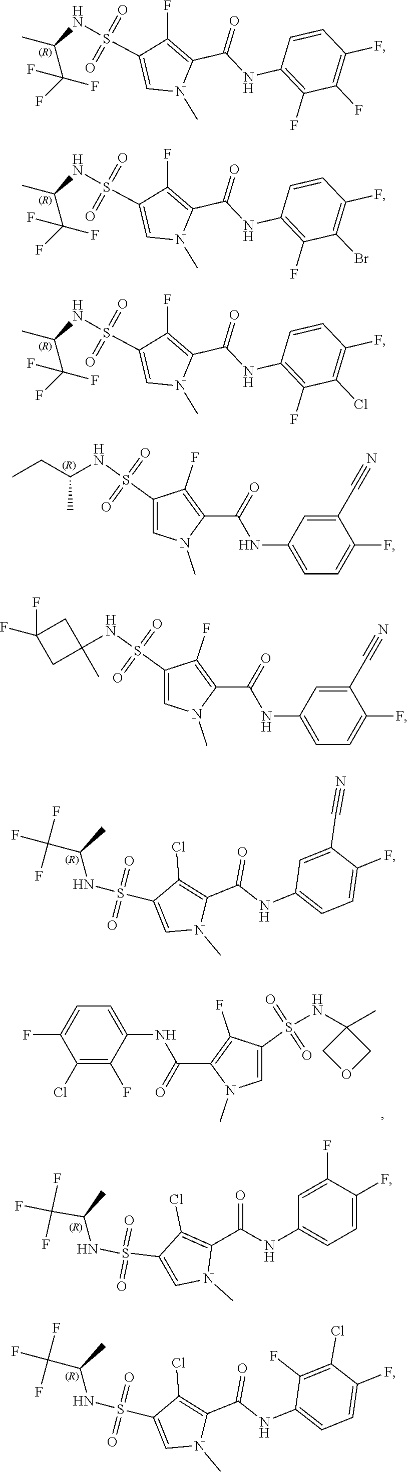 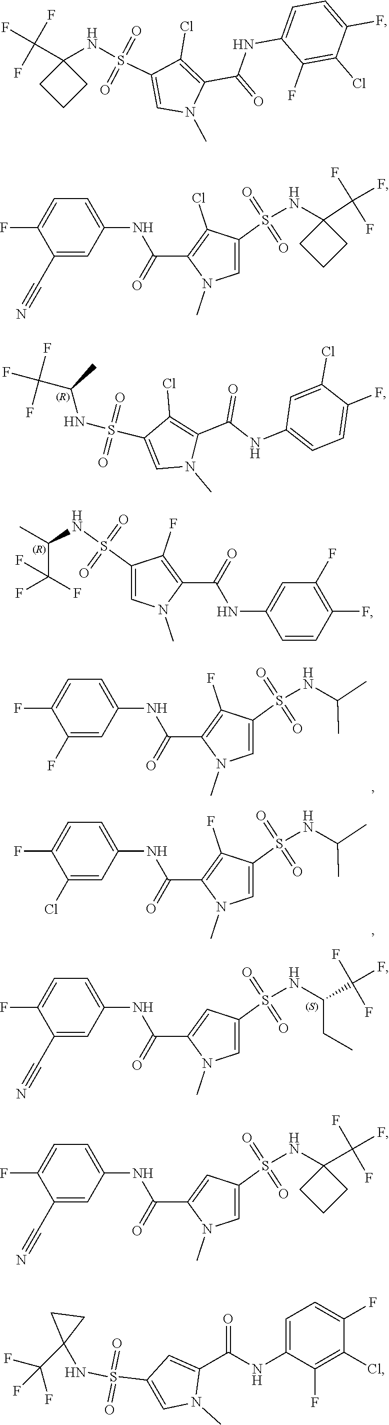 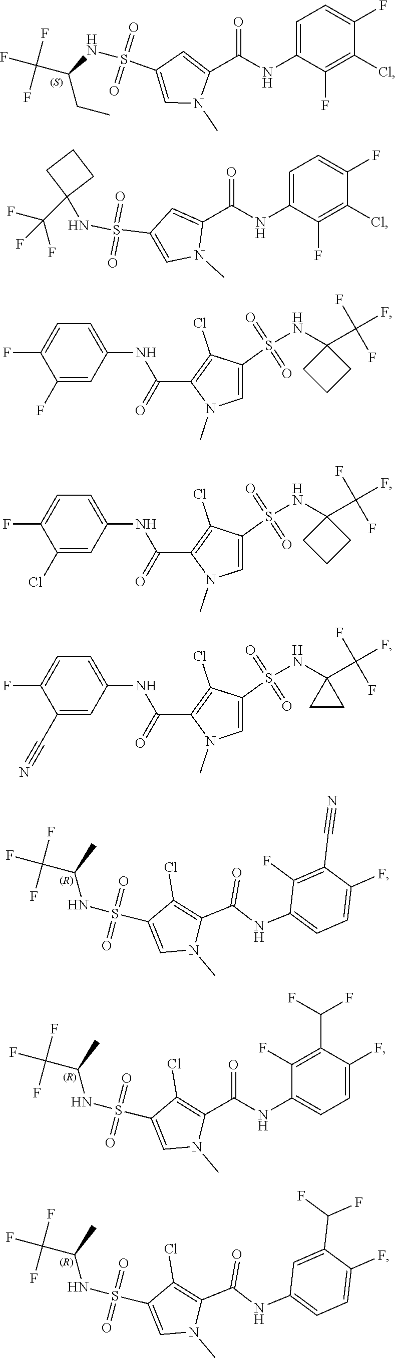 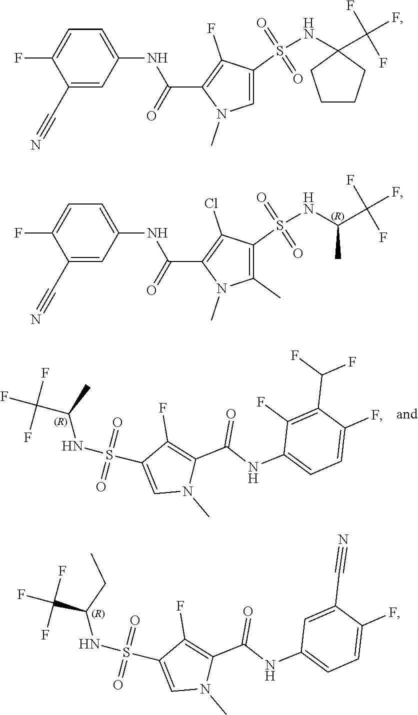
or a pharmaceutically acceptable salt thereof.

15. The method of claim 14, wherein the compound of Formula (I) is 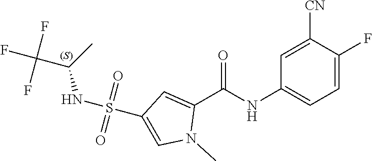
or a pharmaceutically acceptable salt thereof.

16. The method of claim 14, wherein the compound of Formula (I) is 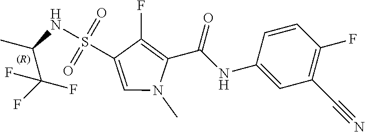
or a pharmaceutically acceptable salt thereof.

17. The method of claim 15,
wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) is 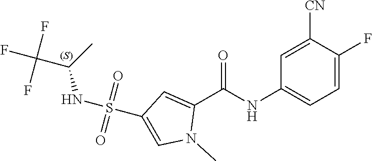
or a pharmaceutically acceptable salt thereof, and the interferon is pegylated interferon alpha-2a or pegylated interferon lambda-1a.

18. The method of claim 16,
wherein the first RNAi agent and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16, the compound of Formula (I) is 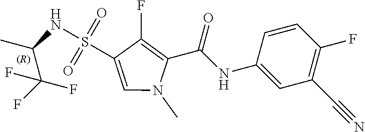
or a pharmaceutically acceptable salt thereof, and the interferon is pegylated interferon alpha-2α or pegylated interferon lambda-1α.

19. The method of claim 1, wherein the RNAi component is administered to the subject once a month at a dose of about 40-250 mg, 40-200 mg, or 200 mg, via intravenous or subcutaneous injection.

20. The method of claim 1, wherein the compound of Formula (I) or the pharmaceutically acceptable salt thereof is administered to the subject once a day at a dose of about 100-500 mg, 200-300 mg, or 250 mg. via oral administration.

21. The method of claim 1, wherein the interferon is administered to the subject in a once a week dose of about 25-500 meg, 80-300 meg, 100-200 mcg, or 180 mcg, via intravenous or subcutaneous injection.

22. The method of claim 1, wherein the pharmaceutical composition comprising the RNAi component is administered simultaneously or sequentially with the compound of Formula (I) or the pharmaceutically acceptable salt thereof.

23. The method of claim 1, wherein the pharmaceutical composition comprising the RNAi component is administered separately from the compound of Formula (I) or the pharmaceutically acceptable salt thereof.

24. The method of claim 1, wherein the compound of Formula (I) or the pharmaceutically acceptable salt thereof is administered to the human subject for at least about 1 month prior to the administration of the pharmaceutical composition comprising the RNAi component and/or the interferon.

25. The method of claim 1, wherein the human subject is further treated with a nucleoside analog or a nucleotide analog.

26. The method of claim 25, wherein the nucleotide analog is entecavir, which is administered to the subject once a day at a dose of about 0.1-5 mg.

27. The method of claim 25, wherein the nucleotide analog is tenofovir alafenamide, which is administered to the human subject once a day at a dose of about 5-50 mg or the nucleotide analog is tenofovir disoproxil fumarate, which is administered to the subject once a day at a dose of about 200-500 mg.

28. The method of claim 1, wherein the human subject has a chronic HBV infection and is immune tolerant.

29. The method of claim 1, wherein the administration of the first, second, and third pharmaceutical compositions ceases when the human subject meets at least one of, at least two of the following criteria:
i. a serum HBV DNA lower than the lower limit of quantification (LLoQ), wherein the lower limit of quantification is lower than 20 IU/mL, or is lower than 20 IU/mL, or is lower than 15 IU/mL,
ii. a serum ALT concentration lower than 3 times the upper normal limit, or lower than 129 U/L if the subject is a male human subject or lower than 108 U/L if the human subject is a female human subject, or a serum ALT concentration lower than 120 U/L if the human subject is a male human subject or lower than 105 U/L if the subject is a female human subject, or a serum ALT concentration lower than 90 U/L if the subject is a male subject or lower than 57 U/L if the subject is a female human subject,
iii. a HBeAg-negative serum, and
iv. a serum HBsAg of 100 IU/mL or lower, or 10 IU/mL or lower.

30. The method of claim 1, wherein the first, second, and third pharmaceutical compositions are administered to the subject for 10-80 weeks, for 12-72 weeks, for 12-60 weeks, for 12-48 weeks, or for 12 weeks.

31. The method of claim 29, wherein the human subject is further optionally treated with a nucleoside analog or a nucleotide analog selected from the group consisting of entecavir, tenofovir disoproxil fumarate, and tenofovir alafenamide, and wherein the administration of the nucleoside analog or the nucleotide analog is optionally continued after the administration of the first, second, and third pharmaceutical compositions ceases.

32. The method of claim 1, wherein, prior to administering the third pharmaceutical composition comprising the interferon, the subject is administered with the first pharmaceutical composition comprising the RNAi component and the second pharmaceutical composition comprising the compound of formula (I) or the pharmaceutically acceptable salt thereof until the HBsAg concentration of the serum of the subject is 100 IU/mL or lower and/or for a duration of 36-60 weeks.

33. The method of claim 32, wherein the administrations of the first pharmaceutical composition and the second pharmaceutical compositions continue after the administration of the third pharmaceutical composition, wherein the administration of the first pharmaceutical composition, the second pharmaceutical composition and third pharmaceutical compositions continue for 12 weeks after the initial administration of the third pharmaceutical composition.

34. The method of claim 32, wherein the human subject is further treated with a nucleoside analog or a nucleotide analog selected from the group consisting of entecavir, tenofovir disoproxil fumarate, and tenofovir alafenamide.

35. The method of claim 1, wherein the RNAi component is administered by intravenous or subcutaneous injection-once a month at a dose of about 40-250 mg, 40-200 mg, or 200 mg; the compound of Formula (I) or a pharmaceutically acceptable salt thereof is administered orally once a day at a dose of about 100-500 mg, 200-300 mg, or 250 mg; and the interferon is administered once per week at a dose of about 25-500 mcg per week, 80-300 mcg per week, 100-200 mcg per week, or 180 mcg per week via intravenous or subcutaneous injection.

36. A pharmaceutical combination for treating Hepatitis B virus (HBV) infection or a disease or disorder associated with the HBV infection in a subject in need thereof, comprising:
(a) a first pharmaceutical composition comprising an RNAi component having:
(i) a first RNAi agent comprising: an antisense strand comprising a nucleotide sequence of any one of the following: SEQ ID NON, SEQ ID NO:2, SEQ ID NO:3, SEQ ID NO:4, SEQ ID NO:5, SEQ ID NO:6, and SEQ ID NO:7 and a sense strand comprising a nucleotide sequence selected from the group consisting of: SEQ ID NO: 10, SEQ ID NO: 11, SEQ ID NO: 12, SEQ ID NO: 13, SEQ ID NO: 14, and SEQ ID NO: 15; and
(ii) a second RNAi agent comprising: an antisense strand comprising a nucleotide sequence of any one of the following: SEQ ID NO:8 and SEQ ID NOV, and a sense strand comprising a nucleotide sequence selected from the group consisting of: SEQ ID NO:16, SEQ ID NO: 17, SEQ ID NO: 18, and SEQ ID NO: 19; and
(b) a second pharmaceutical composition comprising a capsid assembly modulator compound of Formula (I): 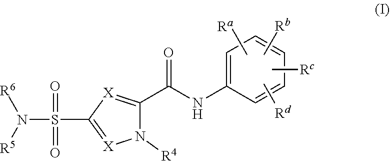
or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, -ocF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl; and
(c) a third pharmaceutical composition comprising an interferon.

37. The pharmaceutical combination of claim 36, wherein the first RNAi agent and the second RNAi agent are each independently conjugated to a targeting ligand comprising N-acetyl-galactosamine, and the first and the second RNAi agents independently comprise a duplex selected from the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

38. The pharmaceutical composition of claim 36, wherein the ratio of the first RNAi agent to the second RNAi agent by weight is in the range of about 1:2 to about 5:1, such as about 2:1.

39. The pharmaceutical composition of claim 36, wherein, wherein the compound of Formula (I) is a compound of Formula (II): 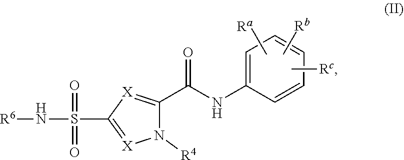
a metabolite or pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃, preferably,
R^{a}, R^{b} and R^{c} are independently hydrogen, fluoro, bromo, chloro, or CN,
R⁴ is C₁-C₃ alkyl, more preferably R⁴ is methyl,
R⁶ is C₂-C₆ alkyl optionally substituted with one or more of—OH, fluoro, or C₁-C₄ alkyl optionally substituted with R¹⁰, more preferably R⁶ is C₂-C₆ alkyl substituted with one or more fluoro, and
each R⁷ is independently hydrogen, halogen or methyl, more preferably at least one R⁷ is hydrogen.

40. The pharmaceutical composition of claim 36, wherein the compound of Formula (I) is selected from the group consisting of: 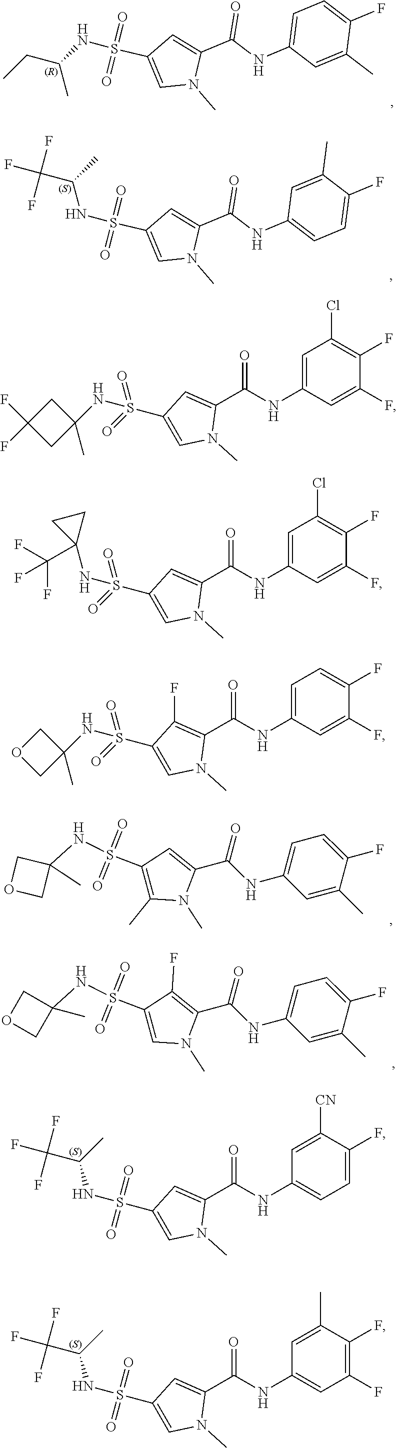 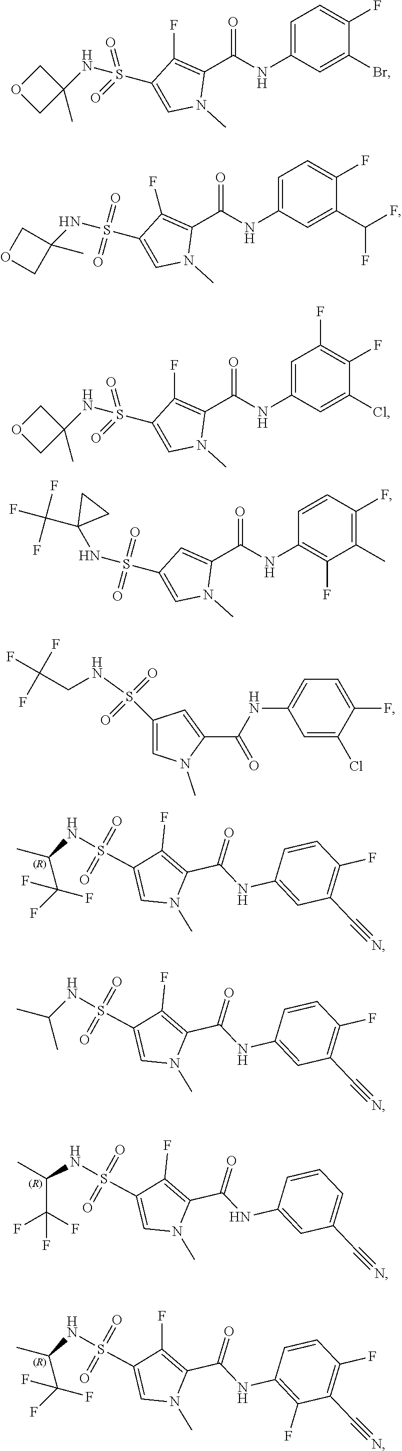 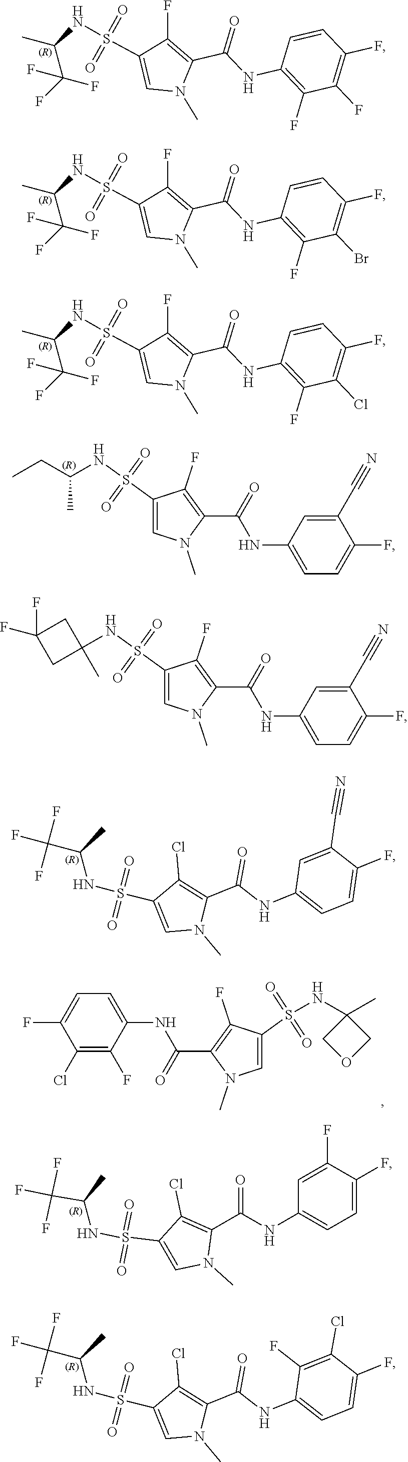 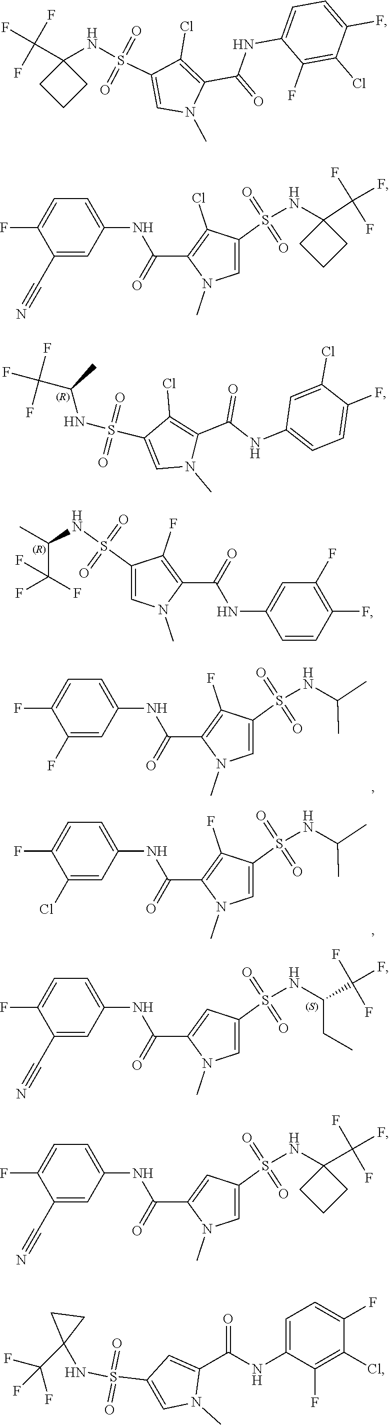 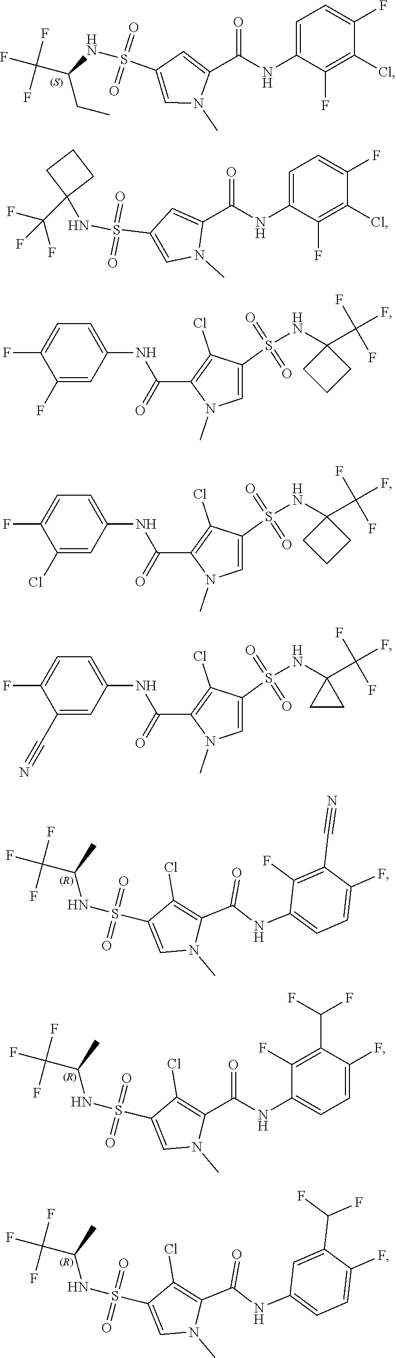 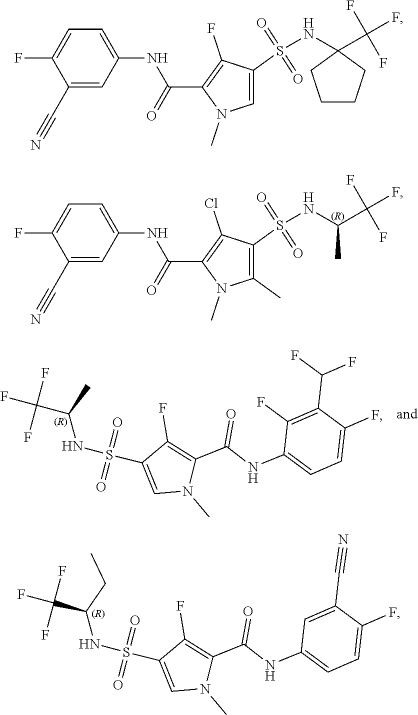
or a pharmaceutically acceptable salt thereof.

41. The pharmaceutical combination of claim 36, wherein the first and the second RNAi agents are each independently conjugated to (NAG37)s,
wherein the first RNAi agent comprises an antisense strand comprising SEQ ID NO: 2 and a sense strand comprising SEQ ID NO: 11,
wherein the second RNAi agent comprises an antisense strand comprising SEQ ID NO: 8 and a sense strand comprising SEQ ID NO: 16,
wherein the compound of Formula (I) is 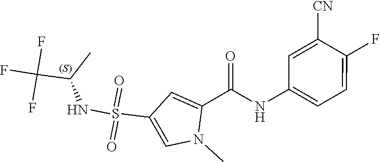
or a pharmaceutically acceptable salt thereof, and
wherein the interferon is pegylated interferon alpha-2a or pegylated interferon lambda-1a.

42. The pharmaceutical combination of claim 36, wherein the first and the second RNAi agents are each independently conjugated to (NAG37)s,
wherein the first RNAi agent comprises an antisense strand comprising SEQ ID NO: 2 and a sense strand comprising SEQ ID NO: 11,
wherein the second RNAi agent comprises an antisense strand comprising SEQ ID NO: 8 and a sense strand comprising SEQ ID NO: 16,
wherein the compound of Formula (I) is 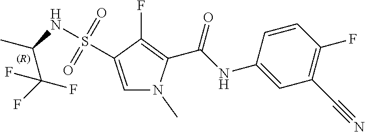
or a pharmaceutically acceptable salt thereof, Wherein and the interferon is pegylated interferon alpha-2α or pegylated interferon lambda-1α.


SEQUENCE LISTING (19 sequences)

SEQ ID NO: 1 - RNA, 21 nt, Artificial Sequence
  HBV RNAi Agent antisense strand modified sequence
agaaaauugagagaaguccac

SEQ ID NO: 2 - RNA, 21 nt, Artificial Sequence
  HBV RNAi Agent antisense strand modified sequence
agaaaauugagagaaguccac

SEQ ID NO: 3 - RNA, 23 nt, Artificial Sequence
  HBV RNAi Agent antisense strand modified sequence
agaaaauugagagaaguccacuu

SEQ ID NO: 4 - RNA, 22 nt, Artificial Sequence
  HBV RNAi Agent antisense strand modified sequence
agaaaauugagagaaguccacc

SEQ ID NO: 5 - RNA, 21 nt, Artificial Sequence
  HBV RNAi Agent antisense strand unmodified sequence
agaaaauugagagaaguccac

SEQ ID NO: 6 - RNA, 23 nt, Artificial Sequence
  HBV RNAi Agent antisense strand unmodified sequence
agaaaauugagagaaguccacuu

SEQ ID NO: 7 - RNA, 22 nt, Artificial Sequence
  HBV RNAi Agent antisense strand unmodified sequence
agaaaauugagagaaguccacc

SEQ ID NO: 8 - RNA, 21 nt, Artificial Sequence
  HBV RNAi Agent antisense strand modified sequence
uaccaauuuaugccuacagcg

SEQ ID NO: 9 - RNA, 21 nt, Artificial Sequence
  HBV RNAi Agent antisense strand unmodified sequence
uaccaauuuaugccuacagcg

SEQ ID NO: 10 - RNA, 21 nt, Artificial Sequence
  HBV RNAi Agent sense strand modified sequence
guggacuucucucaauuuucu

SEQ ID NO: 11 - RNA, 21 nt, Artificial Sequence
  HBV RNAi Agent sense strand modified sequence
guggacuucucucaauuuucu

SEQ ID NO: 12 - RNA, 22 nt, Artificial Sequence
  HBV RNAi Agent sense strand modified sequence
gguggacuucucucaauuuucu

SEQ ID NO: 13 - RNA, 21 nt, Artificial Sequence
  HBV RNAi Agent sense strand modified sequence
guggacuucucucaauuuucu

SEQ ID NO: 14 - RNA, 21 nt, Artificial Sequence
  HBV RNAi Agent sense strand unmodified sequence
guggacuucucucaauuuucu

SEQ ID NO: 15 - RNA, 22 nt, Artificial Sequence
  HBV RNAi Agent sense strand unmodified sequence
gguggacuucucucaauuuucu

SEQ ID NO: 16 - RNA, 21 nt, Artificial Sequence
  HBV RNAi Agent sense strand modified sequence
cgcuguaggcauaaauuggua

SEQ ID NO: 17 - RNA, 21 nt, Artificial Sequence
  HBV RNAi Agent sense strand modified sequence
cgcuguaggcauaaauuggua

SEQ ID NO: 18 - RNA, 21 nt, Artificial Sequence
  HBV RNAi Agent sense strand modified sequence
cgcuguaggcauaaauuggua

SEQ ID NO: 19 - RNA, 21 nt, Artificial Sequence
  HBV RNAi Agent sense strand unmodified sequence
cgcuguaggcauaaauuggua